SEQUENCE LISTING (20 sequences)

SEQ ID NO: 1 - DNA, 7310 bp, Senecavirus A
  misc_feature 1..7310: note = SVV-001
  source 1..7310: mol_type = genomic DNA
tttgaaatggggggctgggccctgatgcccagtccttcctttccccttccggggggttaaccggctgtgtttgctagaggcacagaggggcaacatccaacctgcttttgcggggaacggtgcggctccgattcctgcgtcgccaaaggtgttagcgcacccaaacggcgcacctaccaatgttattggtgtggtctgcgagttctagcctactcgtttctcccccgaccattcactcacccacgaaaagtgtgttgtaaccataagatttaacccccgcacgggatgtgcgataaccgtaagactggctcaagcgcggaaagcgctgtaaccacatgctgttagtccctttatggctgcaagatggctacccacctcggatcactgaactggagctcgaccctccttagtaagggaaccgagaggccttcgtgcaacaagctccgacacagagtccacgtgactgctaccaccatgagtacatggttctcccctctcgacccaggacttctttttgaatatccacggctcgatccagagggtggggcatgacccctagcatagcgagctacagcgggaactgtagctaggccttagcgtgccttggatactgcctgatagggcgacggcctagtcgtgtcggttctataggtagcacatacaaatatgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactgacccagatagcccaaggcgcttcggtgctgccggcgattctgggagaactcagtcggaacagaaaagggaaaaaaaaaaaaaaaaaaaaaaaaaaaaaa

SEQ ID NO: 2 - DNA, 6546 bp, Senecavirus A
  misc_feature 1..6546: note = SVV-001 nucleotides 667-7212, NC_011349.1
  source 1..6546: mol_type = genomic DNA
atgcagaactctcatttttctttcgatacagcctctggcacctttgaagatgtaaccggaacaaaagtcaagatcgttgaataccccagatcggtgaacaatggtgtttacgattcgtctactcatttggagatactgaacctacagggtgaaattgaaattttaaggtctttcaatgaataccaaattcgcgccgccaaacaacaactcggactggacatcgtgtacgaactacagggtaatgttcagacaacgtcaaagaatgattttgattcccgtggcaataatggtaacatgaccttcaattactacgcaaacacttatcagaattcagtagacttctcgacctcctcgtcggcgtcaggcgccggacccgggaactcccggggcggattagcgggtctcctcacaaatttcagtggaatcttgaaccctcttggctacctcaaagatcacaacaccgaagaaatggaaaactctgctgatcgagtcacaacgcaaacggcgggcaacactgccataaacacgcaatcatcattgggtgtgttgtgtgcctacgttgaagacccgaccaaatctgatcctccgtccagcagcacagatcaacccaccaccactttcactgccatcgacaggtggtacactggacgtctcaattcttggacaaaagctgtaaaaaccttctcttttcaggccgtcccgcttcccggggcctttctgtctaggcagggaggcctcaacggaggggccttcacagctaccctacatagacactttttgatgaagtgcgggtggcaggtgcaggtccaatgtaatttgacacaattccaccaaggcgctcttcttgttgccatggttcctgaaaccacccttgatgtcaagcccgacggtaaggcaaagagcttacaggagctgaatgaagaacagtgggtggaaatgtctgacgattaccggaccgggaaaaacatgccttttcagtctcttggcacatactatcggccccctaactggacttggggtcccaatttcatcaacccctatcaagtaacggttttcccacaccaaattctgaacgcgagaacctctacctcggtagacataaacgtcccatacatcggggagacccccacgcaatcctcagagacacagaactcctggaccctcctcgttatggtgctcgttcccctagactataaggaaggagccacaactgacccagaaattacattttctgtaaggcctacaagtccctacttcaatgggcttcgcaaccgctacacggccgggacggacgaagaacaggggcccattcctacggcacccagagaaaattcgcttatgtttctctcaaccctccctgacgacactgtccctgcttacgggaatgtgcgtacccctcctgtcaattacctccctggtgaaataaccgaccttttgcaactggcccgcatacccactctcatggcatttgagcgggtgcctgaacccgtgcctgcctcagacacatatgtgccctacgttgccgttcccacccagttcgatgacaggcctctcatctccttcccgatcaccctttcagatcccgtctatcagaacaccctggttggcgccatcagttcaaatttcgccaattaccgtgggtgtatccaaatcactctgacattttgtggacccatgatggcgagagggaaattcctgctctcgtattctcccccaaatggaacgcaaccacagactctttccgaagctatgcagtgcacatactctatttgggacataggcttgaactctagttggaccttcgtcgtcccctacatctcgcccagtgactaccgtgaaactcgagccattaccaactcggtttactccgctgatggttggtttagcctgcacaagttgaccaaaattactctaccacctgactgtccgcaaagtccctgcattctctttttcgcttctgctggtgaggattacactctccgtctccccgttgattgtaatccttcctatgtgttccactccaccgacaacgccgagaccggggttattgaggcgggtaacactgacaccgatttctctggtgaactggcggctcctggctctaaccacactaatgtcaagttcctgtttgatcgatctcgattattgaatgtaatcaaggtactggagaaggacgccgttttcccccgccctttccctacacaagaaggtgcgcagcaggatgatggttacttttgtcttctgaccccccgcccaacagtcgcttcccgacccgccactcgtttcggcctgtacgccaatccgtccggcagtggtgttcttgctaacacttcactggacttcaatttttatagcttggcctgtttcacttactttagatcggaccttgaggttacggtggtctcactagagccggatctggaatttgctgtagggtggtttccttctggcagtgaataccaggcttccagctttgtctacgaccagctgcatgtgcccttccactttactgggcgcactccccgcgctttcgctagcaagggtgggaaggtatctttcgtgctcccttggaactctgtctcgtctgtgctccccgtgcgctgggggggggcttccaagctctcttctgctacgcggggtctaccggcgcatgctgattgggggactatttacgcctttgtcccccgtcctaatgagaagaaaagcaccgctgtaaaacacgtggccgtgtacattcggtacaagaacgcacgtgcctggtgccccagcatgcttccctttcgcagctacaagcagaagatgctgatgcaatctggcgatatcgagaccaatcctggtcctgcttctgacaacccaattttggagtttcttgaagcagaaaatgatctagtcactctggcctctctctggaagatggtgcactctgttcaacagacctggagaaagtatgtgaagaacgatgatttttggcccaatttactcagcgagctagtgggggaaggctctgtcgccttggccgccacgctatccaaccaagcttcagtaaaggctcttttgggcctgcactttctctctcgggggctcaattacactgacttttactctttactgatagagaaatgctctagtttctttaccgtagaaccacctcctccaccagctgaaaacctgatgaccaagccctcagtgaagtcgaaattccgaaaactgtttaagatgcaaggacccatggacaaagtcaaagactggaaccaaatagctgccggcttgaagaattttcaatttgttcgtgacctagtcaaagaggtggtcgattggctgcaggcctggatcaacaaagagaaagccagccctgtcctccagtaccagttggagatgaagaagctcgggcctgtggccttggctcatgacgctttcatggctggttccgggccccctcttagcgacgaccagattgaatacctccagaacctcaaatctcttgccctaacactggggaagactaatttggcccaaagtctcaccactatgatcaatgccaaacaaagttcagcccaacgagttgaacccgttgtggtggtccttagaggcaagccgggatgcggcaagagcttggcctctacgttgattgcccaggctgtgtccaagcgcctctatggctcccaaagtgtatattctcttcccccagatccagatttcttcgatggatacaaaggacagttcgtgaccttgatggatgatttgggacaaaacccggatggacaagatttctccaccttttgtcagatggtgtcgaccgcccaatttctccccaacatggcggaccttgcagagaaagggcgtccctttacctccaatctcatcattgcaactacaaatctcccccacttcagtcctgtcaccattgctgatccttctgcagtctctcgccgtatcaactacgatctgactctagaagtatctgaggcctacaagaaacacacacggctgaattttgacttggctttcaggcgcacagacgccccccccatttatccttttgctgcccatgtgccctttgtggacgtagctgtgcgcttcaaaaatggtcaccagaattttaatctcctagagttggtcgattccatttgtacagacattcgagccaagcaacaaggtgcccgaaacatgcagactctggttctacagagccccaacgagaatgatgacacccccgtcgacgaggcgttgggtagagttctctcccccgctgcggtcgatgaggcgcttgtcgacctcactccagaggccgacccggttggccgtttggctattcttgccaagctaggtcttgccctagctgcggtcacccctggtctgataatcttggcagtgggactctacaggtacttctctggctctgatgcagaccaagaagaaacagaaagtgagggatctgtcaaggcacccaggagcgaaaatgcttatgacggcccgaagaaaaactctaagccccctggagcactctctctcatggaaatgcaacagcccaacgtggacatgggctttgaggctgcggtcgctaagaaagtggtcgtccccattaccttcatggttcccaacagaccttctgggcttacacagtccgctcttctggtgaccggccggaccttcctaatcaatgaacatacatggtccaatccctcctggaccagcttcacaatccgcggtgaggtacacactcgtgatgagcccttccaaacggttcatttcactcaccacggtattcccacagatctgatgatggtacgtctcggaccgggcaattctttccctaacaatctagacaagtttggacttgaccagatgccggcacgcaactcccgtgtggttggcgtttcgtccagttacggaaacttcttcttctctggaaatttcctcggatttgttgattccatcacctctgaacaaggaacttacgcaagactctttaggtacagggtgacgacctacaaaggatggtgcggctcggccctggtctgtgaggccggtggcgtccgacgcatcattggcctgcattctgctggcgccgccggtatcggcgccgggacctatatctcaaaattaggactaatcaaagccctgaaacacctcggtgaacctttggccacaatgcaaggactgatgactgaattagagcctggaatcaccgtacatgtaccccggaaatccaaattgagaaagacgaccgcacacgcggtgtacaaaccggagtttgagcctgctgtgttgtcaaaatttgatcccagactgaacaaggatgttgacttggatgaagtaatttggtctaaacacactgccaatgtcccttaccaacctcctttgttctacacatacatgtcagagtacgctcatcgagtcttctccttcttggggaaagacaatgacattctgaccgtcaaagaagcaattctgggcatccccggactagaccccatggatccccacacagctccgggtctgccttacgccatcaacggccttcgacgtactgatctcgtcgattttgtgaacggtacagtagatgcggcgctggctgtacaaatccagaaattcttagacggtgactactctgaccatgtcttccaaacttttctgaaagatgagatcagaccctcagagaaagtccgagcgggaaaaacccgcattgttgatgtgccctccctggcgcattgcattgtgggcagaatgttgcttgggcgctttgctgccaagtttcaatcccatcctggctttctcctcggctctgctatcgggtctgaccctgatgttttctggaccgtcataggggctcaactcgaggggagaaagaacacgtatgacgtggactacagtgcctttgactcttcacacggcactggctccttcgaggctctcatctctcactttttcaccgtggacaatggttttagccctgcgctgggaccgtatctcagatccctggctgtctcggtgcacgcttacggcgagcgtcgcatcaagattaccggtggcctcccctccggttgtgccgcgaccagcctgctgaacacagtgctcaacaatgtgatcatcaggactgctctggcattgacttacaaggaatttgaatatgacatggttgatatcatcgcctacggtgacgaccttctggttggcacggattacgatctggacttcaatgaggtggcacgacgcgctgccaagttggggtataagatgactcctgccaacaagggttctgtcttccctccgacttcctctctttccgatgctgtttttctaaagcgcaaattcgtccaaaacaacgacggcttatacaaaccagttatggatttaaagaatttggaagccatgctctcctacttcaaaccaggaacactactcgagaagctgcaatctgtttctatgttggctcaacattctggaaaagaagaatatgatagattgatgcaccccttcgctgactacggtgccgtaccgagtcacgagtacctgcaggcaagatggagggccttgttcgactga

SEQ ID NO: 3 - DNA, 1686 bp, Senecavirus A
  misc_feature 1..1686: note = EFS, NM_005864.4
  source 1..1686: mol_type = genomic DNA
atggccattgccacgtcgacccagctggcccgggcactgtatgacaacaccgctgagtccccccaggagctgtccttccgccgaggggatgtcctacgggtcctgcagagagagggcgctggtggactggacggctggtgcctctgctccctacacggccagcagggcattgtgcccgccaacagggtgaagctcttgcctgctggcccagcacccaagcccagcctctctcctgcgtccccagcccagcctggctcaccatatccagccccagatcacagcaatgaggaccaggaggtgtatgtggtgccgcccccagctcggccctgtccaacctcaggacctccagctggaccttgcccaccctctcctgacctcatctacaaaatccccagagctagtgggacccagctggctgctcccagagatgccttggaggtctacgatgtgccccccaccgccctccgggtgccctccagtggcccctatgactgccctgcctccttttcccaccctctgacccgggttgccccgcagccccctggagaggatgatgctccctatgatgtgcctctgaccccaaagccacctgcagagctggaaccagatctggagtgggaaggaggccgggagccggggccccccatctatgctgccccctccaacctgaaacgagcgtcagccttactcaatttgtatgaagcacccgaggaactgctggcagacggggagggcgggggcactgatgaggggatctacgatgtgcctctgctggggccagaggctcccccttctccagagccccctggagccttggcctcccatgaccaggacaccctggcccagcttctggccagaagccccccacccccacacaggccccggctcccctcagctgagagcctgtcccgccgccctctgcctgccctgcctgtccctgaggcccccagcccctccccagtgccctctcctgccccaggccggaagggcagcatccaggaccggcctctgcccccacccccaccccgcctgcctggttatggaggccccaaggtcgagggggatccagagggcagggagatggaggatgacccagcaggacaccacaatgagtacgagggcattccgatggccgaggagtatgactatgtccacctgaagggcatggacaaagctcagggatctaggcccccggatcaggcctgcacaggggatcctgaactgcccgagagggggatgccggcgccgcaggaggccctgtccccaggggagccactggttgtgtccaccggagatctgcagctcctgtacttctatgctgggcaatgccagagccactactcagccctgcaggcagccgtggcagccctgatgtccagtacccaggctaatcagcccccgcgccttttcgtgccccacagcaagagggtggtggtggctgctcatcgcctggtgtttgttggggacaccctgggccggctggcagcctctgcccctctgagagcacaggtcagggctgcaggtacagcactgggccaggcattgcgggccactgtgctggctgtcaagggagctgccctgggctacccatccagccctgccatccaagagatggtgcagtgtgtaacagaactggcagggcaggccctgcaattcactaccctgctcactagcctggctccatga

SEQ ID NO: 4 - protein, 561 aa, Senecavirus A
  REGION 1..561: note = misc_feature - EFS, NM_005864.4
MAIATSTQLARALYDNTAESPQELSFRRGDVLRVLQREGAGGLDGWCLCSLHGQQGIVPANRVKLLPAGPAPKPSLSPASPAQPGSPYPAPDHSNEDQEVYVVPPPARPCPTSGPPAGPCPPSPDLIYKIPRASGTQLAAPRDALEVYDVPPTALRVPSSGPYDCPASFSHPLTRVAPQPPGEDDAPYDVPLTPKPPAELEPDLEWEGGREPGPPIYAAPSNLKRASALLNLYEAPEELLADGEGGGTDEGIYDVPLLGPEAPPSPEPPGALASHDQDTLAQLLARSPPPPHRPRLPSAESLSRRPLPALPVPEAPSPSPVPSPAPGRKGSIQDRPLPPPPPRLPGYGGPKVEGDPEGREMEDDPAGHHNEYEGIPMAEEYDYVHLKGMDKAQGSRPPDQACTGDPELPERGMPAPQEALSPGEPLVVSTGDLQLLYFYAGQCQSHYSALQAAVAALMSSTQANQPPRLFVPHSKRVVVAAHRLVFVGDTLGRLAASAPLRAQVRAAGTALGQALRATVLAVKGAALGYPSSPAIQEMVQCVTELAGQALQFTTLLTSLAP

SEQ ID NO: 5 - DNA, 768 bp, Senecavirus A
  misc_feature 1..768: note = MAFP4, NM_002404.3
  source 1..768: mol_type = genomic DNA
atgaaggcactcctggccctgccgctgctgctgcttctctccacgcccccgtgtgccccccaggtctccgggatccgaggagatgctctggagaggttttgccttcagcaacccctggactgtgacgacatctatgcccagggctaccagtcagacggcgtgtacctcatctacccctcgggccccagtgtgcctgtgcccgtcttctgtgacatgaccaccgagggcgggaagtggacggttttccagaagagattcaatggctcagtaagtttcttccgcggctggaatgactacaagctgggcttcggccgtgctgatggagagtactggctggggctgcagaacatgcacctcctgacactgaagcagaagtatgagctgcgagtggacttggaggactttgagaacaacacggcctatgccaagtacgctgacttctccatctccccgaacgcggtcagcgcagaggaggatggctacaccctctttgtggcaggctttgaggatggcggggcaggtgactccctgtcctaccacagtggccagaagttctctaccttcgaccgggaccaggacctctttgtgcagaactgcgcagctctctcctcaggagccttctggttccgcagctgccactttgccaacctcaatggcttctacctaggtggctcccacctctcttatgccaatggcatcaactgggcccagtggaagggcttctactactccctcaaacgcactgagatgaaaatccgccgggcctga

SEQ ID NO: 6 - protein, 255 aa, Senecavirus A
  REGION 1..255: note = misc_feature - MAFP4, NM_002404.3
MKALLALPLLLLLSTPPCAPQVSGIRGDALERFCLQQPLDCDDIYAQGYQSDGVYLIYPSGPSVPVPVFCDMTTEGGKWTVFQKRFNGSVSFFRGWNDYKLGFGRADGEYWLGLQNMHLLTLKQKYELRVDLEDFENNTAYAKYADFSISPNAVSAEEDGYTLFVAGFEDGGAGDSLSYHSGQKFSTFDRDQDLFVQNCAALSSGAFWFRSCHFANLNGFYLGGSHLSYANGINWAQWKGFYYSLKRTEMKIRRA

SEQ ID NO: 7 - DNA, 1743 bp, Senecavirus A
  misc_feature 1..1743: note = NTN3, NM_006181.3
  source 1..1743: mol_type = genomic DNA
atgcctggctggccctgggggctgctgctgacggcaggcacgctcttcgccgccctgagtcctgggccgccggcgcccgccgacccctgccacgatgaggggggtgcgccccgcggctgcgtgccaggactggtgaacgccgccctgggccgcgaggtgctggcttccagcacgtgcgggcggccggccactcgggcctgcgacgcctccgacccgcgacgggcacactcccccgccctccttacttccccagggggcacggccagccctctgtgctggcgctcggagtccctgcctcgggcgcccctcaacgtgactctcacggtgcccctgggcaaggcttttgagctggtcttcgtgagcctgcgcttctgctcagctcccccagcctccgtggccctgctcaagtctcaggaccatggccgcagctgggccccgctgggcttcttctcctcccactgtgacctggactatggccgtctgcctgcccctgccaatggcccagctggcccagggcctgaggccctgtgcttccccgcacccctggcccagcctgatggcagcggccttctggccttcagcatgcaggacagcagccccccaggcctggacctggacagcagcccagtgctccaagactgggtgaccgccaccgacgtccgtgtagtgctcacaaggcctagcacggcaggtgaccccagggacatggaggccgtcgtcccttactcctacgcagccaccgacctccaggtgggcgggcgctgcaagtgcaatggacatgcctcacggtgcctgctggacacacagggccacctgatctgcgactgtcggcatggcaccgagggccctgactgcggccgctgcaagcccttctactgcgacaggccatggcagcgggccactgcccgggaatcccacgcctgcctcgcttgctcctgcaacggccatgcccgccgctgccgcttcaacatggagctgtaccgactgtccggccgccgcagcgggggtgtctgtctcaactgccggcacaacaccgccggccgccactgccactactgccgggagggcttctatcgagaccctggccgtgccctgagtgaccgtcgggcttgcagggcctgcgactgtcacccggttggtgctgctggcaagacctgcaaccagaccacaggccagtgtccctgcaaggatggcgtcactggcctcacctgcaaccgctgcgcgcctggcttccagcaaagccgctccccagtggcgccctgtgttaagacccctatccctggacccactgaggacagcagccctgtgcagccccaggactgtgactcgcactgcaaacctgcccgtggcagctaccgcatcagcctaaagaagttctgcaagaaggactatgcggtgcaggtggcggtgggtgcgcgcggcgaggcgcgcggcgcgtggacacgcttcccggtggcggtgctcgccgtgttccggagcggagaggagcgcgcgcggcgcgggagtagcgcgctgtgggtgcccgccggggatgcggcctgcggctgcccgcgcctgctccccggccgccgctacctcctgctggggggcgggcctggagccgcggctgggggcgcggggggccgggggcccgggctcatcgccgcccgcggaagcctcgtgctaccctggagggacgcgtggacgcggcgcctgcggaggctgcagcgacgcgaacggcgggggcgctgcagcgccgcctga

SEQ ID NO: 8 - protein, 580 aa, Senecavirus A
  REGION 1..580: note = misc_feature - NTN3, NM_006181.3
MPGWPWGLLLTAGTLFAALSPGPPAPADPCHDEGGAPRGCVPGLVNAALGREVLASSTCGRPATRACDASDPRRAHSPALLTSPGGTASPLCWRSESLPRAPLNVTLTVPLGKAFELVFVSLRFCSAPPASVALLKSQDHGRSWAPLGFFSSHCDLDYGRLPAPANGPAGPGPEALCFPAPLAQPDGSGLLAFSMQDSSPPGLDLDSSPVLQDWVTATDVRVVLTRPSTAGDPRDMEAVVPYSYAATDLQVGGRCKCNGHASRCLLDTQGHLICDCRHGTEGPDCGRCKPFYCDRPWQRATARESHACLACSCNGHARRCRFNMELYRLSGRRSGGVCLNCRHNTAGRHCHYCREGFYRDPGRALSDRRACRACDCHPVGAAGKTCNQTTGQCPCKDGVTGLTCNRCAPGFQQSRSPVAPCVKTPIPGPTEDSSPVQPQDCDSHCKPARGSYRISLKKFCKKDYAVQVAVGARGEARGAWTRFPVAVLAVFRSGEERARRGSSALWVPAGDAACGCPRLLPGRRYLLLGGGPGAAAGGAGGRGPGLIAARGSLVLPWRDAWTRRLRRLQRRERRGRCSAA

SEQ ID NO: 9 - DNA, 1326 bp, Senecavirus A
  misc_feature 1..1326: note = SOX11, NM_003108.4
  source 1..1326: mol_type = genomic DNA
atggtgcagcaggcggagagcttggaagcggagagcaacctgccccgggaggcgctggacacggaggagggcgaattcatggcttgcagcccggtggccctggacgagagcgacccagactggtgcaagacggcgtcgggccacatcaagcggccgatgaacgcgttcatggtatggtccaagatcgaacgcaggaagatcatggagcagtctccggacatgcacaacgccgagatctccaagaggctgggcaagcgctggaaaatgctgaaggacagcgagaagatcccgttcatccgggaggcggagcggctgcggctcaagcacatggccgactaccccgactacaagtaccggccccggaaaaagcccaaaatggacccctcggccaagcccagcgccagccagagcccagagaagagcgcggccggcggcggcggcgggagcgcgggcggaggcgcgggcggtgccaagacctccaagggctccagcaagaaatgcggcaagctcaaggcccccgcggccgcgggcgccaaggcgggcgcgggcaaggcggcccagtccggggactacgggggcgcgggcgacgactacgtgctgggcagcctgcgcgtgagcggctcgggcggcggcggcgcgggcaagacggtcaagtgcgtgtttctggatgaggacgacgacgacgacgacgacgacgacgagctgcagctgcagatcaaacaggagccggacgaggaggacgaggaaccaccgcaccagcagctcctgcagccgccggggcagcagccgtcgcagctgctgagacgctacaacgtcgccaaagtgcccgccagccctacgctgagcagctcggcggagtcccccgagggagcgagcctctacgacgaggtgcgggccggcgcgacctcgggcgccgggggcggcagccgcctctactacagcttcaagaacatcaccaagcagcacccgccgccgctcgcgcagcccgcgctgtcgcccgcgtcctcgcgctcggtgtccacctcctcgtccagcagcagcggcagcagcagcggcagcagcggcgaggacgccgacgacctgatgttcgacctgagcttgaatttctctcaaagcgcgcacagcgccagcgagcagcagctggggggcggcgcggcggccgggaacctgtccctgtcgctggtggataaggatttggattcgttcagcgagggcagcctgggctcccacttcgagttccccgactactgcacgccggagctgagcgagatgatcgcgggggactggctggaggcgaacttctccgacctggtgttcacatattga

SEQ ID NO: 10 - protein, 441 aa, Senecavirus A
  REGION 1..441: note = misc_feature - SOX11, NM_003108.4
MVQQAESLEAESNLPREALDTEEGEFMACSPVALDESDPDWCKTASGHIKRPMNAFMVWSKIERRKIMEQSPDMHNAEISKRLGKRWKMLKDSEKIPFIREAERLRLKHMADYPDYKYRPRKKPKMDPSAKPSASQSPEKSAAGGGGGSAGGGAGGAKTSKGSSKKCGKLKAPAAAGAKAGAGKAAQSGDYGGAGDDYVLGSLRVSGSGGGGAGKTVKCVFLDEDDDDDDDDDELQLQIKQEPDEEDEEPPHQQLLQPPGQQPSQLLRRYNVAKVPASPTLSSSAESPEGASLYDEVRAGATSGAGGGSRLYYSFKNITKQHPPPLAQPALSPASSRSVSTSSSSSSGSSSGSSGEDADDLMFDLSLNFSQSAHSASEQQLGGGAAAGNLSLSLVDKDLDSFSEGSLGSHFEFPDYCTPELSEMIAGDWLEANFSDLVFTY

SEQ ID NO: 11 - DNA, 3063 bp, Senecavirus A
  misc_feature 1..3063: note = NEFH, NM_021076.4
  source 1..3063: mol_type = genomic DNA
atgatgagcttcggcggcgcggacgcgctgctgggcgccccgttcgcgccgctgcatggcggcggcagcctccactacgcgctagcccgaaagggtggcgcaggcgggacgcgctccgccgctggctcctccagcggcttccactcgtggacacggacgtccgtgagctccgtgtccgcctcgcccagccgcttccgtggcgcaggcgccgcctcaagcaccgactcgctggacacgctgagcaacgggccggagggctgcatggtggcggtggccacctcacgcagtgagaaggagcagctgcaggcgctgaacgaccgcttcgccgggtacatcgacaaggtgcggcagctggaggcgcacaaccgcagcctggagggcgaggctgcggcgctgcggcagcagcaggcgggccgctccgctatgggcgagctgtacgagcgcgaggtccgcgagatgcgcggcgcggtgctgcgcctgggcgcggcgcgcggtcagctacgcctggagcaggagcacctgctcgaggacatcgcgcacgtgcgccagcgcctagacgacgaggcccggcagcgagaggaggccgaggcggcggcccgcgcgctggcgcgcttcgcgcaggaggccgaggcggcgcgcgtggacctgcagaagaaggcgcaggcgctgcaggaggagtgcggctacctgcggcgccaccaccaggaagaggtgggcgagctgctcggccagatccagggctccggcgccgcgcaggcgcagatgcaggccgagacgcgcgacgccctgaagtgcgacgtgacgtcggcgctgcgcgagattcgcgcgcagcttgaaggccacgcggtgcagagcacgctgcagtccgaggagtggttccgagtgaggctggaccgactgtcggaggcagccaaggtgaacacagacgctatgcgctcagcgcaggaggagataactgagtaccggcgtcagctgcaggccaggaccacagagctggaggcactgaaaagcaccaaggactcactggagaggcagcgctctgagctggaggaccgtcatcaggccgacattgcctcctaccaggaagccattcagcagctggacgctgagctgaggaacaccaagtgggagatggccgcccagctgcgagaataccaggacctgctcaatgtcaagatggctctggatatagagatagccgcttacagaaaactcctggaaggtgaagagtgtcggattggctttggcccaattcctttctcgcttccagaaggactccccaaaattccctctgtgtccactcacataaaggtgaaaagcgaagagaagatcaaagtggtggagaagtctgagaaagaaactgtgattgtggaggaacagacagaggagacccaagtgactgaagaagtgactgaagaagaggagaaagaggccaaagaggaggagggcaaggaggaagaagggggtgaagaagaggaggcagaagggggagaagaagaaacaaagtctcccccagcagaagaggctgcatccccagagaaggaagccaagtcaccagtaaaggaagaggcaaagtcaccggctgaggccaagtccccagagaaggaggaagcaaaatccccagccgaagtcaagtcccctgagaaggccaagtctccagcaaaggaagaggcaaagtcaccgcctgaggccaagtccccagagaaggaggaagcaaaatctccagctgaggtcaagtcccccgagaaggccaagtccccagcaaaggaagaggcaaagtcaccggctgaggccaagtctccagagaaggccaagtccccagtgaaggaagaagcaaagtcaccggctgaggccaagtccccagtgaaggaagaagcaaaatctccagctgaggtcaagtccccggaaaaggccaagtctccaacgaaggaggaagcaaagtcccctgagaaggccaagtccccagagaaggaagaggccaagtcccctgagaaggccaagtccccagtgaaggcagaagcaaagtcccctgagaaggccaagtccccagtgaaggcagaagcaaagtcccctgagaaggccaagtccccagtgaaggaagaagcaaagtcccctgagaaggccaagtccccagtgaaggaagaagcaaagtcccctgagaaggccaagtccccagtgaaggaagaagcaaagacccccgagaaggccaagtccccagtgaaggaagaagctaagtccccagagaaggccaagtccccagagaaggccaagactcttgatgtgaagtctccagaagccaagactccagcgaaggaggaagcaaggtcccctgcagacaaattccctgaaaaggccaaaagccctgtcaaggaggaggtcaagtccccagagaaggcgaaatctcccctgaaggaggatgccaaggcccctgagaaggagatcccaaaaaaggaagaggtgaagtccccagtgaaggaggaggagaagccccaggaggtgaaagtcaaagagcccccaaagaaggcagaggaagagaaagcccctgccacaccaaaaacagaggagaagaaggacagcaagaaagaggaggcacccaagaaggaggctccaaagcccaaggtggaggagaagaaggaacctgctgtcgaaaagcccaaagaatccaaagttgaagccaagaaggaagaggctgaagataagaaaaaagtccccaccccagagaaggaggctcctgccaaggtggaggtgaaggaagacgctaaacccaaagaaaagacagaggtagccaagaaggaaccagatgatgccaaggccaaggaacccagcaaaccagcagagaagaaggaggcagcaccggagaaaaaagacaccaaggaggagaaggccaagaagcctgaggagaaacccaagacagaggccaaagccaaggaagatgacaagaccctctcaaaagagcctagcaagcctaaggcagaaaaggctgaaaaatcctccagcacagaccaaaaagacagcaagcctccagagaaggccacagaagacaaggccgccaaggggaagtaa

SEQ ID NO: 12 - protein, 1020 aa, Senecavirus A
  REGION 1..1020: note = misc_feature - NEFH, NM_021076.4
MMSFGGADALLGAPFAPLHGGGSLHYALARKGGAGGTRSAAGSSSGFHSWTRTSVSSVSASPSRFRGAGAASSTDSLDTLSNGPEGCMVAVATSRSEKEQLQALNDRFAGYIDKVRQLEAHNRSLEGEAAALRQQQAGRSAMGELYEREVREMRGAVLRLGAARGQLRLEQEHLLEDIAHVRQRLDDEARQREEAEAAARALARFAQEAEAARVDLQKKAQALQEECGYLRRHHQEEVGELLGQIQGSGAAQAQMQAETRDALKCDVTSALREIRAQLEGHAVQSTLQSEEWFRVRLDRLSEAAKVNTDAMRSAQEEITEYRRQLQARTTELEALKSTKDSLERQRSELEDRHQADIASYQEAIQQLDAELRNTKWEMAAQLREYQDLLNVKMALDIEIAAYRKLLEGEECRIGFGPIPFSLPEGLPKIPSVSTHIKVKSEEKIKVVEKSEKETVIVEEQTEETQVTEEVTEEEEKEAKEEEGKEEEGGEEEEAEGGEEETKSPPAEEAASPEKEAKSPVKEEAKSPAEAKSPEKEEAKSPAEVKSPEKAKSPAKEEAKSPPEAKSPEKEEAKSPAEVKSPEKAKSPAKEEAKSPAEAKSPEKAKSPVKEEAKSPAEAKSPVKEEAKSPAEVKSPEKAKSPTKEEAKSPEKAKSPEKEEAKSPEKAKSPVKAEAKSPEKAKSPVKAEAKSPEKAKSPVKEEAKSPEKAKSPVKEEAKSPEKAKSPVKEEAKTPEKAKSPVKEEAKSPEKAKSPEKAKTLDVKSPEAKTPAKEEARSPADKFPEKAKSPVKEEVKSPEKAKSPLKEDAKAPEKEIPKKEEVKSPVKEEEKPQEVKVKEPPKKAEEEKAPATPKTEEKKDSKKEEAPKKEAPKPKVEEKKEPAVEKPKESKVEAKKEEAEDKKKVPTPEKEAPAKVEVKEDAKPKEKTEVAKKEPDDAKAKEPSKPAEKKEAAPEKKDTKEEKAKKPEEKPKTEAKAKEDDKTLSKEPSKPKAEKAEKSSSTDQKDSKPPEKATEDKAAKGK

SEQ ID NO: 13 - DNA, 1119 bp, Senecavirus A
  misc_feature 1..1119: note = CNTFR, NM_147164.3
  source 1..1119: mol_type = genomic DNA
atggctgctcctgtcccgtgggcctgctgtgctgtgcttgccgccgccgccgcagttgtctacgcccagagacacagtccacaggaggcaccccatgtgcagtacgagcgcctgggctctgacgtgacactgccatgtgggacagcaaactgggatgctgcggtgacgtggcgggtaaatgggacagacctggcccctgacctgctcaacggctctcagctggtgctccatggcctggaactgggccacagtggcctctacgcctgcttccaccgtgactcctggcacctgcgccaccaagtcctgctgcatgtgggcttgccgccgcgggagcctgtgctcagctgccgctccaacacttaccccaagggcttctactgcagctggcatctgcccacccccacctacattcccaacaccttcaatgtgactgtgctgcatggctccaaaattatggtctgtgagaaggacccagccctcaagaaccgctgccacattcgctacatgcacctgttctccaccatcaagtacaaggtctccataagtgtcagcaatgccctgggccacaatgccacagctatcacctttgacgagttcaccattgtgaagcctgatcctccagaaaatgtggtagcccggccagtgcccagcaaccctcgccggctggaggtgacgtggcagaccccctcgacctggcctgaccctgagtcttttcctctcaagttctttctgcgctaccgacccctcatcctggaccagtggcagcatgtggagctgtccgacggcacagcacacaccatcacagatgcctacgccgggaaggagtacattatccaggtggcagccaaggacaatgagattgggacatggagtgactggagcgtagccgcccacgctacgccctggactgaggaaccgcgacacctcaccacggaggcccaggctgcggagaccacgaccagcaccaccagctccctggcacccccacctaccacgaagatctgtgaccctggggagctgggcagcggcgggggaccctcggcacccttcttggtcagcgtccccatcactctggccctggctgccgctgccgccactgccagcagtctcttgatctga

SEQ ID NO: 14 - protein, 372 aa, Senecavirus A
  REGION 1..372: note = misc_feature - CNTFR, NM_147164.3
MAAPVPWACCAVLAAAAAVVYAQRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSGGGPSAPFLVSVPITLALAAAAATASSLLI

SEQ ID NO: 15 - DNA, 1887 bp, Senecavirus A
  misc_feature 1..1887: note = JPH4, NM_001146028.2
  source 1..1887: mol_type = genomic DNA
atgtcccccgggggcaagttcgactttgacgacgggggctgctacgtggggggctgggaggcggggcgggcacatggctacggcgtgtgcacgggccccggcgcccagggcgagtacagcggctgctgggcacacggcttcgagtcactgggcgtcttcacggggcccggcggacacagctaccagggccactggcagcagggcaagcgcgaagggctgggcgtggagcgcaagagccgctggacgtaccgcggcgagtggctgggcgggctgaaggggcgcagcggcgtgtgggaaagcgtgtccggcctgcgctacgccgggctctggaaggacggtttccaggacggctacggcactgagacctactccgacggaggcacctaccagggccagtggcaggccgggaagcgccacggctacggggtacgccagagtgtgccctaccatcaggcggcgctgctgcgctcgccccgccgcacctccctggattccggccacagcgaccccccgacgccacccccgcccctgcccttgccgggcgacgagggaggcagccccgcctcgggctcccggggcggcttcgtgctggccgggcccggggacgccgacggcgcgtcgtcccgaaagcgcactccggcggccggcggattctttcgccgttcgctgctgctcagcgggctccgagcgggcggacgtcgcagctccctgggcagcaagcgaggctccctgcgcagcgaggtgagcagcgaggtgggcagcaccggaccgcccggctcggaggccagcgggcccccggccgcagcgccgcccgccctcatcgagggctcggccacagaggtgtacgcgggcgagtggcgcgcagatcggcgcagcggcttcggcgtcagccagcgctccaacgggctgcgctacgagggcgagtggctgggcaaccggcggcacggctacgggcgcaccacccgccccgacggctcccgcgaggagggcaagtacaagcgcaaccggctggtgcacggcgggcgcgtccgcagtctcctgcctctggcccttcggcggggcaaggttaaggagaaggtggacagggctgtcgagggcgcccgtcgagccgtgagtgctgcccgtcagcgccaggagatcgccgctgccagggcagcagacgccctcctaaaggcagtggcagccagcagtgtcgctgagaaggccgtggaggcagctcgaatggccaaactgatagcccaggacctgcagcccatgctagaggccccaggccgcagacccaggcaggactcagaaggttccgacacggagcccctggatgaggacagccctggggtatatgagaacggactgaccccctcagagggatcccctgaactgcccagcagtcctgcctcctcccgccaaccctggcgaccccctgcctgccggagcccactgcctcctggaggggaccagggtcccttctccagccccaaagcttggcctgaggagtgggggggggcaggcgcacaggcagaggaactagctggctatgaggctgaggatgaggctgggatgcaagggccagggcccagagacggttccccactcctcggaggctgcagcgacagttcaggaagtcttcgagaggaggagggggaggatgaagagcccctgcccccgctgagggccccagcaggcacggagcctgagcccatcgccatgctggtcctgaggggctcgtcctcgaggggtcctgatgctgggtgcctgacagaagagctcggggagcccgctgcaaccgagaggcctgcccagccgggagctgccaaccccctggtggtgggagccgtggccctcctggacctcagcctggcattcctgttctcccagctcctcacctga

SEQ ID NO: 16 - protein, 628 aa, Senecavirus A
  REGION 1..628: note = misc_feature - JPH4, NM_001146028.2
MSPGGKFDFDDGGCYVGGWEAGRAHGYGVCTGPGAQGEYSGCWAHGFESLGVFTGPGGHSYQGHWQQGKREGLGVERKSRWTYRGEWLGGLKGRSGVWESVSGLRYAGLWKDGFQDGYGTETYSDGGTYQGQWQAGKRHGYGVRQSVPYHQAALLRSPRRTSLDSGHSDPPTPPPPLPLPGDEGGSPASGSRGGFVLAGPGDADGASSRKRTPAAGGFFRRSLLLSGLRAGGRRSSLGSKRGSLRSEVSSEVGSTGPPGSEASGPPAAAPPALIEGSATEVYAGEWRADRRSGFGVSQRSNGLRYEGEWLGNRRHGYGRTTRPDGSREEGKYKRNRLVHGGRVRSLLPLALRRGKVKEKVDRAVEGARRAVSAARQRQEIAAARAADALLKAVAASSVAEKAVEAARMAKLIAQDLQPMLEAPGRRPRQDSEGSDTEPLDEDSPGVYENGLTPSEGSPELPSSPASSRQPWRPPACRSPLPPGGDQGPFSSPKAWPEEWGGAGAQAEELAGYEAEDEAGMQGPGPRDGSPLLGGCSDSSGSLREEEGEDEEPLPPLRAPAGTEPEPIAMLVLRGSSSRGPDAGCLTEELGEPAATERPAQPGAANPLVVGAVALLDLSLAFLFSQLLT

SEQ ID NO: 17 - DNA, 1695 bp, Senecavirus A
  misc_feature 1..1695: note = ANTXR1, NM_032208.3
  source 1..1695: mol_type = genomic DNA
atggccacggcggagcggagagccctcggcatcggcttccagtggctctctttggccactctggtgctcatctgcgccgggcaagggggacgcagggaggatgggggtccagcctgctacggcggatttgacctgtacttcattttggacaaatcaggaagtgtgctgcaccactggaatgaaatctattactttgtggaacagttggctcacaaattcatcagcccacagttgagaatgtcctttattgttttctccacccgaggaacaaccttaatgaaactgacagaagacagagaacaaatccgtcaaggcctagaagaactccagaaagttctgccaggaggagacacttacatgcatgaaggatttgaaagggccagtgagcagatttattatgaaaacagacaagggtacaggacagccagcgtcatcattgctttgactgatggagaactccatgaagatctctttttctattcagagagggaggctaataggtctcgagatcttggtgcaattgtttactgtgttggtgtgaaagatttcaatgagacacagctggcccggattgcggacagtaaggatcatgtgtttcccgtgaatgacggctttcaggctctgcaaggcatcatccactcaattttgaagaagtcctgcatcgaaattctagcagctgaaccatccaccatatgtgcaggagagtcatttcaagttgtcgtgagaggaaacggcttccgacatgcccgcaacgtggacagggtcctctgcagcttcaagatcaatgactcggtcacactcaatgagaagcccttttctgtggaagatacttatttactgtgtccagcgcctatcttaaaagaagttggcatgaaagctgcactccaggtcagcatgaacgatggcctctcttttatctccagttctgtcatcatcaccaccacacactgttctgacggttccatcctggccatcgccctgctgatcctgttcctgctcctagccctggctctcctctggtggttctggcccctctgctgcactgtgattatcaaggaggtccctccaccccctgccgaggagagtgaggaagaagatgatgatggtctgcctaagaaaaagtggccaacggtagacgcctcttattatggtgggagaggcgttggaggcattaaaagaatggaggttcgttggggagaaaagggctccacagaagaaggtgctaagttggaaaaggcaaagaatgcaagagtcaagatgccggagcaggaatatgaattccctgagccgcgaaatctcaacaacaatatgcgtcggccttcttccccccggaagtggtactctccaatcaagggaaaactcgatgccttgtgggtcctactgaggaaaggatatgatcgtgtgtctgtgatgcgtccacagccaggagacacggggcgctgcatcaacttcaccagggtcaagaacaaccagccagccaagtacccactcaacaacgcctaccacacctcctcgccgcctcctgcccccatctacactcccccacctcctgcgccccactgccctcccccgccccccagcgcccctacccctcccatcccgtccccaccttccacccttccccctcctccccaggctccacctcccaacagggcacctcctccctcccgccctcctccaaggccttctgtctag

SEQ ID NO: 18 - protein, 564 aa, Senecavirus A
  REGION 1..564: note = misc_feature - ANTXR1, NM_032208.3
MATAERRALGIGFQWLSLATLVLICAGQGGRREDGGPACYGGFDLYFILDKSGSVLHHWNEIYYFVEQLAHKFISPQLRMSFIVFSTRGTTLMKLTEDREQIRQGLEELQKVLPGGDTYMHEGFERASEQIYYENRQGYRTASVIIALTDGELHEDLFFYSEREANRSRDLGAIVYCVGVKDFNETQLARIADSKDHVFPVNDGFQALQGIIHSILKKSCIEILAAEPSTICAGESFQVVVRGNGFRHARNVDRVLCSFKINDSVTLNEKPFSVEDTYLLCPAPILKEVGMKAALQVSMNDGLSFISSSVIITTTHCSDGSILAIALLILFLLLALALLWWFWPLCCTVIIKEVPPPPAEESEEEDDDGLPKKKWPTVDASYYGGRGVGGIKRMEVRWGEKGSTEEGAKLEKAKNARVKMPEQEYEFPEPRNLNNNMRRPSSPRKWYSPIKGKLDALWVLLRKGYDRVSVMRPQPGDTGRCINFTRVKNNQPAKYPLNNAYHTSSPPPAPIYTPPPPAPHCPPPPPSAPTPPIPSPPSTLPPPPQAPPPNRAPPPSRPPPRPSV

SEQ ID NO: 19 - DNA, 954 bp, Senecavirus A
  misc_feature 1..954: note = RPLP0, NM_001002.4
  source 1..954: mol_type = genomic DNA
atgcccagggaagacagggcgacctggaagtccaactacttccttaagatcatccaactattggatgattatccgaaatgtttcattgtgggagcagacaatgtgggctccaagcagatgcagcagatccgcatgtcccttcgcgggaaggctgtggtgctgatgggcaagaacaccatgatgcgcaaggccatccgagggcacctggaaaacaacccagctctggagaaactgctgcctcatatccgggggaatgtgggctttgtgttcaccaaggaggacctcactgagatcagggacatgttgctggccaataaggtgccagctgctgcccgtgctggtgccattgccccatgtgaagtcactgtgccagcccagaacactggtctcgggcccgagaagacctcctttttccaggctttaggtatcaccactaaaatctccaggggcaccattgaaatcctgagtgatgtgcagctgatcaagactggagacaaagtgggagccagcgaagccacgctgctgaacatgctcaacatctcccccttctcctttgggctggtcatccagcaggtgttcgacaatggcagcatctacaaccctgaagtgcttgatatcacagaggaaactctgcattctcgcttcctggagggtgtccgcaatgttgccagtgtctgtctgcagattggctacccaactgttgcatcagtaccccattctatcatcaacgggtacaaacgagtcctggccttgtctgtggagacggattacaccttcccacttgctgaaaaggtcaaggccttcttggctgatccatctgcctttgtggctgctgcccctgtggctgctgccaccacagctgctcctgctgctgctgcagccccagctaaggttgaagccaaggaagagtcggaggagtcggacgaggatatgggatttggtctctttgactaa

SEQ ID NO: 20 - protein, 317 aa, Senecavirus A
  REGION 1..317: note = misc_feature - RPLP0, NM_001002.4
MPREDRATWKSNYFLKIIQLLDDYPKCFIVGADNVGSKQMQQIRMSLRGKAVVLMGKNTMMRKAIRGHLENNPALEKLLPHIRGNVGFVFTKEDLTEIRDMLLANKVPAAARAGAIAPCEVTVPAQNTGLGPEKTSFFQALGITTKISRGTIEILSDVQLIKTGDKVGASEATLLNMLNISPFSFGLVIQQVFDNGSIYNPEVLDITEETLHSRFLEGVRNVASVCLQIGYPTVASVPHSIINGYKRVLALSVETDYTFPLAEKVKAFLADPSAFVAAAPVAAATTAAPAAAAAPAKVEAKEESEESDEDMGFGLFD

(19) United States
(12) Patent Application Publication
(10) Pub. No.: US 2026/0338797 A1
(43) Pub. Date: Oct. 29, 2026

(54) CANCER THERAPIES WITH ONCOLYTIC VIRUS AND AN IMMUNE CHECKPOINT INHIBITOR

(71) Applicant: SENECA THERAPEUTICS, INC., Blue Bell, PA (US)

(72) Inventors: Paul L. HALLENBECK, Blue Bell, PA (US); Sunil CHADA, Blue Bell, PA (US); Lina S. FRANCO ACHURY, Phoenix, AZ (US)

(73) Assignee: SENECA THERAPEUTICS, INC., Blue Bell, PA (US)

(21) Appl. No.: 19/109,726

(22) PCT Filed: Sep. 8, 2023
(86) PCT No.: PCT/US2023/073767

§ 371 Date: Mar. 7, 2025

Related U.S. Application Data
(60) Provisional application No. 63/375,049, filed on Sep. 8, 2022.

(51) Int. Cl. G01N 33/68; C12Q 1/6886

(52) U.S. Cl. CPC G01N 33/6893; C12Q 1/6886; C12Q 2600/158; G01N 2333/705; G01N 2800/52

(57) ABSTRACT

Provided herein are biomarkers that may be used to identify a cancer that is susceptible to treatment with Seneca Valley Virus (SVV) or SVV derivative. The disclosure also relates to in vitro screening methods for cancers that are susceptible to treatment with SVV as well as methods of treating such cancers. Also provided herein are kits containing agents that may be used to screen for the biomarkers. Also disclosed methods of using the biomarkers to identify and treat poorly differentiated neuroendocrine carcinomas (NECs) and well-differentiated neuroendocrine tumors (NETs) with SVV-001 in combination with the programmed death receptor 1 (PD-1) immune checkpoint blocker (ICB: nivolumab).

### CROSS-REFERENCE TO RELATED APPLICATIONS

This application claims priority to U.S. Provisional Application No. 63/375,049, which was filed on Sep. 8, 2022, the content of which is hereby incorporated by reference in its entirety.

### SEQUENCE LISTING

The instant application contains a Sequence Listing which is being submitted herewith electronically in XML format and is hereby incorporated by reference in its entirety. Said XML copy, created on Sep. 8, 2023, is named 115029_000083_SequenceListing.xml and is 58,657 bytes in size.

### TECHNICAL FIELD

Disclosed herein are compositions and methods for treating cancer. More particularly, the disclosed inventions relate to the field of treating cancer in a subject using an oncolytic virus, in particular Seneca Valley Virus (SVV). The disclosed inventions also relate to biomarkers that may be used to identify cancers susceptible to treatment with an oncolytic virus, in particular SVV or SVV derivative.

### BACKGROUND

Cancer is the second most common cause of death in the United States. One out of every four individuals dies from it, and more than one million new cancer diagnoses are made every year. The disease begins with the uncontrolled proliferation and growth of abnormal, transformed cells. However, the definition does not end with a description of one disease but of hundreds of different diseases. No two cancers are the same, nor are they clonal. The mutations driving cell changes and cell transformation may be similar, but they are often not identical. This conundrum adds to the complexity and heterogeneity of the pathologies that patients develop. Current cancer therapies, including chemotherapeutics and radiation, are most effective when combined with immunomodulatory agents to create and enhance the antitumor microenvironment. Many malignancies may be resistant to treatment via these traditional methods.

Oncolytic viruses show enormous potential as anti-cancer agents. The picornavirus Seneca Valley virus (SVV) is a single stranded (+) RNA virus that has been investigated as an oncolytic therapy. However, a significant number of tumors do not regress or demonstrate clinical benefit after infection leaving the patient searching for alternate treatment routes.

Accordingly, what is needed is a method that allows identification of the cancers that are susceptible to treatment with SVV. What is also needed is an improved therapeutic approach for using oncolytic viruses, in particular SVV, to treat cancers that have been identified as being susceptible to treatment with SVV.

### SUMMARY

Provided herein are improved methods, pharmaceutical compositions, and kits for treating cancer which use Seneca Valley Virus and an immune checkpoint inhibitor. Provided herein are also screening methods and kits that allow for identification of cancers that are susceptible to treatment with Seneca Valley Virus.

Disclosed herein are in vitro screening methods for identifying a cancer that is susceptible to treatment with SVV, the screening method comprises detecting the presence of one or more biomarkers in a cancer sample, wherein the presence of the one or more biomarkers is indicative of a cancer that may be treated with SVV and an immune checkpoint inhibitor. The screening method may include testing for ANTRX1. In one embodiment, the screening method relies on one or more biomarkers having increased or upregulated expression in cancers that are SVV-R. In other embodiments, the screening method relies on one or more biomarkers having increased or upregulated expression in cancers that are SVV-S. In certain embodiments, the one or more biomarkers include one or more or each of: EFS, MFAP4, NTN3, SOX11, NEFH, CNTFR, JPH4, and ANTXR1.

Further provided are methods of treating a cancer in a subject in need thereof comprising (1) screening the cancer for the presence of the one or more biomarkers indicative of a cancer that may be treated with SVV; and (2) administering to the subject an effective amount of Seneca Valley Virus and an immune checkpoint inhibitor when the one or more biomarkers is detected as expressed in the cancer.

Also provided herein are methods of treating a cancer in a subject in need thereof comprising administering to the subject an effective amount of SVV and an immune checkpoint inhibitor, wherein the cancer expresses one or more biomarkers indicative of a cancer that may be treated with SVV.

Further provided herein is a kit for use in an in vitro screening method for a cancer that is susceptible for treatment with SVV.

Other features and advantages of the invention will be apparent from the detailed description and examples that follow.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1A and FIG. 1B show flow charts generally outlining the process used to identify biomarkers that may be used to identify a cancer that can be treated with SVV. FIG. 1A shows Step 1 and Step 2. FIG. 1B shows Step 3.

FIG. 2 shows the expression of Panel A genes in SVV-R (resistant to treatment with SVV) and SVV-S(sensitive to treatment with SVV) cell lines.

FIG. 3 shows the results of RNAseq gene expression data from patients with small cell lung cancer (SCLC) compared against Panel A.

FIG. 4 shows the results RNA expression profiles from neuroendocrine carcinoma patients (NEC) compared against Panel A.

FIG. 5 shows the results of 69 PDX models compared against Panel A.

FIG. 6 shows the expression of Panel 2 genes in SVV-R and SVV-S cell lines including ANTRX1.

FIG. 7 shows the results of RNAseq gene expression data from patients with small cell lung cancer (SCLC) compared against Panel 2.

FIG. 8 shows the results RNA expression profiles from neuroendocrine carcinoma patients (NEC) compared against Panel 2.

FIG. 9 shows the results of 69 PDX models compared against Panel 2.

FIG. 10 shows the single sample Gene Set Enrichment Analysis (ssGSEA) for Panel 2 genes for the 69 PDX models. Correlation analysis between ssGSEA scores and probabilities of sensitivity based on the selected gene panel for 69 PDX models reveals high concordance (87%) on the prediction results. A-B: ssGSEA scores and probabilities of sensitivity based on the first 20-gene panel (gene set A); C-D: ssGSEA scores and probabilities of sensitivity based on new 20-gene panel containing EFS, NEFH and SOX11 genes (new_gene_set). Scatter plots showing the correlation between ssGSEA enrichment score and the predicted probability value are shown in the lower left boxes. Boxes with graph bars presented diagonally are ssGSEA score distribution for gene panel (Panel A, Panel B, Panel C, or Panel D) in PDX models by GSVA package. Spearman correlation coefficients are shown in top right boxes; the correlation value was calculated by Spearman method between the indicated panels, and statistical significance is represented by: * (p≤0.05), ** (p≤0.01), *** (p≤0.001).

FIG. 11 shows the results of testing for the expression of Panel A genes in organoid samples derived from normal tissues.

FIG. 12 shows the results of testing for the expression of Panel 2 genes in organoid samples derived from normal tissues.

FIG. 13 shows the results of gene expression of Panel 2.1 genes in SVV-R and SVV-S cell lines. Results are of a 5-gene panel (top 5 highly expressed genes from gene panel 2) across training set of cell lines. The prediction accuracy to distinguish SVV-Sensitive and SVV-Resistant cell lines was 81.78%.

FIG. 14 shows the results of gene expression of Panel 2.2 genes in SVV-R and SVV-S cell lines.

FIG. 15 shows the results of gene expression of Panel 3 genes in SVV-R and SVV-S cell lines.

FIG. 16 shows the results of gene expression of Panel 4 genes in SVV-R and SVV-S cell lines.

FIG. 17A shows ANTXR1 expression in sarcomas (Log2 ANTXR1 expression in 265 sarcoma patient samples from The Cancer Genome Atlas Program (TCGA) database).

FIG. 17B shows the probability for being SVV-R in sarcomas (probabilities of SVV-sensitivity based on the 20-gene panel A for 265 TCGA sarcoma patients. 94.7% (251/265) of sarcoma patients show probability of SVV-S>75%).

FIG. 17C shows the distribution of ANTRX1 and correlation to SVV-R. Top left panel shows a distribution of the predicted probability to be sensitive by a gene set in all sarcoma data. Lower left and top right panels show a scatter plot of the correlation, and the correlation value of 0.11, calculated by Spearman method. Lower right panels shows ANTXR1 expression (log2) distribution in all sarcoma data. Correlation analysis between ANTXR1 expression and probabilities of sensitivity based on the new 20-gene panel A for 265 TCGA sarcoma patients revealed no good correlation between ANTXR1 expression and the prediction results. Spearman correlation coefficient is shown, and statistical significance is represented by: * (p≤0.05), ** (p≤0.01), *** (p≤0.001).

FIG. 18 is a plot showing the probability of SVV sensitivity in 163 TCGA triple-negative breast cancer (TNBC) samples based on the 8 gene biomarker gene panel bioinformatics analysis. The bracket indicates the 103 out of 163 samples that are predicted to be SVV-Sensitive (63%) using 75% probability.

FIG. 19 is a graph showing assay reproducibility of the qRT-PCR assay for the 8-gene panel plus RPLPO, which was tested in NCI-H187 (SVV permissive) and H460 (SVV non-permissive) cells. The y-axis represents average Ct values. The first three bars per gene represent data for the NCI-H187 cell line obtained on days 1, 2, and 3 of the test. The last three bars per gene represent data for the H460 cell line obtained on days 1, 2, and 3 of the test.

FIG. 20 is a graph showing fold change (R-S) in expression of RPLPO, EFS, NTN3, MFAP, SOX11, NEFH, CNTFR, JPH4, and ANTXR1 genes in resistant (R) and sensitive(S) cell lines.

FIG. 21 is a diagram showing the trial schema (part I), and includes the following abbreviations: DLT=dose-limiting toxicity; MTD=maximum tolerated dose; NEC=neuroendocrine carcinoma; NET=neuroendocrine tumor; RP2D=recommended Phase 2 dose; VG=viral genomes.

FIG. 22 is an injection and biopsy schedule and includes the following abbreviations: IT=intratumoral(ly); IV=intravenous(ly).* All patients in Part I will have a pretreatment biopsy. Post-treatment biopsies will be completed for Cohort 6 on Day 15/29 and Day 29/43.

FIG. 23 is a graphical representation of the EFS gene expression in 19 different cell lines obtained with qRT-PCR (shown as thin line “qPCR” that represents Fold Change over RPLPO expression) and with RNAseq (shown as thick bars that represent Log2TPM). FIG. 23 demonstrates the strong correlation between the qPCR results and RNAseq results obtained for this gene across the 19 cell lines examined.

### DETAILED DESCRIPTION OF THE INVENTION

The general description and the following detailed description are exemplary and explanatory only and are not restrictive of the invention, as defined in the appended claims. Other aspects of the present invention will be apparent to those skilled in the art in view of the detailed description of the invention as provided herein.

The assays disclosed herein allow for the identification of cancer patients who will benefit from treatment with SVV either as monotherapy or in combination with other immune therapies like checkpoint inhibitors. The assays disclosed herein may also be used to help identify cancer patients that will benefit from treatment with armed SVV constructs.

The present invention relates to compositions and methods of using Seneca Valley Virus (SVV) for treating cancer in a subject. SVV is useful in a variety of applications such as treating a cancer, reducing, or inhibiting cancer cell growth, and increasing the survival of a subject suffering from cancer. The disclosed methods in certain embodiments particularly rely upon the level of an ANTXR1 expression and the level of expression of other biomarkers in a cancerous tissue from the subject. Also provided herein are methods for determining the same.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20%, ±10%, ±5%, ±1%, or ±0.1% from the specified value, as such variations are appropriate to perform the disclosed methods.

The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, the terms “comprising,” “including,” “containing” and “characterized by” are exchangeable, inclusive, open-ended and do not exclude additional, unrecited elements or method steps. Any recitation herein of the term “comprising,” particularly in a description of components of a composition or in a description of elements of a device, is understood to encompass those compositions and methods consisting essentially of and consisting of the recited components or elements.

As used herein, the term “consisting of” excludes any element, step, or ingredient not specified in the claim element.

As used herein the term “Seneca Valley Virus” or “SVV” encompasses wild type SVV or an SVV derivative. Exemplary suitable SVV strains include SVV-001 (SEQ ID NO: 1 and SEQ ID NO: 2, see Table 24 below), NTX-010, and the SVV strain having ATCC Patent Deposit Number PTA-5343. As used herein, the term “derivative” specifies that a derivative of a virus can have a nucleic acid or amino acid sequence difference in respect to a template viral nucleic acid or amino acid sequence. For instance, an SVV derivative can refer to an SVV that has a nucleic acid or amino acid sequence different with respect to the wild-type SVV nucleic acid or amino acid sequence of ATCC Patent Deposit Number PTA-5343. In some embodiments, the SVV derivative encompasses an SVV mutant, an SVV variant or a modified SVV (e.g. genetically engineered SVV). In one embodiment, the SVV derivative is a SVV virus modified to express a therapeutic protein. Examples of such SVV derivatives may be found in Intl. Publ. No. WO2022/159508, published on Jul. 28, 2022, the disclosure of which is incorporated herein by reference in its entirety. Exemplary suitable SVV derivatives are the following SVV armed constructs: (1) SVV-001/Enhanced IL-2 (SVV-024); (2) SVV-001-Anti-PD-L1 (SVV-012); (3) SVV-001-CXCL9 (SVV-037); (4) SVV-001/TGF beta decoy (SVV-044); (5) SVV-001/Nitroreductase (SVV-058); (6) SVV-001/IL2-IL15 Fusion Protein (SVV-069); and (7) SVV-001/Ovalbumin epitope (SVV-077). These armed SVV constructs were designed by Seneca Therapeutics, Inc. In other embodiments, the SVV derivative may be ONCR-788. In some embodiments, the SVV derivative is modified to be capable of recognizing different cell receptors or to be capable of evading the immune system while still being able to invade, replicate and kill the cell of interest (i.e. cancer cell). In other embodiments, SVV is modified to express an agent that is useful for treating cancer. In general, an SVV can be derived from a pre-existing stock of virus that is passaged to produce more viruses. SVV can also be derived from a plasmid.

As used herein, “higher” refers to expression levels which are at least 10% or more, for example, 20%, 30%, 40%, or 50%, 60%, 70%, 80%, 90% higher or more, and/or 1.1-fold, 1.2-fold, 1.4-fold, 1.6-fold, 1.8-fold, 2.0-fold higher or more, and any and all whole or partial increments therebetween, than a control reference. As disclosed herein an expression level higher than a reference value refers to an expression level (mRNA or protein) that is higher than a normal or control expression level from an expression (mRNA or protein) measured in a healthy subject or defined or used in the art. The control expression level may be the level detected in cancers that are resistant to treatment with SVV.

As used herein, “lower” refers to expression levels which are at least 10% lower or more, for example, 20%, 30%, 40%, or 50%, 60%, 70%, 80%, 90% lower or more, and/or 1.1-fold, 1.2-fold, 1.4-fold, 1.6-fold, 1.8-fold, 2.0-fold lower or more, and any and all whole or partial increments in between, than a control reference. As disclosed herein an expression level lower than a reference value refers to an expression level (mRNA or protein) that is lower than a normal or control expression level from an expression (mRNA or protein) measured in a healthy subject or defined or used in the art. The control expression level may be the level detected in cancers that are resistant to treatment with SVV.

As used herein, the terms “control” and “reference” can be used interchangeably to refer to a value that is used as a standard of comparison.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime. For purposes herein, a combination therapy can include a treatment regime that includes administration of an oncolytic virus and another anti-cancer agent, each for treating the same hyperproliferative disease or conditions, such as the same tumor or cancer. In preferred embodiments, the combination therapy includes administration of SVV in conjunction with one or more checkpoint inhibitor in a patient before, during, or after delivery of SVV.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, plaque forming units (PFU) refers to a measure of number of infectious virus particles. It is determined by plaque forming assay.

As used herein, viral genomes (VG) refers to a measure of the number of viral genomes as determined by polymerase chain reaction.

As used herein, multiplicity of infection (MOI) refers to the average number of virus particles infecting each cell. MOI can be related to PFU by the following formula: Multiplicity of infection (MOI)=Plaque forming units (PFU) of virus used for infection/number of cells.

The term “RNA” as used herein is defined as ribonucleic acid.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually develop a disease condition. Thus, for example, the term treatment includes the administration of an agent prior to or following the onset of a disease or disorder thereby preventing or removing one or more signs of the disease or disorder. As another example, administration of the agent after clinical manifestation of the disease to combat the symptoms of the disease comprises “treatment” of the disease.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to intra-tumoral, intravenous, intrapleural, oral, aerosol, parenteral, ophthalmic, pulmonary, and topical administration.

The language “pharmaceutically acceptable carrier” includes a pharmaceutically acceptable salt, pharmaceutically acceptable material, composition, or carrier, such as a liquid or solid filler, diluent, excipient, solvent, or encapsulating material, involved in carrying or transporting a compound(s) (e.g. SVV and/or a checkpoint inhibitor) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound and are physiologically acceptable to the subject. Supplementary active compounds may also be incorporated into the compositions.

As used herein, the term “effective amount” or “therapeutically effective amount” means the amount of the virus genome, virus particle, or infectious units generated from a vector, which is required to prevent the particular disease condition, or which reduces the severity of and/or ameliorates the disease condition or at least one symptom thereof or condition associated therewith.

A “subject” or “patient,” as used herein, may be a human or non-human mammal. Non-human mammals include, for example, livestock and pets, such as ovine, bovine, porcine, canine, feline and murine mammals. Preferably, the subject is a human.

As used herein, the terms “cold tumor” refers to a tumor that is resistant to the antitumor immune response of the subject having the tumor (“cold tumor”). As used herein, the terms “hot tumor” refers to a tumor that is susceptible to the antitumor immune response of the subject having the tumor (“hot tumor”). For example, cold tumors refer to cancers that contain a low number of infiltrating T cells and are not recognized and do not provoke a strong response by the immune system, making them difficult to treat with current immunotherapies. Cancers that are classically immunologically cold include but are not limited to glioblastomas as well as ovarian, prostate, pancreatic, and most breast cancers. In contrast, immunologically hot tumors contain high levels of infiltrating T cells and more antigens, making them more recognizable by the immune system and more likely to trigger a strong immune response. Non limiting examples of cancers considered to be immunologically hot are bladder, head and neck, kidney, melanoma, and non-small cell lung cancers.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, inclusive of each of the range endpoints, for example, 1, 2, 2.7, 3, 4, 5, 5.3, and 6. This applies regardless of the breadth of the range.

Biomarkers to Identify Cancers Susceptible to Treatment with SVV

The disclosure is directed to one or more biomarkers that can be used to assess whether a cancer is susceptible to treatment with SVV. Furthermore, the disclosure is directed to one or more biomarkers that can be used to assess whether a patient suffering from cancer should not be treated with SVV.

In particular, in certain embodiments, presence of ANTXR1 and one or more of the biomarkers or biomarker profiles described herein is indicative that the cancer is susceptible to treatment with SVV. In other embodiments, the presence of the one or more of biomarkers or biomarker profiles described herein is indicative that the cancer is susceptible to treatment with SVV (i.e. SVV-S (sensitive to treatment with SVV)).

In one embodiment, the one or more biomarkers may be selected from the following: ELN; CD248; COL25A1; ALPL; SEMA5B; ATP1B2; NEUROD1; VATIL; MFAP4; LPPR4; HPCA; ISLR; NHLH1; CUX2; CAMKV; ACCN4; JPH4; HIF3A; CNTFR; and NTN3. In certain embodiments, two or more, alternatively three or more, alternatively four or more, alternatively five or more, alternatively six more, alternatively seven or more, alternatively eight or more, alternatively nine or more, alternatively ten or more, alternatively eleven or more, alternatively twelve or more, alternatively thirteen or more, alternatively fourteen or more, alternatively fifteen or more, alternatively sixteen or more, alternatively seventeen or more, alternatively eighteen or more, alternatively nineteen or more, alternatively each of these biomarkers may be used in the assays and methods of the disclosure. In certain embodiments, ANTXR1 is also included as a suitable biomarker.

In one embodiment, the one or more biomarkers may be selected from the following: ELN; CD248; COL25A1; ALPL; SEMA5B; ATP1B2; NEUROD1; VATIL; MFAP4; LPPR4; HPCA; ISLR; NHLH1; CUX2; CAMKV; ACCN4; JPH4; HIF3A; CNTFR; EFS; NEFH; SOX11; and NTN3. In certain embodiments, two or more, alternatively three or more, alternatively four or more, alternatively five or more, alternatively six more, alternatively seven or more, alternatively eight or more, alternatively nine or more, alternatively ten or more, alternatively eleven or more, alternatively twelve or more, alternatively thirteen or more, alternatively fourteen or more, alternatively fifteen or more, alternatively sixteen or more, alternatively seventeen or more, alternatively eighteen or more, alternatively nineteen or more, alternatively twenty or more, alternatively twenty one or more, alternatively twenty two or more, alternatively each of these biomarkers may be used in the assays and methods of the disclosure. In certain embodiments, ANTXR1 is also included as a suitable biomarker.

In another embodiment, the one or more biomarkers may be selected from the following: ELN; CD248; EFS; ALPL; SEMA5B; ATP1B2; NEUROD1; VATIL; MFAP4; NEFH; HPCA; ISLR; NHLH1; SOX11; CAMKV; ACCN4; JPH4; HIF3A; CNTFR; and NTN3. In certain embodiments, two or more, alternatively three or more, alternatively four or more, alternatively five or more, alternatively six more, alternatively seven or more, alternatively eight or more, alternatively nine or more, alternatively ten or more, alternatively eleven or more, alternatively twelve or more, alternatively thirteen or more, alternatively fourteen or more, alternatively fifteen or more, alternatively sixteen or more, alternatively seventeen or more, alternatively eighteen or more, alternatively nineteen or more, alternatively each of these biomarkers may be used in the assays and methods of the disclosure. In certain embodiments, ANTXR1 is also included as a suitable biomarker.

In yet another embodiment, the one or more biomarkers may be each of ELN, CD248, EFS, ALPL, and SEMA5B. In yet another embodiment, the one or more biomarkers may be each of ELN, CD248, EFS, ALPL, SEMA5B, ATPIB2, NEUROD1, VATIL, MFAP4, and NEFH. In yet another embodiment, the one or more biomarkers may be each of: COL25A1, CSF2RA, NCAMI, NDN, ST8SIA2, and TSPYL5. In certain embodiments, ANTXR1 is also included as a suitable biomarker.

In yet another embodiment, presence (expression) or increased expression of each of the following biomarkers is indicative that the cancer is susceptible to treatment with SVV: EFS, MFAP4, NTN3, SOX11, NEFH, CNTFR, JPH4, and ANTXR1.

Exemplary biomarker profiles that may be used to identify if a cancer is SVV-S are shown in the Table 1 below. In each of these biomarker profiles, a cancer that is SVV-S may be identified based on the expression of one or more of the biomarkers forming the profile to identify SVV-S cancers. Alternatively, in each of these biomarker profiles, a cancer that is SVV-S may be identified based on the expression of all of the biomarkers forming the profile to identify SVV-S cancers.

TABLE 1
Suitable Exemplary Biomarker Profiles for identifying SVV-S cancer.
The biomarkers for the 8 gene assay are shown as Profile 7.

| Biomarker Profile | One or more biomarkers forming profile to identify SVV-S cancers |
|---|---|
| Profile 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| Profile 2 | ELN, CD248, EFS, ALPL, SEMA5B, ATP1B2, NEUROD1, |
| | VAT1L, MFAP4, NEFH, HPCA, ISLR, NHLH1, SOX11, |
| | CAMKV, ACCN4, JPH4, HIF3A, CNTFR, and NTN3 |
| Profile 3 | ELN, CD248, EFS, ALPL, and SEMA5B |
| Profile 4 | ELN, CD248, EFS, ALPL, SEMA5B, ATP1B2, NEUROD1, |
| | VAT1L, MFAP4, and NEFH |
| Profile 5 | COL25A1, CSF2RA, NCAM1, NDN, ST8SIA2, and TSPYL5 |
| Profile 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| Profile 7 | EFS, MFAP4, NTN3, SOX11, NEFH, CNTFR, JPH4, and |
| | ANTXR1 |
| Profile 8 | EFS, MFAP4 NTN3, SOX11, NEFH, and CNTFR |
| Profile 9 | ELN, CD248, ALPL, SEMA5B, ATP1B2, NEUROD1, VAT1L, |
| | MFAP4, HPCA, ISLR, NHLH1, CAMKV, ACCN4, and ANTXR1 |
| Profile 10 | ELN, CD248, EFS, ALPL, SEMA5B, ATP1B2, NEUROD1, |
| | VAT1L, MFAP4, NEFH, HPCA, ISLR, NHLH1, SOX11, |
| | CAMKV, and ANTXR1 |
| Profile 11 | SOX11, EFS, NEFH, MFAP4, CNTFR, and ANTXR1 |
| Profile 12 | ELN, CD248, EFS, ALPL, SEMA5B, ATP1B2, NEUROD1, |
| | VAT1L, MFAP4, NEFH, HPCA, ISLR, NHLH1, SOX11 |
| Profile 13 | ELN, CD248, ALPL, SEMA5B, ATP1B2, NEUROD1, VAT1L, |
| | MFAP4, HPCA, ISLR, NHLH1 |
| Profile 14 | SOX11, EFS, NEFH, MFAP4, CNTFR |
| Profile 15 | ELN, CD248, COL25A1, ALPL, SEMA5B, ATP1B2, NEUROD1, |
| | VAT1L, MFAP4 |

Additionally, any of the biomarker profiles described in the examples below and the accompanying figures may be used.

Detection of the at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, or at least 20 biomarkers of the biomarker profile may identify an SVV-sensitive tumor with a sensitivity of at least about 60%.

Detection of the at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, or at least 20 biomarkers of the biomarker profile may identify an SVV-sensitive tumor with a specificity of at least about 60%.

Detection of the at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, or at least 20 biomarkers of the biomarker profile may identify an SVV-sensitive tumor with a positive predictive value of at least about 60%.

Detection of the at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, or at least 20 biomarkers of the biomarker profile may identify an SVV-sensitive tumor with a negative predictive value of at least about 60%.

Detection of the at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, or at least 20 biomarkers of the biomarker profile may identify an SVV-sensitive tumor with a sensitivity of at least about 60%, a specificity of at least about 60%, a positive predictive value of at least about 60%, and/or a negative predictive value of at least about 60%.

The sensitivity of at least about 60% may be a sensitivity of at least about 60%, at least about 65%, at least about 70%, at least about 75%, at least about 80%, at least about 85%, at least about 90%, or at least about 95%.

The specificity of at least about 60% may be a specificity of at least about 60%, at least about 65%, at least about 70%, at least about 75%, at least about 80%, at least about 85%, at least about 90%, or at least about 95%.

The positive predictive value of at least about 60% may be a positive predictive value of at least about 60%, at least about 65%, at least about 70%, at least about 75%, at least about 80%, at least about 85%, at least about 90%, or at least about 95%.

The negative predictive value of at least about 60% may be a negative predictive value of at least about 60%, at least about 65%, at least about 70%, at least about 75%, at least about 80%, at least about 85%, at least about 90%, or at least about 95%.

The probability for identifying an SVV-S cancer with the sensitivity of at least about 60%, the specificity of at least about 60%, the positive predictive value of at least about 60%, and/or the negative predictive value of at least about 60% may be about 50%, about 60%, about 70%, about 80%, about 90%, about 95%, or about 99%. The probability for identifying an SVV-S cancer with the sensitivity of at least about 60%, the specificity of at least about 60%, the positive predictive value of at least about 60%, and/or the negative predictive value of at least about 60% may be at least about 50%, at least about 55%, at least about 60%, at least about 65%, at least about 70%, at least about 75%, at least about 80%, at least about 85%, at least about 90%, at least about 95%, or at least about 99%.

Detection of the at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, or at least 20 biomarkers of the biomarker profile may identify an SVV-sensitive tumor with a sensitivity between about 60% and 95%, a specificity between about 60% and 95%, a positive predictive value between about 60% and 95%, and/or a negative predictive value between about 60% and 95% with the probability of between about between about 50% and 99%.

Detection of the at least one biomarker, at least two biomarkers, at least three biomarkers, at least four biomarkers, at least five biomarkers, at least six biomarkers, at least seven biomarkers, at least eight biomarkers, at least nine biomarkers, at least ten biomarkers, at least 11 biomarkers, at least 12 biomarkers, at least 13 biomarkers, at least 14 biomarkers, at least 15 biomarkers, at least 16 biomarkers, at least 17 biomarkers, at least 18 biomarkers, at least 19 biomarkers, or at least 20 biomarkers of the biomarker profile may identify an SVV-sensitive tumor with a sensitivity between about 80% and 95%, a specificity between about 70% and 95%, a positive predictive value between about 80% and 95%, and/or a negative predictive value between about 80% and 95% with the probability of between about between about 70% and 99%. For example, detection of expression of the genes within the 8-gene panel can identify an SVV-sensitive tumor with a sensitivity of about 80%, about 82%, about 85%, about 87%, about 90%, about 92%, or about 95%; specificity of about 70%, about 72%, about 74%, about 77%, about 80%, about 82%, about 85%, about 87%, about 90%, about 92%, or about 95%; positive predictive value of about 80%, about 82%, about 85%, about 88%, about 90%, about 92%, or about 95%; and/or negative predictive value of about 80%, about 81%, about 85%, about 87%, about 90%, about 92%, or about 95%; with prediction accuracy of about 80%, about 81%, about 86%, about 87%, or about 90%.

For example, detection of expression of the genes within the 8-gene panel can identify an SVV-sensitive tumor with a sensitivity of about 92%, specificity of about 74%, positive predictive value of about 88%, and/or negative predictive value of about 81% with prediction accuracy of about 86%.

In certain embodiments, the presence of the biomarker may be determined based on the expression of the marker. In other embodiments, the presence of the biomarkers may be based on increased expression of the marker.

In certain embodiments, the biomarkers (genes) are all up-regulated in SVV-S cells and tend to have very low expression in SVV-R cells.

In certain embodiments, increased expression level of a biomarker refers to a statistically significant higher expression in the SVV-S cells when compared to that in the SVV-R cells. p values less than 0.05 are considered statistically significant. The statistically significant increase can be based on variables including the number of subjects tested or a specific method of detection or measurement. In some embodiments, in circumstances in which multiple biomarkers are detected, p values less than 0.05 for the combination of the biomarkers can be considered statistically significant. The increased expression can be as compared to the expression of a control group.

Accordingly in certain embodiments, the invention is directed to in vitro methods of identifying a cancer that is susceptible based on detecting the presence of the one or more biomarkers described herein. The methods also include the step of obtaining or providing a cancer sample. The cancer sample may be obtained by conventional means. In one embodiment, the cancer sample is obtained using a biopsy. In another embodiment, the methods may rely on FFPE tumor blocks allowing for patient screening with previous biopsies.

The assay methods of the disclosure enable identification of cancer patients that will be susceptible to treatment with SVV. The disclosure also relates to methods of treating cancer that has been identified as being susceptible to treatment with SVV by administering SVV regardless of the contemplated administration route. In certain embodiments, the patient may be treated by intra-tumoral administration of SVV. In other embodiments, the patient may be treated by intravenous administration of SVV.

The treatment of cancer provided herein may include the treatment of solid tumors or the treatment of metastasis. Metastasis is a form of cancer wherein the transformed or malignant cells are traveling and spreading the cancer from one site to another. Such cancers include cancers of the skin, breast, brain, cervix, testes, etc. More particularly, cancers may include, but are not limited to the following organs or systems: cardiac, lung, gastrointestinal, genitourinary tract, liver, bone, nervous system, gynecological, hematologic, skin, and adrenal glands. More particularly, the methods herein can be used for treating gliomas (Schwannoma, glioblastoma, astrocytoma), neuroblastoma, neuroendocrine tumor, neuroendocrine carcinoma (e.g., large cell neuroendocrine carcinoma, extra thoracic small cell carcinoma, poorly differentiated neuroendocrine carcinoma), pheochromocytoma, paraganglioma, meningioma, adrenocortical carcinoma, kidney cancer, vascular cancer of various types, osteoblastic osteocarcinoma, prostate cancer, ovarian cancer, uterine leiomyomas, salivary gland cancer, choroid plexus carcinoma, mammary cancer, pancreatic cancer, colon cancer, and megakaryoblastic leukemia. Skin cancer includes malignant melanoma, basal cell carcinoma, squamous cell carcinoma, Kaposi's sarcoma, moles dysplastic nevi, lipoma, angioma, dermatofibroma, keloids, rhabdomyosarcoma, medulloblastoma, and psoriasis.

In some embodiments, the cancer treated by the presently disclosed methods comprises a triple negative breast cancer, a small cell lung cancer, a non-small cell lung cancer, a non-small cell squamous carcinoma, an adenocarcinoma, a glioblastoma, a skin cancer, a hepatocellular carcinoma, a colon cancer, a cervical cancer, an ovarian cancer, an endometrial cancer, a neuroendocrine cancer, a pancreatic cancer, a thyroid cancer, a kidney cancer, a bone cancer, an esophagus cancer, or a soft tissue cancer. In other embodiments, the cancer is a neuroblastoma or a melanoma. In yet another embodiment, the cancer is a neuroendocrine cancer (e.g., neuroendocrine tumor or a neuroendocrine carcinoma selected from large cell neuroendocrine carcinoma, extra thoracic small cell carcinoma, poorly differentiated neuroendocrine carcinoma) or a small cell lung cancer (SCLC) tumor.

### Reference Value or Control

The methods provided herein include comparing a measured expression level of ANTXR1 and/or the one or more biomarkers in a cancer sample from a subject to a reference value of expression (i.e. the control expression level) of ANTXR1 or the one or more biomarkers.

In one embodiment, the reference level of expression of ANTXR1 may be obtained by measuring the expression level of ANTXR1 in a healthy subject. Preferably, the healthy subject is a subject of similar age, gender and race and has never been diagnosed with any type of sever disease particularly any type of cancer.

In another embodiment, the reference value of expression of ANTXR1 and/or the one or more biomarkers may be obtained by measuring the expression level of ANTXR1 and/or the one or more biomarkers in a cancer that is SVV-R.

In another embodiment, the reference value of expression of ANTXR1 is a value for expression of ANTXR1 that is accepted in the art. This reference value can be baseline value calculated for a group of subjects based on the average or mean values of ANTXR1 expression by applying standard statistically methods.

In one embodiment, the reference level of expression of the one or more biomarkers may be obtained by measuring the expression level of the one or more biomarkers in a healthy subject. Preferably, the healthy subject is a subject of similar age, gender and race and has never been diagnosed with any type of sever disease particularly any type of cancer.

In another embodiment, the reference value of expression of the one or more biomarkers may be obtained by measuring the expression level of the one or more biomarkers in a cancer that is SVV-R.

In one embodiment, the expression level is determined by a method selected from the group consisting of detecting mRNA of the gene, detecting a protein encoded by the gene, and detecting a biological activity of the protein encoded by the gene.

In certain aspects of the present invention, the expression level of ANTXR1 and/or the one or more biomarkers is determined in a cancerous sample from a subject. The sample preferably includes tumor cells, any fluid from the surrounding of tumor cells (e.g. leukemic blood, or tumor tissue) or any fluid that is in physiological contact or proximity with the tumor, or any other body fluid in addition to those recited herein should also be considered to be included herein.

### Methods of Measurement

Any in vitro method known to those in the art can be employed for determining the expression level of ANTXR1, and the one or more biomarkers at the transcriptional or translational level. For example, a microarray can be used. Microarrays are known in the art and consist of a surface to which probes that correspond in sequence to gene products (e.g. mRNAs, cDNA, polypeptides, fragments thereof etc.) can be specifically hybridized or bound to a known position. To detect at least one gene of interest, a hybridization sample is formed by contacting the test sample with at least one nucleic acid probe. A preferred probe for detecting ANTXR1 and/or one or more biomarkers is a labeled nucleic acid probe capable of hybridizing to ANTXR1 and/or the one or more biomarkers. The nucleic acid probe can be, for example, a full-length nucleic acid molecule, or a portion thereof, such as an oligonucleotide of at least 10, 15, or 20 nucleotides in length and sufficient to specifically hybridize under stringent conditions to the appropriate target. The hybridization sample is maintained under conditions which are sufficient to allow specific hybridization of the nucleic acid probe to a target of interest. Specific hybridization can be performed under high stringency conditions or moderate stringency conditions, as appropriate. In a preferred embodiment, the hybridization conditions for specific hybridization are high stringency. Specific hybridization, if present, is then detected using standard methods. If specific hybridization occurs between the nucleic acid probe and a gene in the test sample, the sequence that is present in the nucleic acid probe is also present in the mRNA of the subject. More than one nucleic acid probe can also be used. Hybridization intensity data detected by the scanner are automatically acquired and processed by the Affymetrix® Microarray Suite (MASS) software. Raw data is normalized to expression levels using a target intensity of 150. An alternate method to measure mRNA expression profiles of a small number of different genes is by e.g. either classical TaqMan® Gene Expression Assays or TaqMan® Low Density Array—micro fluidic cards (Applied Biosystems). In some embodiment, this disclosure preferably utilizes a qPCR system. Non-limiting examples include commercial kits such as the PrimePCRPathways® commercially available from Bio-Rad® (Berkley, California).

The transcriptional state of a sample, particularly mRNAs, may also be measured by other nucleic acid expression technologies known in the art. mRNA can be isolated from the sample using any method known to those in the art. Non-limiting examples include commercial kits, such as the RNeasy® commercially available from Qiagen (Netherlands) or the Mini Kit the TRI Reagent® commercially available from Molecular Research Center, Inc. (Cincinnati, Ohio), can be used to isolate RNA. Generally, the isolated mRNA may be amplified using methods known in the art. Amplification systems utilizing, for example, PCR or RT-PCR methodologies are known to those skilled in the art. For a general overview of amplification technology, see, for example, Dieffenbach et al., PCR Primer: A Laboratory Manual, Cold Spring Harbor Laboratory Press, New York (1995).

Another accurate method for profiling mRNA expression can the use of Next Generation Sequencing (NGS) including first, second, third as well as subsequent Next Generations Sequencing technologies.

In other embodiments, the transcriptional state of a sample, particularly mRNAs, may also be measured by RNA sequencing (RNAseq), or by using nCounter® platform (NanoString Technologies, Inc., Seattle, WA).

In other aspects provided herein, determining the amount, or detecting the biological activity of a peptide, polypeptide can be achieved by all known means in the art for determining the amount of a peptide or polypeptide in a sample. These means comprise immunoassay devices and methods which may utilize labeled molecules in various sandwich, competition, or other assay formats. Such assays will develop a signal which is indicative for the presence or absence of the peptide or polypeptide. Moreover, the signal strength can, preferably, be correlated directly or indirectly (e.g. reverse-proportional) to the amount of polypeptide present in a sample. Further suitable methods comprise measuring a physical or chemical property specific for the peptide or polypeptide such as its precise molecular mass or NMR spectrum. These methods comprise, preferably, biosensors, optical devices coupled to immunoassays, biochips, analytical devices such as mass-spectrometers, NMR-analyzers, HPLC, FPLC, or chromatography devices. Further, methods include, Western blots, micro-plate ELISA-based methods, fully-automated or robotic immunoassays (available for example on Elecsys™ analyzers), CBA (an enzymatic Cobalt Binding Assay, available for example on Roche-Hitachi™ analyzers), and latex agglutination assays (available for example on Roche-Hitachi™ analyzers).

In some embodiments, for the various methods disclosed herein, the expression level of ANTXR1 is determined based on the level of an ANTXR1 mRNA or an ANTXR1 protein and the expression level of one or more biomarkers is determined based on the level of the biomarker mRNA or the biomarker protein.

### Combination Therapies

The compositions and methods for treating a cancer in a subject using SVV described herein, which rely on the testing of one or more biomarkers, may be useful when combined with at least one additional compound useful for treating cancer. The additional compound may comprise a commercially available compound known to treat, prevent, or reduce the symptoms of cancer and/or metastasis.

In one aspect, the pharmaceutical composition disclosed herein may be used in combination with a therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein may be used in combination with a radiation therapy.

Most alkylating agents are cell cycle non-specific. In specific aspects, they stop tumor growth by cross-linking guanine bases in DNA double-helix strands. Non-limiting examples include busulfan, carboplatin, chlorambucil, cisplatin, cyclophosphamide, dacarbazine, ifosfamide, mechlorethamine hydrochloride, melphalan, procarbazine, thiotepa, and uracil mustard.

Anti-metabolites prevent incorporation of bases into DNA during the synthesis(S) phase of the cell cycle, prohibiting normal development and division. Non-limiting examples of antimetabolites include drugs such as 5-fluorouracil, 6-mercaptopurine, capecitabine, cytosine arabinoside, floxuridine, fludarabine, gemcitabine, methotrexate, and thioguanine.

Antitumor antibiotics generally prevent cell division by interfering with enzymes needed for cell division or by altering the membranes that surround cells. Included in this class are the anthracyclines, such as doxorubicin, which act to prevent cell division by disrupting the structure of the DNA and terminate its function. These agents are cell cycle non-specific. Non-limiting examples of antitumor antibiotics include aclacinomycin, actinomycin, anthramycin, azaserine, bleomycins, cactinomycin, calicheamicin, carubicin, caminomycin, carzinophilin, chromomycin, dactinomycin, daunorubicin, detorubicin, 6-diazo-5-oxo-L-norleucine, doxorubicin, epirubicin, esorubicin, idarubicin, marcellomycin, mitomycins, mitoxantrone, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin.

Plant alkaloids inhibit or stop mitosis or inhibit enzymes that prevent cells from making proteins needed for cell growth. Frequently used plant alkaloids include vinblastine, vincristine, vindesine, and vinorelbine. However, the invention should not be construed as being limited solely to these plant alkaloids.

The taxanes affect cell structures called microtubules that are important in cellular functions. In normal cell growth, microtubules are formed when a cell starts dividing, but once the cell stops dividing, the microtubules are disassembled or destroyed. Taxanes prohibit the microtubules from breaking down such that the cancer cells become so clogged with microtubules that they cannot grow and divide. Non-limiting exemplary taxanes include paclitaxel and docetaxel.

Hormonal agents and hormone-like drugs are utilized for certain types of cancer, including, for example, leukemia, lymphoma, and multiple myeloma. They are often employed with other types of chemotherapy drugs to enhance their effectiveness. Sex hormones are used to alter the action or production of female or male hormones and are used to slow the growth of breast, prostate, and endometrial cancers. Inhibiting the production (aromatase inhibitors) or action (tamoxifen) of these hormones can often be used as an adjunct to therapy. Some other tumors are also hormone dependent. Tamoxifen is a non-limiting example of a hormonal agent that interferes with the activity of estrogen, which promotes the growth of breast cancer cells.

Miscellaneous agents include chemotherapeutics such as bleomycin, hydroxyurea, L-asparaginase, and procarbazine.

Other examples of chemotherapeutic agents include, but are not limited to, the following and their pharmaceutically acceptable salts, acids and derivatives: MEK inhibitors, such as but not limited to, refametinib, selumetinib, trametinib or cobimetinib; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, ranimustine; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatrexate; defofamine; demecolcine; diaziquone; eflornithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K (PSK); razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel (TAXOLO, Bristol-Myers Squibb Oncology, Princeton, N.J.) and docetaxel (TAXOTERE, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; and capecitabine.

An anti-cell proliferation agent can further be defined as an apoptosis-inducing agent or a cytotoxic agent. The apoptosis-inducing agent may be a granzyme, a Bcl-2 family member, cytochrome C, a caspase, or a combination thereof. Exemplary granzymes include granzyme A, granzyme B, granzyme C, granzyme D, granzyme E, granzyme F, granzyme G, granzyme H, granzyme I, granzyme J, granzyme K, granzyme L, granzyme M, granzyme N, or a combination thereof. In other specific aspects, the Bcl-2 family member is, for example, Bax, Bak, Bcl-Xs, Bad, Bid, Bik, Hrk, Bok, or a combination thereof.

In additional aspects, the caspase is caspase-1, caspase-2, caspase-3, caspase-4, caspase-5, caspase-6, caspase-7, caspase-8, caspase-9, caspase-10, caspase-11, caspase-12, caspase-13, caspase-14, or a combination thereof. In specific aspects, the cytotoxic agent is TNF-α, gelonin, Prodigiosin, a ribosome-inhibiting protein (RIP), Pseudomonas exotoxin, Clostridium difficile Toxin B, Helicobacter pylori VacA, Yersinia enterocolitica YopT, Violacein, diethylenetriaminepentaacetic acid, irofulven, Diptheria Toxin, mitogillin, ricin, botulinum toxin, cholera toxin, saporin 6, or a combination thereof.

An immunotherapeutic agent may be, but is not limited to, an interleukin-2 or other cytokine, an inhibitor of programmed cell death protein 1 (PD-1) signaling such as a monoclonal antibody that binds to cytotoxic T lymphocytes associated antigen A-4 (CTLA4) or PD-1, e.g., ipilimumab or nivolumab. The immunotherapeutic agent can also block CTLA-4 signaling and it can also relate to cancer vaccines and dendritic cell-based therapies. In one embodiment, the inhibitor of PD-1 signaling is nivolumab. In one embodiment, the inhibitor of CTLA-4 signaling is ipilimumab. The immunotherapeutic agent may be, but is not limited to nivolumab, ipilimumab, or nivolumab and ipilimumab combination.

In one embodiment the subject suffering from cancer is administered at least one anti-cancer therapeutic agent selected from the group consisting of: a checkpoint inhibitor, a PD-1 inhibitor, a programmed cell death ligand-1 (PD-L1) inhibitor, a CTLA-4 inhibitor, a cytokine, a growth factor, a photosensitizing agent, a toxin, a siRNA molecule, a signaling modulator, an anti-cancer antibiotic, an anti-cancer antibody, an angiogenesis inhibitor, a chemotherapeutic compound, anti-metastatic compound, an immunotherapeutic compound, a chimeric antigen receptor (CAR) therapy, a dendritic cell-based therapy, and a combination of any thereof. In one embodiment, the at least one anti-cancer therapeutic agent is administered formerly, simultaneously, or subsequently to the administering of the SVV.

Combination Therapy with SVV and an Immune Checkpoint Inhibitor

An exemplary method of combination therapy for treating cancer in a subject is a method of administering an effective amount of a treatment comprising SVV and an immune checkpoint inhibitor to the subject. The method may be for treating a cancer that expresses one or more of the biomarkers selected from one of the biomarker profiles presented in Table 1. The method may comprise administering the treatment during a treatment period lasting until cancer progression, end of treatment, or unacceptable toxicity. In some embodiments, the cancer expresses EFS, MFAP4, NTN3, SOX11, NEFH, CNTFR, and JPH4 at at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold greater expression relative to a housekeeping gene, and expresses ANTXR1. The expression of the one or more biomarkers may be increased relative to the expression of the one or more biomarkers in cancers that are resistant to treatment with SVV.

The method may further comprise detecting the expression of the one or more biomarkers comprising EFS, MFAP4, NTN3, SOX11, NEFH, CNTFR, JPH4, and ANTXR1.

The method may comprise administering the treatment comprising an effective amount of SVV and an effective amount of an immune checkpoint inhibitor to the subject throughout the treatment period. The method may comprise administering an effective amount of an immune checkpoint inhibitor to the subject after the treatment period. In some embodiments, the immune checkpoint inhibitor is a programmed death receptor-1 (PD-1) inhibitor. In some embodiments, the immune checkpoint inhibitor is nivolumab.

In some embodiments of the method, SVV is encoded by SEQ ID NO: 2. In some embodiments of the method, SVV is encoded by SEQ ID NO: 1.

The method may comprise administering an effective amount of SVV intratumorally and administering an effective amount of the immune checkpoint inhibitor intravenously. The method may comprise administering the immune checkpoint inhibitor intravenously after administering SVV intratumorally.

In some embodiments, the immune checkpoint inhibitor is administered every 14 days starting 14 days after initial administration of the SVV. The SVV may be administered once during a treatment period lasting between about four weeks and 24 weeks. The SVV may be administered twice a week, once a week, once every two weeks, once every three weeks, or once every four weeks during the treatment period lasting between about four weeks and 24 weeks.

The SVV may be administered at a total dose between about 2×10³ viral genomes and 5×10¹⁰ viral genomes per subject per day. For example, the SVV may be administered intratumorally once, twice, three times, four times, five times, six times, seven times, eight times, nine times, ten times, 11 times, 12 times, 13 times, 14 times, 15 times, 16 times, 17 times, 18 times, 19 times, 20 times, 21 times, 22 times, 23 times, or 24 times during the treatment period lasting between about four weeks and 24 weeks. The SVV may be administered intratumorally at a total dose between about 2×10³ viral genomes (VG) and about 5×10¹⁰ VG per subject per day. For example, the SVV may be administered at a total dose between about 2×10³ VG and about 5×10¹⁰ VG, between about 2×10³ VG and about 4×10¹⁰ VG, between about 2×10³ VG and about 3×10¹⁰ VG, between about 2×10³ VG and about 2×10¹⁰ VG, or between about 2×10⁵ VG and about 2×10¹⁰ VG per subject per day. The SVV may be administered at a total dose of about 2×10³ VG, about 2×10⁴ VG, about 2×10⁵ VG, about 2×10⁶ VG, about 2×10⁷ VG, about 2×10⁸ VG, about 2×10⁹ VG, about 2×10¹⁰ VG, about 3×10³ VG, about 3×10⁴ VG, about 3×10⁵ VG, about 3×10⁶ VG, about 3×10⁷ VG, about 3×10⁸ VG, about 3×10⁹ VG, about 3×10¹⁰ VG, about 4×10³ VG, about 4×10⁴ VG, about 4×10⁵ VG, about 4×10⁶ VG, about 4×10⁷ VG, about 4×10⁸ VG, about 4×10⁹ VG, about 4×10¹⁰ VG, about 5×10³ VG, about 5×10⁴ VG, about 5×10⁵ VG, about 5×10⁶ VG, about 5×10⁷ VG, about 5×10⁸ VG, about 5×10⁹ VG, or about 5×10¹⁰ VG, per subject per day.

In some embodiments, the SVV is administered at a total dose between about 2.2×10⁸ VG and 2.2×10¹⁰ VG per subject per day, such as about 2.2×10⁸ VG, about 2.2×10⁹ VG, or about 2.2×10¹⁰ VG per subject per day. In the context of intratumoral SVV administration, the term “at a total dose . . . per subject per day” refers to the total dose the subject will receive that day, not the dose per lesion.

The immune checkpoint inhibitor may be administered intravenously (IV) once every 14 days at a treatment dose during the treatment period. The immune checkpoint inhibitor may be administered once every four weeks at a maintenance dose following the treatment period. The immune checkpoint inhibitor may be nivolumab administered intravenously (IV) once every 14 days at a treatment dose during the treatment period. The immune checkpoint inhibitor may be nivolumab administered once every four weeks at a maintenance dose following the treatment period.

The immune checkpoint inhibitor may be administered at a treatment dose between about 50 mg and about 500 mg per subject per day during the treatment period. For example, the immune checkpoint inhibitor may be administered at a treatment dose between about 50 mg and about 500 mg, about 100 mg and about 500 mg, about 150 mg and about 500 mg, about 200 mg and about 500 mg, about 210 mg and about 500 mg, about 220 mg and about 500 mg, about 230 mg and about 500 mg, about 240 mg and about 500 mg, or about 250 mg and about 500 mg per subject per day. The immune checkpoint inhibitor may be administered at a treatment dose of about 50 mg, about 100 mg, about 150 mg, about 200 mg, 220 mg, 240 mg, about 250 mg, about 300 mg, about 350 mg, about 400 mg, about 450 mg, or about 500 mg, per subject per day. In some embodiments, the immune checkpoint inhibitor is nivolumab administered at a treatment dose of about 240 mg during the treatment period.

The immune checkpoint inhibitor may be administered at a maintenance dose between about 300 mg and 500 mg once, twice, three times, or four times every four weeks following the treatment period. For example, the immune checkpoint inhibitor may be administered at a maintenance dose between about 300 mg and about 500 mg, about 350 mg and about 500 mg, about 400 mg and about 500 mg, or about 450 mg and about 500 mg once, twice, three times, or four times every four weeks following the treatment period. In some embodiments, the immune checkpoint inhibitor is administered at a maintenance dose of about 450 mg, about 460 mg, about 470 mg, about 480 mg, about 490 mg, or about 500 mg once, twice, three times, or four times every four weeks following the treatment period. In some embodiments, the immune checkpoint inhibitor is nivolumab administered at a maintenance dose about 480 mg once every four weeks following the treatment period.

The subject may have neuroendocrine tumor or a neuroendocrine carcinoma selected from large cell neuroendocrine carcinoma, extra thoracic small cell carcinoma, poorly differentiated neuroendocrine carcinoma. In some embodiments, the subject has a neuroendocrine carcinoma with a lesion size having the longest diameter between about 8 mm and 55 mm. In some embodiments, the subject has a neuroendocrine carcinoma with a lesion size having the longest diameter between about 10 mm and 50 mm.

The method may be administered to the subject for a treatment period between about four weeks and 24 weeks, following which the subject may demonstrate complete response, partial response, or stable disease following the treatment. The method may be administered to the subject for a treatment period of about 12 weeks, following which the subject may demonstrate complete response, partial response, or stable disease following the treatment. Complete response (CR) comprises disappearance of all known disease determined by two observations not less than four weeks apart. Partial response (PR) comprises 30% or more decrease in the total sum of all measurements as determined by two observations not less than four weeks apart. Stable disease comprises decrease or a less than 20% increase in the total sum of all measurements. The method may reduce the total tumor burden in the subject.

### Pharmaceutical Compositions

Also provided herein is a pharmaceutical composition for treating a cancer in a subject in need thereof, wherein the cancer has been identified as SVV-S based on the expression of the biomarkers described above. The pharmaceutical composition can comprise an SVV composition. The pharmaceutical composition can comprise an immune checkpoint inhibitor composition. The pharmaceutical composition can comprise a pharmaceutically acceptable carrier. The cancer can comprise cancer that is refractory to monotherapy with the checkpoint inhibitor.

The pharmaceutical compositions may be in a form suitable for administration to a subject concomitantly or separately. The pharmaceutical compositions may be suitable for concomitant and/or separate administration of SVV and the immune checkpoint inhibitor. The pharmaceutical compositions may provide SVV and the immune checkpoint inhibitor for administration of both on the same day of the treatment. The pharmaceutical compositions may provide SVV and the immune checkpoint inhibitor for administration of SVV on one day of the treatment and for the administration of the immune checkpoint inhibitor on another day of the treatment. The pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

The pharmaceutical compositions may comprise a vial of SVV comprising between about 2×10³ VG and about 5×10¹⁰ VG of SVV. The pharmaceutical compositions may comprise a vial of the checkpoint inhibitor at a dose between about 50 mg and about 500 mg. For example, the SVV may be at a dose between about 2×10³ VG and about 5×10¹⁰ VG, between about 2×10³ VG and about 4×10¹⁰ VG, between about 2×10³ VG and about 3×10¹⁰ VG, between about 2×10³ VG and about 2×10¹⁰ VG, or between about 2×10⁵ VG and about 2×10¹⁰ VG per vial. The SVV may be at a dose of about 2×10³ VG, about 2×10⁴ VG, about 2×10⁵ VG, about 2×10⁶ VG, about 2×10⁷ VG, about 2×10⁸ VG, about 2×10⁹ VG, about 2×10¹⁰ VG, about 3×10³ VG, about 3×10⁴ VG, about 3×10⁵ VG, about 3×10⁶ VG, about 3×10⁷ VG, about 3×10⁸ VG, about 3×10⁹ VG, about 3×10¹⁰ VG, about 4×10³ VG, about 4×10⁴ VG, about 4×10⁵ VG, about 4×10⁶ VG, about 4×10⁷ VG, about 4×10⁸ VG, about 4×10⁹ VG, about 4×10¹⁰ VG, about 5×10³ VG, about 5×10⁴ VG, about 5×10⁵ VG, about 5×10⁶ VG, about 5×10⁷ VG, about 5×10⁸ VG, about 5×10⁹ VG, or about 5×10¹⁰ VG, per vial. In some embodiments, the SVV is at between 2.2×10⁸ VG and 2.2×10¹⁰ VG per vial, such as about 2.2×10⁸ VG, about 2.2×10⁹ VG, or about 2.2×10¹⁰ VG per vial. The immune checkpoint inhibitor may be at between about 50 mg and about 500 mg per vial. For example, the immune checkpoint inhibitor may at a dose between about 50 mg and about 500 mg, about 100 mg and about 500 mg, about 150 mg and about 500 mg, about 200 mg and about 500 mg, about 210 mg and about 500 mg, about 220 mg and about 500 mg, about 230 mg and about 500 mg, about 240 mg and about 500 mg, or about 250 mg and about 500 mg per vial. The immune checkpoint inhibitor may be at a dose of about 50 mg, about 100 mg, about 150 mg, about 200 mg, 220 mg, 240 mg, about 250 mg, about 300 mg, about 350 mg, about 400 mg, about 450 mg, 480 mg, or about 500 mg, per vial. In some embodiments, the immune checkpoint inhibitor is nivolumab at about 240 mg or 480 mg per vial.

The relative amounts of the active ingredient, the pharmaceutically acceptable carrier, and any additional ingredients in each of the vials of the pharmaceutical composition of the invention will vary, depending upon the identity, size, and condition of the subject treated and further depending upon the route by which the composition is to be administered.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, intratumoral, intravenous, or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit. In some embodiments, the SVV or derivative thereof can be formulated in a natural capsid, a modified capsid, as a naked RNA, or encapsulated in a protective coat.

The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses. When multiple daily doses are used, the unit dosage form may be the same or different for each dose.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs. In one embodiment, the subject is a human or a non-human mammal such as but not limited to an equine, an ovine, a bovine, a porcine, a canine, a feline and a murine. In one embodiment, the subject is a human.

In one embodiment, the compositions are formulated using one or more pharmaceutically acceptable excipients or carriers. In one aspect a pharmaceutical composition is disclosed for treating a cancer in a subject. The pharmaceutical composition comprises an SVV and a pharmaceutical acceptable carrier. Pharmaceutically acceptable carriers, which are useful, include, but are not limited to, glycerol, water, saline, ethanol, and other pharmaceutically acceptable salt solutions such as phosphates and salts of organic acids. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like.

The disclosed composition may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition may include an antioxidant and a chelating agent which inhibit the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition which may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

The pharmaceutical composition disclosed herein may be used in combination with an additional therapeutic agent such as an anti-tumor agent, including but not limited to a chemotherapeutic agent, an anti-cell proliferation agent or any combination thereof. For example, any conventional chemotherapeutic agents of the following non-limiting exemplary classes are included in the invention: alkylating agents; nitrosoureas; antimetabolites; antitumor antibiotics; plant alkyloids; taxanes; hormonal agents; and miscellaneous agents. In another aspect, the pharmaceutical composition disclosed herein may be used in combination with a radiation therapy.

### Administration/Dosing

The SVV is typically administered at a therapeutically effective dose. A therapeutically effective dose refers to that amount of the virus that results in amelioration of symptoms or a prolongation of survival in a patient. Toxicity and therapeutic efficacy of viruses can be determined by standard procedures in cell cultures or experimental animals, e.g., for determining the LD₅₀ (the dose lethal to 50% of the population of animals or cells; for viruses, the dose is in units of vp/kg) and the ED₅₀ (the dose, vp/kg, therapeutically effective in 50% of the population of animals or cells), or the TC₁₀ (the therapeutic concentration or dose allowing inhibition of 50% of tumor cells and can be related to PFU) or the EC₅₀ (the effective concentration, vp/cell, in 50% of the population of animals or cells). The dose ratio between toxic and therapeutic effects is the therapeutic index and it can be expressed as the ratio between LD₅₀ and ED₅₀ or EC₅₀. The dosage of viruses lies preferably within a range of circulating concentrations that include the ED₅₀ or EC₅₀ with little or no toxicity. The dosage may vary within this range depending upon the dosage form employed- and the route of administration utilized.

The SVV may be present in the composition in multidose and single dosage amounts, including, but not limited to between or between about 1×10⁵ and 1×10¹² pfu, 1×10⁶ to 1×10¹⁰ pfu, or 1×10⁷ to 1×10¹⁰ pfu, each inclusive, such as at least, or about at least 1×10⁵, 1×10⁶, 1×10⁷, 1×10⁸, 1×10⁹, 2×10⁹, 3×10⁹, 4×10⁹, 5×10⁹, 6×10⁹, 7×10⁹, 8×10⁹, 9×10⁹, 1×10¹⁰, 1×10¹¹, or 1×10¹² pfu. The SVV may be present in the composition in multidose and single dosage amounts, including, but not limited to between or between about 2×10³ VG and about 5×10¹⁰ VG. The immune checkpoint inhibitor may be present in the composition in multidose and single dosage amounts, including, but not limited to between or between about 50 mg and about 500 mg.

The volume of the composition can be any volume, and can be for single or multiple dosage administration, including, but not limited to, from or from about 0.01 mL to 100 mL, 0.1 mL to 100 mL, 1 mL to 100 mL, 10 mL to 100 mL, 0.01 mL to 10 mL, 0.1 mL to 10 mL, 1 mL to 10 mL, 0.02 mL to 20 mL, 0.05 mL to 5 mL, 0.05 mL to 4 mL, 0.5 mL to 50 mL, or 0.5 mL to 5 mL, each inclusive.

The infectivity of the SVV can be manifested, such as by increased titer or half-life of the oncolytic virus when exposed to a bodily fluid, such as blood or serum. Infectivity can be increased by any amount, including, but not limited to, at least 1.1-fold, 1.2-fold, 1.3-fold, 1.4-fold, 1.5-fold, 1.6-fold, 1.7-fold, 1.8-fold, 1.9-fold, 2.0-fold, 2.5-fold, 3-fold, 4-fold, 5-fold. 6-fold, 7-fold, 8-fold, 9-fold, or 10-fold.

Administration of the compositions of the present invention to a patient subject, preferably a mammal, more preferably a human, may be carried out using known procedures, at dosages and for periods of time effective to treat cancer in the subject. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily, or the dose may be proportionally reduced as indicated by the exigencies of the therapeutic situation.

### Routes of Administration

One skilled in the art will recognize that although more than one route can be used for administration, a particular route can provide a more immediate and more effective reaction than another route.

Routes of administration of the disclosed compositions (containing SVV) include inhalational, oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, subcutaneous, intramuscular, intradermal, intra-arterial, intravenous, intratumoral, intrabronchial, inhalation, and topical administration. Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein. In one embodiment, the SVV treatment and/or treatment with the checkpoint inhibitor comprises an administration route selected from the group consisting of inhalation, oral, rectal, vaginal, parenteral, topical, transdermal, pulmonary, intranasal, buccal, ophthalmic, intra-hepatic arterial, intrapleural, intrathecal, intra-tumoral, intravenal, and any combination thereof.

### Kits

In yet another aspect, also provided herein is a kit for determining a predisposition of an efficacious response to an SVV-based treatment of a cancer in a subject, the kit comprising a reagent for determining the expression level of ANTXR1 and a reagent for determining the expression level of one or more biomarkers described in Table 1 in the cancer from the subject.

In general, kits will comprise a detection reagent that is suitable for detecting the presence of a polypeptide or a nucleic acid, such as cDNA or mRNA of interest (e.g. a biomarker described in Table 1).

In another embodiment, the kit comprises a panel of probe sets or antibodies. Preferred probe sets may be designed to detect the expression level of ANTXR1 as well as one of the biomarkers described herein and provide information about the efficacy of an SVV-based cancer treatment. Probe sets are particularly useful because they are smaller and cheaper than probe sets that are intended to detect as many polynucleotides as possible in a particular genome. As provided herein, the probe sets or the antibodies are targeted at the detection of polynucleotides or polypeptides that are informative about ANTXR1 and one or more of the biomarkers described herein in cancer cells or tissues. Probe sets or antibodies may also comprise a large or small number of probes that detect polynucleotides or peptides that are not informative about cancer. Such probes are useful as controls and for normalization (e.g., spiked-in markers). Probe sets or antibodies may be a dry mixture or a mixture in solution. In some embodiments, probe sets or antibodies can be affixed to a solid substrate to form an array of probes or antibodies. The probes may be antibodies, or nucleic acids (e.g., DNA, RNA, chemically modified forms of DNA and RNA), LNAs (Locked nucleic acids), or PNAs (Peptide nucleic acids), or any other polymeric compound capable of specifically interacting with the peptides or nucleic acid sequences of interest.

It is contemplated that kits may be designed for isolating and/or detecting peptides (e.g. ANTXR1, known cancer markers, immune activators, or apoptotic proteins) or nucleic acid sequences in essentially any sample (e.g., leukemic blood, tumor cells, tumor tissue, etc.), and a wide variety of reagents and methods are, in view of this specification, known in the art.

In further embodiments a kit is provided for treating or ameliorating a cancer, as described elsewhere herein wherein the kit comprises: a) a compound or compositions as described herein; and b) an additional agent or therapy as described herein. The kit can further include instructions or a label for using the kit to treat or ameliorate the cancer. In yet other embodiments, the invention extends to kits assays for a given cancer (such as, but not limited to, small-cell lung cancer or triple negative breast cancer), as described herein. Such kits may, for example, contain the reagents from PCR or other nucleic acid hybridization technology (microarrays) or reagents for immunologically based detection techniques (e.g., ELISpot, ELISA).

### EXAMPLES

The invention is now described with reference to the following Examples. These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples, therefore, specifically point out the preferred embodiments of the present invention and are not to be construed as limiting in any way the remainder of the disclosure.

### Example 1—Identification of SVV Biomarker Profiles

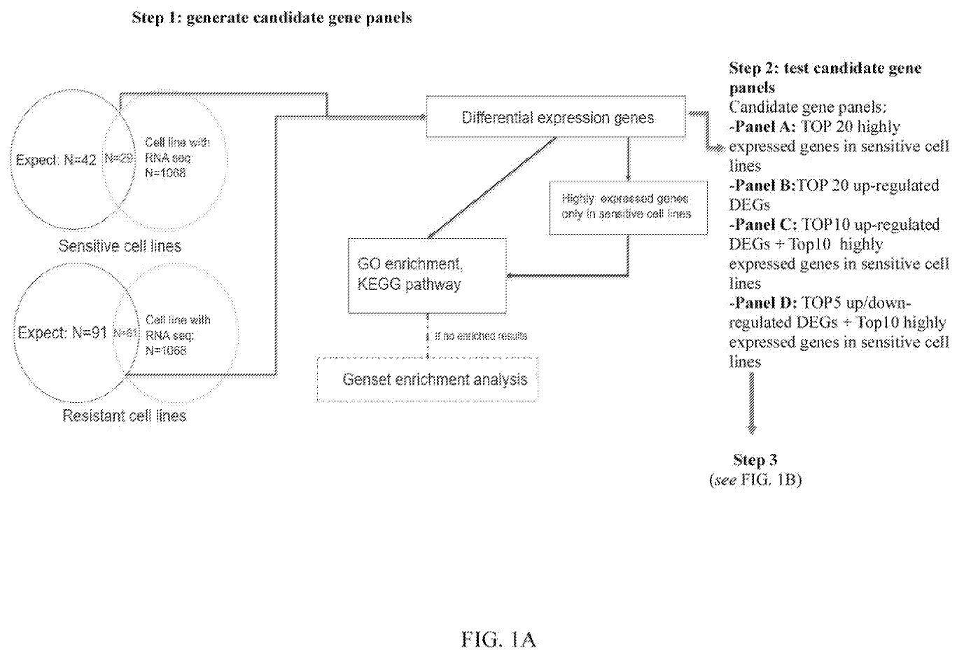

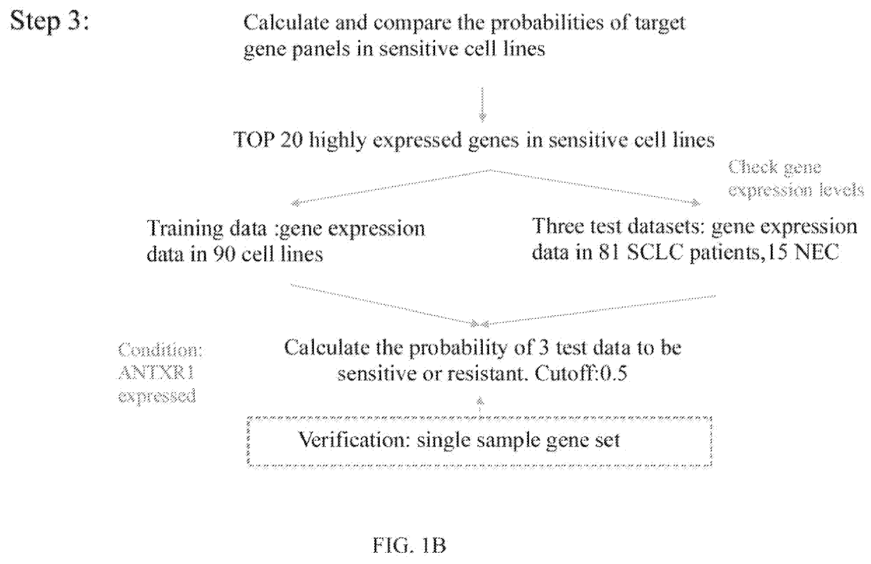

This Example provides a brief overview as to how the SVV biomarker profiles disclosed in the instant application were generated. The process of generating the SVV biomarker profiles is summarized in FIG. 1A and FIG. 1B.

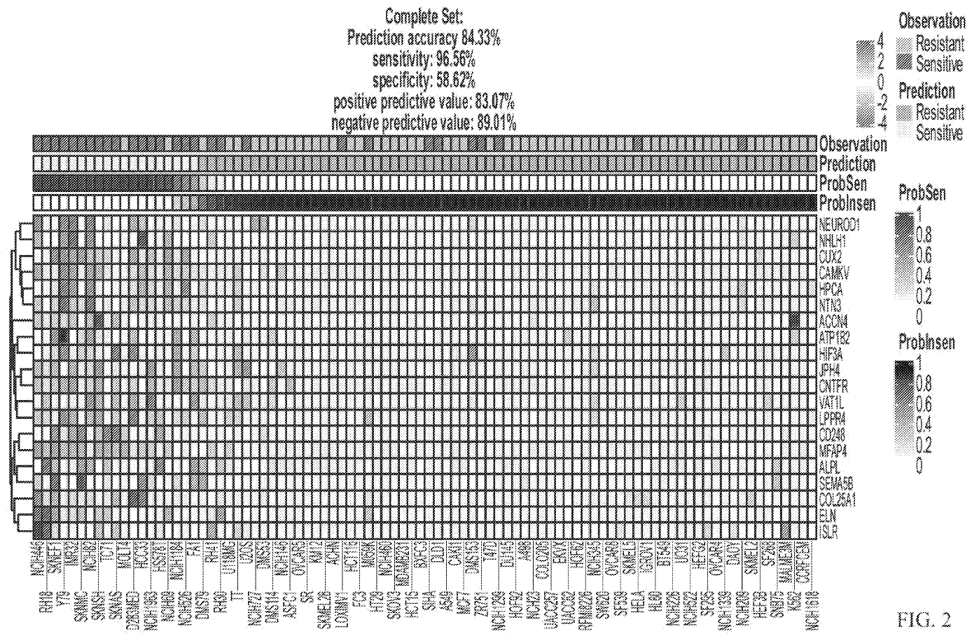

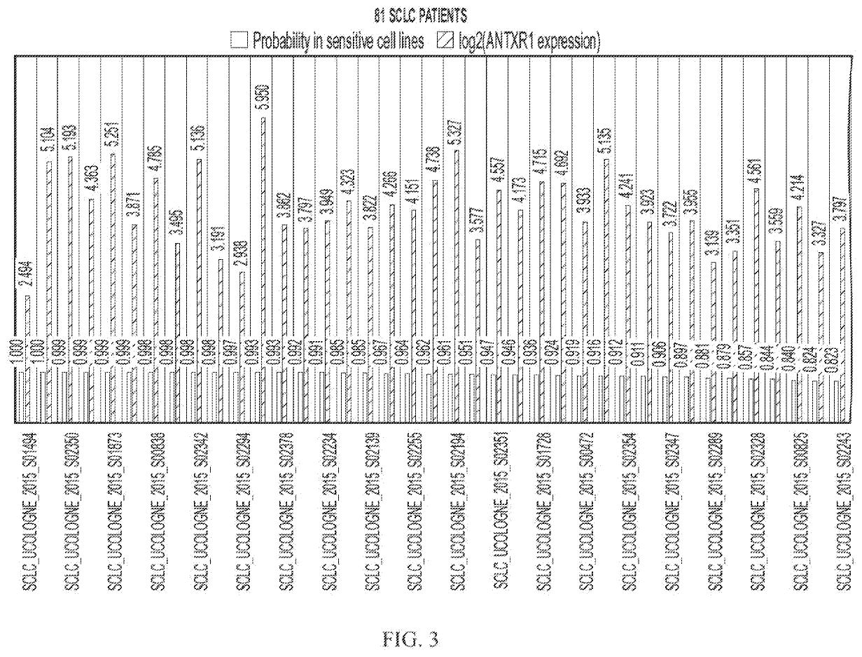

The starting point was 29 human cell lines that were known to be sensitive/responsive to treatment with Seneca Valley Virus (“SVV-Sensitive” or “SVV-S”) and 61 cell lines that were known to be resistant to treatment to with Seneca Valley Virus (“SVV-Resistant” or “SVV-R”) (see FIG. 1A). Approximately 20,000 genes were analyzed for each cell line and genes differentially expressed in SVV-R and SVV-S cell lines were identified. Using the workflow shown in FIG. 1A, RNAseq results from 90 SVV-S and SVV-R cell lines were tested to identify and select highly expressed genes and differentially expressed genes (DEGs) in SVV-S cell lines compared to SVV-R cell lines. The candidate gene panel A (top 20 highly expressed genes in sensitive cell lines) is used in FIG. 2. Additional panels of top 20 up-regulated DEGs (Panel B); top 10 up-regulated DEGs+Top10 highly expressed genes in sensitive cell lines (Panel C); and top 5 up/down-regulated DEGs+Top10 highly expressed genes in sensitive cell lines (Panel D) were developed. SVV-S(SVV sensitive, also termed herein as SVV permissive) cells lines were those which had EC50 of less than 1000 vp/cell and SVV-R (SVV resistant, also termed herein as SVV non-permissive) cell lines were those which had EC50 of greater than 10,000 vp/cell.

Based on this analysis, a number of candidate gene panels that could discriminate between SVV-S and SVV-R cells depending on different criteria were identified. Panel A (Table 2) shows the TOP 20 highly expressed genes in sensitive cell lines (SVV-S).

TABLE 2
Panel A (TOP 20 highly expressed genes in SVV-S)

| | | | |
|---|---|---|---|
| ELN | ATP1B2 | HPCA | ACCN4 |
| CD248 | NEUROD1 | ISLR | JPH4 |
| COL25A1 | VAT1L | NHLH1 | HIF3A |
| ALPL | MFAP4 | CUX2 | CNTFR |
| SEMA5B | LPPR4 | CAMKV | NTN3 |

FIG. 2 shows the results of this analysis (20-gene Panel A across a training set of cell lines) for Panel A. As is evident from FIG. 2, there is dichotomous segregation between SVV-S and SVV-R cell lines. Specifically, FIG. 2 shows the twenty gene expression profiles for all 90 cell lines. In FIG. 2, the SVV-S lines are separated from the SVV-R lines (in the Observation line, black indicates highly expressed genes whereas pale gray indicates genes expressed at low levels). The outcome measures are: prediction accuracy; sensitivity; specificity; positive predictive value; negative predictive value. The prediction accuracy to distinguish SVV-Sensitive and SVV-Resistant cell lines was 84.33%.

There was substantial overlap between genes identified using Panel A-D. The candidate biomarker panels were then tested against: (1) RNAseq gene expression data from 81 patients with small cell lung cancer (SCLC); (2) RNA expression profiles from 15 neuroendocrine carcinoma patients (NEC); and (3) 69 patient-derived xenograft (PDX) models in the Crown Bioscience database. Initially, a probability of being SVV-Sensitive as 50% was used but then the stringency was increased to 75% in subsequent analyses. Because TEM8/ANTXR1 is the receptor for SVV, this was included in the biomarker panel as TEM8 is a pre-requisite for SVV infection. By itself, TEM8 expression is not a good discriminator between SVV-S and SVV-R cell lines.

Testing with Panel A

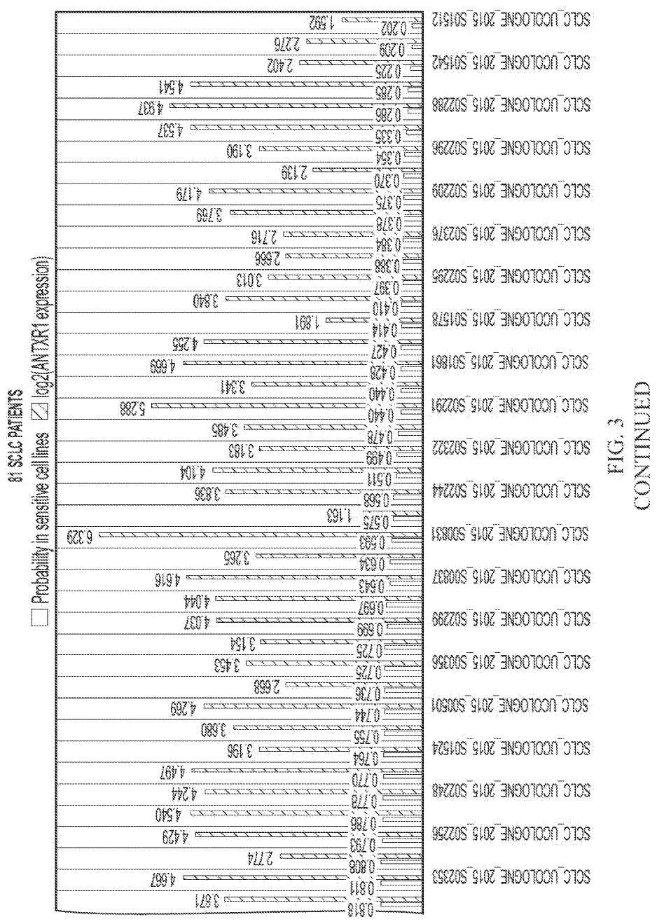

The results of this testing for Panel A is shown in FIG. 3-5. FIG. 3 shows the results of RNAseq gene expression data from 81 patients with small cell lung cancer (SCLC) compared against Panel A. Specifically, FIG. 3 shows the probabilities of Panel A in sensitive SCLC cell lines and gene ANTXR1 expression levels. Based on this testing, 74% of patients were predicted as SVV-sensitive with ANTXR1 expressed (61 of results are more than 0.5). The correlation between the log2(ANTXR1 expression) and the probability to be sensitive to SVV was determined to be 0.35<0.5. Log2 ANTXR1 is shown with hatched bars and probability of SVV sensitivity is shown with white bars. 42/81 (52%) of SCLC patient samples showed >80% probability of being SVV-S and 61/81 (74%) of SCLC patient samples showed >50% probability of SVV-S. ANTXR1 is over-expressed in all 81 samples.

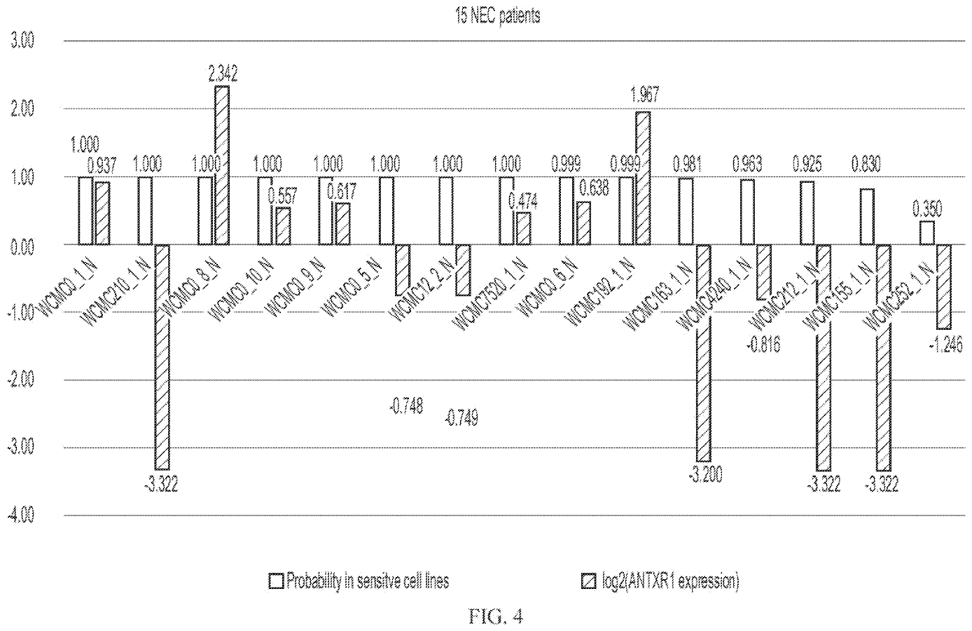

FIG. 4 shows the results of RNA expression profiles from 15 neuroendocrine carcinoma patients (NEC) compared against Panel A. Specifically, FIG. 4 shows the probabilities of Panel A in sensitive NEC cell lines and gene ANTXR1 expression levels. ANTXR1 gene was observed to be only expressed in 7 samples. 93% of the samples were predicted as SVV-sensitive (13 of results are more than 0.5). 7 samples are predicted as sensitive when ANTXR1 is expressed. Log2 ANTXR1 is shown with hatched bards and probability of SVV sensitivity is shown with white bars. All but one (93%) NEC patient samples showed a SVV-S biomarker genotype, however ANTXR1 gene is only expressed in 7 samples. Seven out of 15 patient samples (47%) were SVV-S when adding ANTXR1 expression.

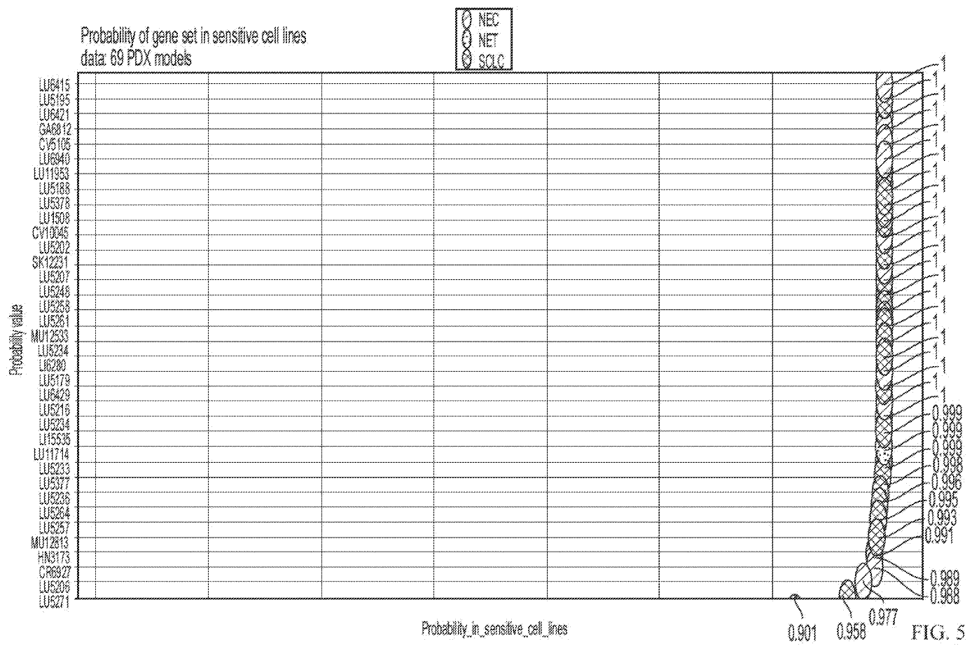

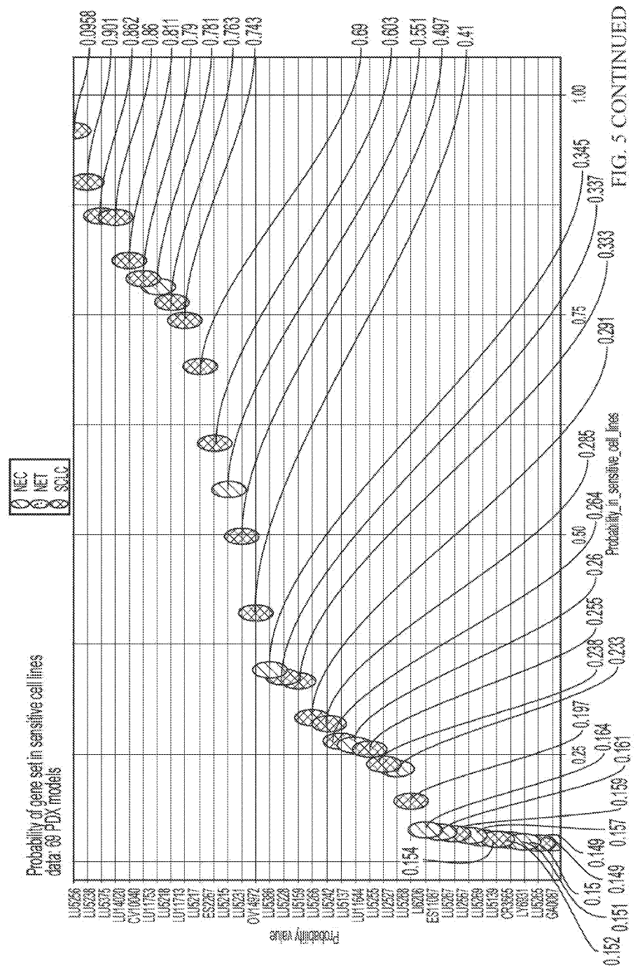

FIG. 5 shows the results of 69 PDX models compared against Panel A. 67% were predicted as SVV-sensitive with (46 of results are more than 0.5). In the PDX model, 45 of the cancers were SCLC; 23 of the cancers were NEC; and one of the cancers was a neuroendocrine tumor (NET). For the SCLC patients in the PDX model datasets, expression levels of gene COL25A1, CUX2, LPPR4 were observed to be low in the half of samples. It was postulated that this difference might be due to the biology of these human tumors (different from cell lines), or may be because Panel A is not optimal. Probability of SVV-sensitivity is shown in each colored ball. Most (63%, 43/68) NEC and SCLC PDX samples showed a probability of SVV-S >70%. The single NET sample showed a 99% probability of being SVV-S. 65% of samples were SCLC; 33% were NEC and 1.45% were NET.

Testing with Panel 2

To optimize the robustness of the biomarker panel A and reduce the influence by low expressed genes, these three genes were replaced with EFS, NEFH, and SOX11 to generate Panel 2, which is shown in Table 3 below.

TABLE 3
Panel 2

| | | | |
|---|---|---|---|
| ELN | ATP1B2 | HPCA | ACCN4 |
| CD248 | NEUROD1 | ISLR | JPH4 |
| EFS | VAT1L | NHLH1 | HIF3A |
| ALPL | MFAP4 | SOX11 | CNTFR |
| SEMA5B | NEFH | CAMKV | NTN3 |

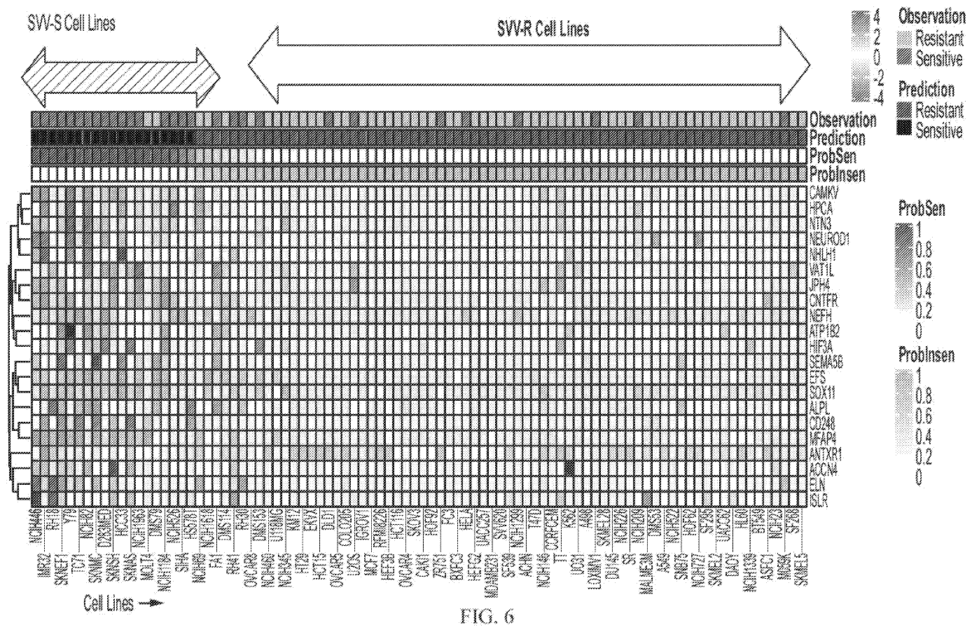

The expression of the genes of Panel 2 and ANTRX1 in SVV-S and SVV-R cell lines are shown in FIG. 6. Three of the 20 genes in candidate gene Panel A were weakly expressed in SCLC patients and PDX models datasets. The three genes were replaced by EFS, NEFH and SOX11 genes and these 20 genes were selected as the new gene panel (gene Panel 2). For the new gene Panel 2, the prediction accuracy to distinguish sensitive and insensitive cell lines was 83.44%. The prediction accuracy was 84.22% when ANTXR1 gene was added as composite biomarker geneset.

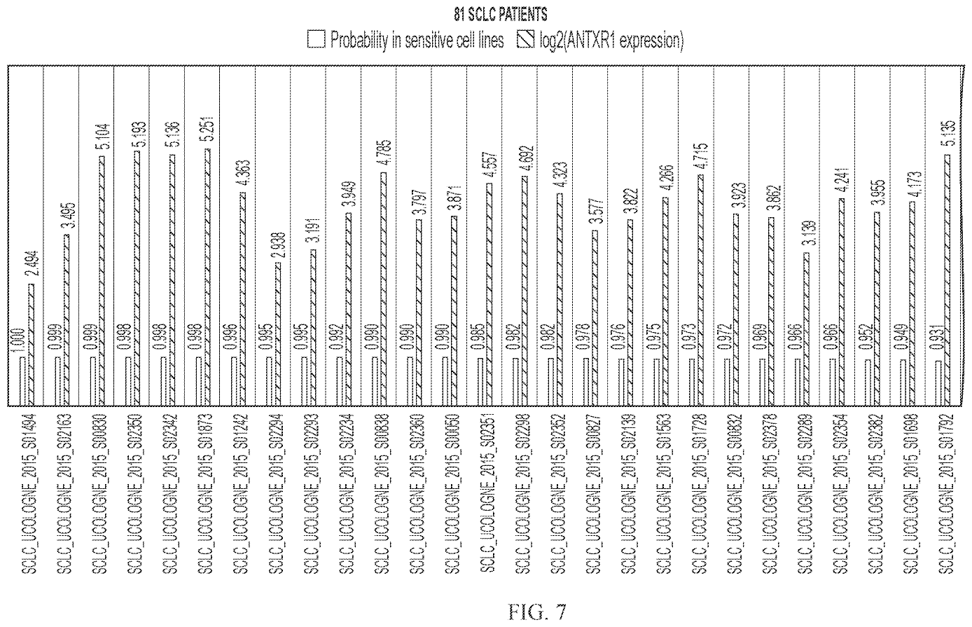

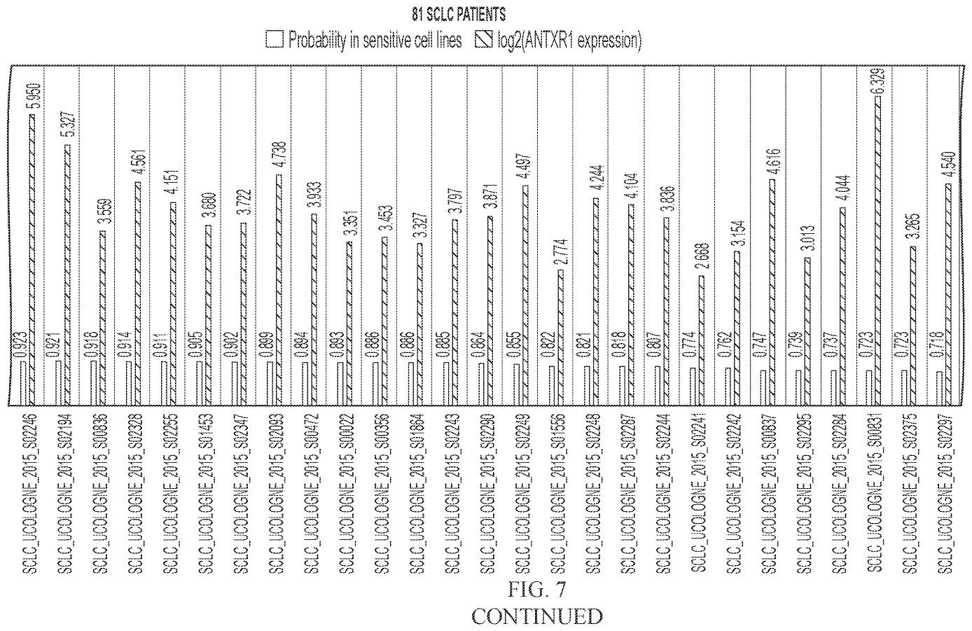

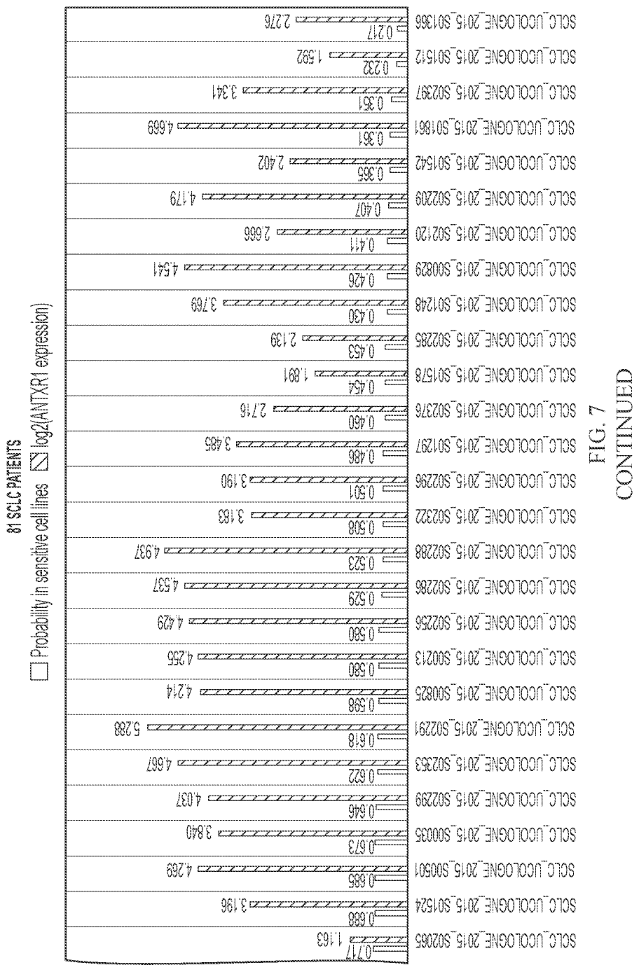

Similar testing as for Panel A was conducted with Panel 2. The results of this testing are shown in FIGS. 7-9.

FIG. 7 shows the results of RNAseq gene expression data from 81 patients with small cell lung cancer (SCLC) compared against Panel 2. Specifically, FIG. 7 shows the probabilities of panel 2 in SVV-S lines and ANTXR1 gene expression levels. ANTXR1 gene expression was observed in over 81 samples. 84% were predicted as SVV-sensitive with ANTXR1 expressed (68 of results are more than 0.5). The correlation between the log2(ANTXR1 expression) and the probability to be sensitive was observed to be 0.35<0.5. Log2 ANTXR1 is shown with hatched bars and probability of SVV sensitivity is shown with white bars. 46/81 (57%) of SCLC patient samples showed >80% probability of being SVV-S.

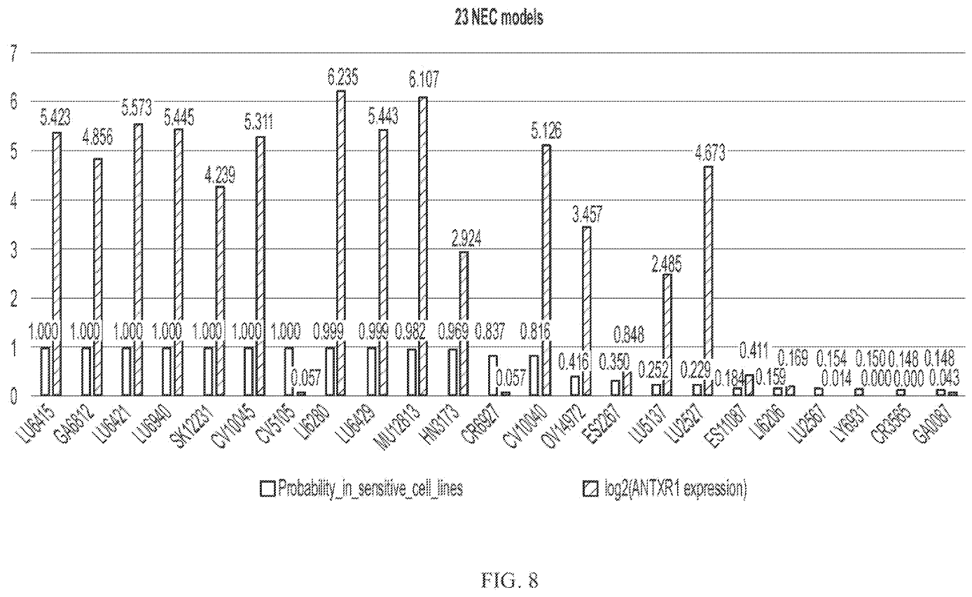

FIG. 8 shows the results of RNA expression profiles from 23 neuroendocrine carcinoma patients (NEC) PDX samples compared against Panel 2. Specifically, FIG. 8 shows the probabilities of panel 2 in SVV-S lines and ANTXR1 gene expression levels. ANTRX1 gene expression was observed in 21 samples. 57% were predicted as SVV-sensitive with ANTXR1 expressed (13 of results are more than 0.5). Log2 ANTXR1 is shown with hatched bars and probability of SVV sensitivity is shown with white bars.

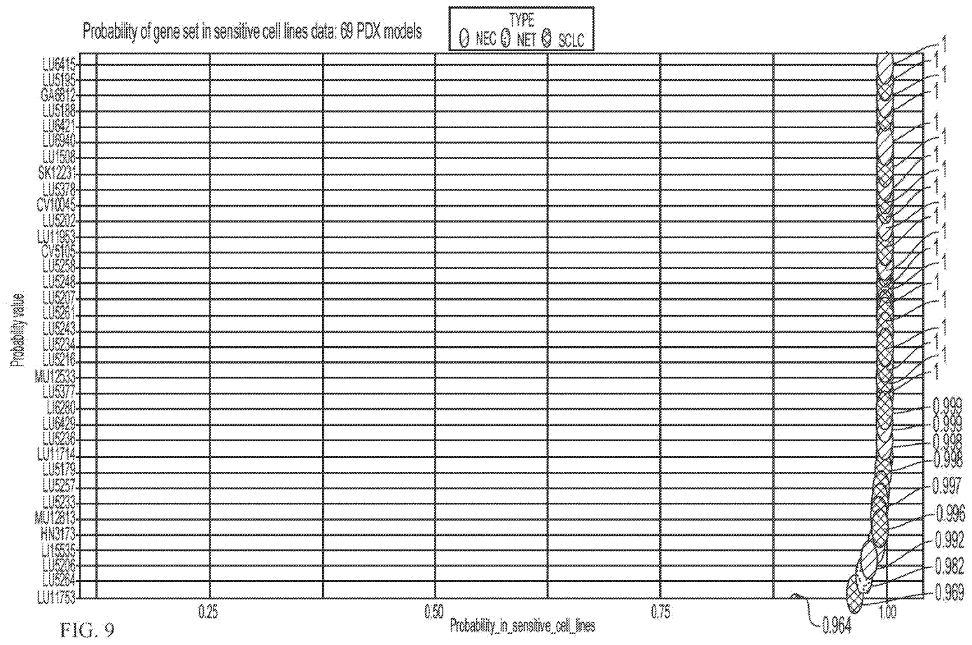

FIG. 9 shows the results of 69 PDX models comprising NEC, NET and SCLC samples compared against Panel 2. Probability of SVV-sensitivity is shown in each colored ball. Most (44/69) NEC and SCLC PDX samples showed a probability of SVV-S >70%. The single NET sample showed a 96% probability of being SVV-S.

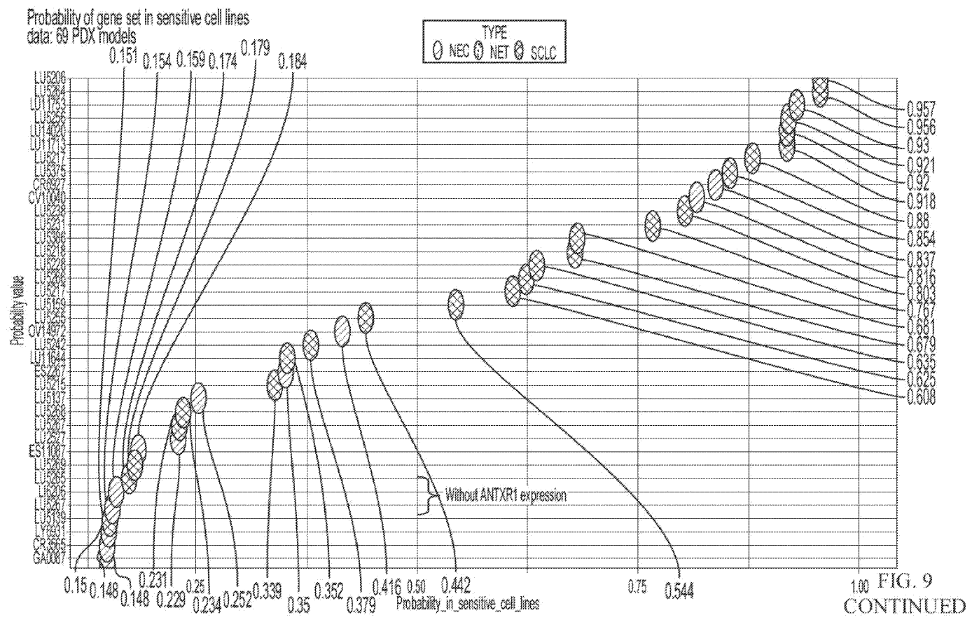

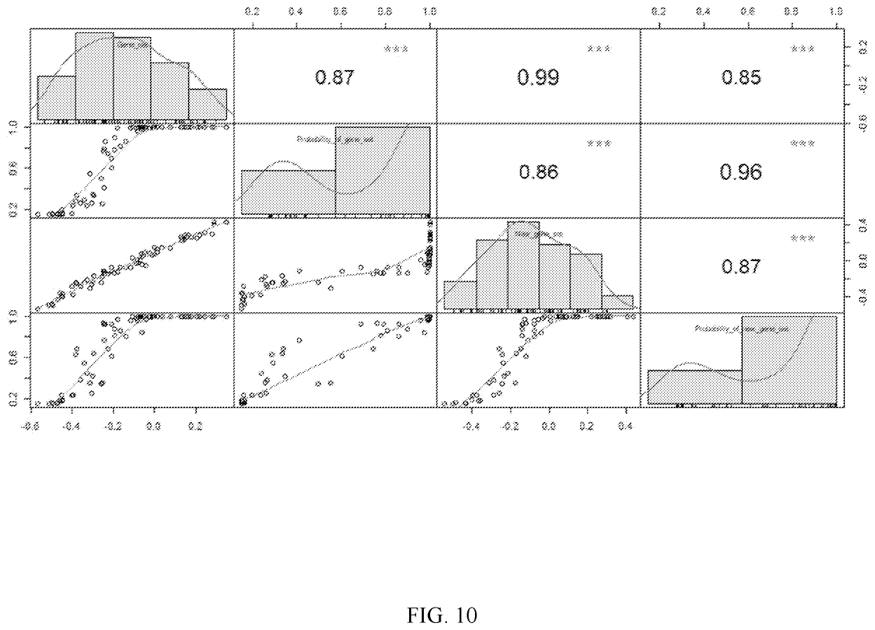

The results were also verified by performing single sample gene set enrichment analysis (ssGSEA) of the selected Panel 2 genes for the 69 PDX models (see FIG. 10). The correlation between the predicted probability and ssGSEA enrichment score for PDX models was 0.87. To verify the prediction results, the ssGSEA score of new gene panel for 69 PDX models was computed by gene set variation analysis (GSVA) (Hänzelmann, et al. GSVA: gene set variation analysis for microarray and RNA-Seq data. BMC Bioinformatics 14(7):1-15 (2013)) and plots were drawn via ‘PerformanceAnalytics’ package. Correlation analysis between ssGSEA scores and probabilities of sensitivity based on selected gene panel for 69 PDX models revealed high concordance (87%) on the prediction results. A-B: ssGSEA scores and probabilities of sensitivity based on the first 20-gene panel (gene set A); C-D: ssGSEA scores and probabilities of sensitivity based on new 20-gene panel containing EFS, NEFH and SOX11 genes (new_gene_set). Spearman correlation coefficients are shown, and statistical significance are represented by: * (p≤0.05), ** (p<0.01), *** (p≤0.001).

Two strong positive correlations between two enrichment results and probability results for both Panel A and Panel 2 were observed. It seems genes COL25A1, CUX2 and LPPR4 may be retained. Therefore, the prediction results appear to be reasonable and reliable.

### Summary of the Testing of Panel a and Panel 2

The SVV-sensitivity in 81 SCLC patients, 15 NEC patients and 69 PDX models was tested by using two 20-gene panels identified from cell line data (Panel A and Panel 2). Using Panel A, 113 samples out of 165 human samples were predicted as SVV-sensitive as follows: (a) NEC_sensitive/NEC=55%; (b) SCLC_sensitive/SCLC=72%; and (c) only one NET sample which was predicted as sensitive. Similar results in the prediction were observed by testing Panel 2, which only differs from Panel A by three genes. The prediction was confirmed by ssGSEA with high correlation of probability of sensitivity and enrichment score in 69 PDXs.

### Testing of Panel a and Panel 2 on Normal Organoid Samples

Panel A and Panel 2 were also tested on normal non-cancer tissue (normal organoid samples). The result of this testing is shown in FIG. 11 and FIG. 12 (results of testing 20-gene panel A across 25 organoid samples from normal tissues). For each of these tests, there were not any obvious differences and positive results in the twenty-five organoid samples derived from normal tissues for the probability to be SVV-S. In FIG. 11, the probability of SVV-sensitivity is shown with white bars and the log2 ANTXR1 levels are shown with hatched bar. All the normal samples showed <0.16% probability of being SVV-S. In FIG. 12, the probability of SVV-sensitivity is shown with white bars and the log2 ANTXR1 levels are shown with hatched bars. All the normal samples showed <0.12% probability of being SVV-S.

### Testing of Additional Panels

The testing of SCLC, NEC, and PDX samples were repeated with different gene combinations. Specifically, the biomarker genes shown in Table 4 below were tested.

TABLE 4
Additional Panels tested

| Panel 2.1 | Panel 2.2 | Panel 3 | Panel 4 |
|---|---|---|---|
| ELN | ELN | COL25A1 | ATP2B3 |
| CD248 | CD248 | CSF2RA | COL25A1 |
| EFS | EFS | NCAM1 | HAND2 |
| ALPL | ALPL | NDN | RUNX1T1 |
| SEMA5B | SEMA5B | ST8SIA2 | PCDH8 |
| | ATP1B2 | TSPYL5 | CNTN4 |
| | NEUROD1 | | EFS |
| | VAT1L | | SOX11 |
| | MFAP4 | | TSPYL5 |
| | NEFH | | GRIK3 |
| | | | NDN |
| | | | ST8SIA2 |
| | | | CSAG3 |

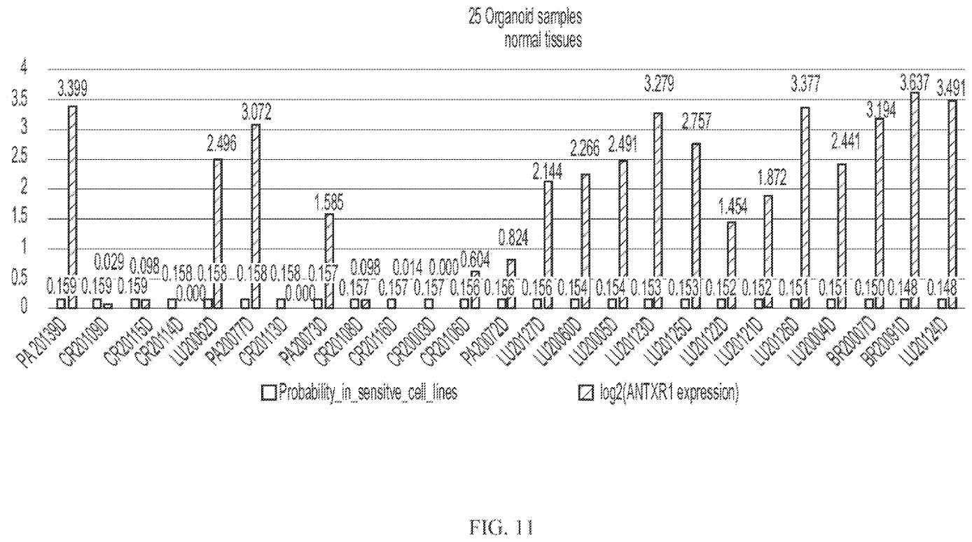

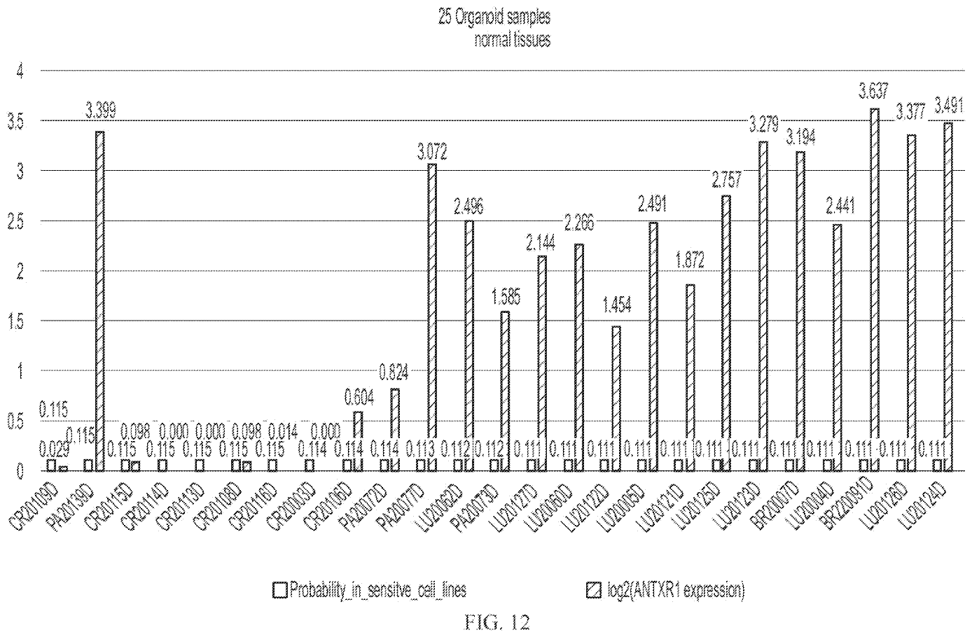

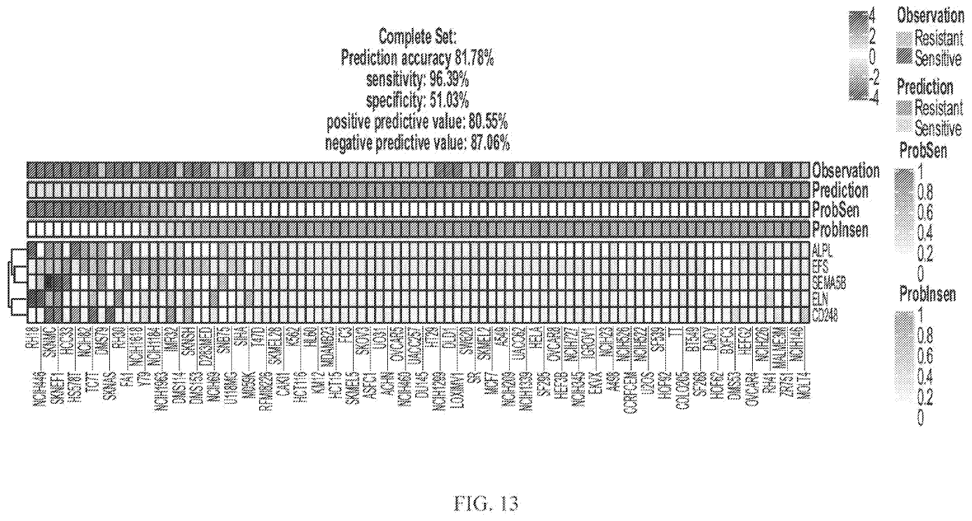

The results of this testing are shown in FIGS. 13-16.

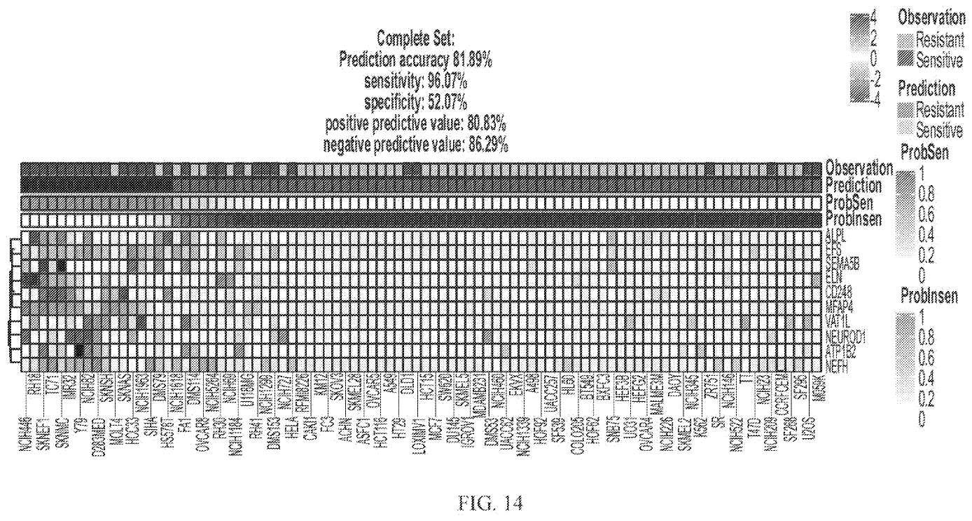

The gene expression of Panel 2.1 genes, which are the top 5 genes from Panel 2, in SVV-R and SVV-S cell lines is shown in FIG. 13. Compared to Panel 2, good negative predictive value (NPV) but worse overall accuracy and positive predictive value (PPV) were observed for Panel 2.1. The prediction accuracy to distinguish SVV-Sensitive and SVV-Resistant cell lines was 81.78%. The gene expression of Panel 2.2 genes, which are the top 10 genes from Panel 2, in SVV-R and SVV-S cell lines is shown in FIG. 14. Similar to Panel 2.1, when compared to Panel 2, good NPV but worse overall accuracy and PPV were observed for Panel 2.2. The prediction accuracy to distinguish SVV-Sensitive and SVV-Resistant cell lines was 81.89%.

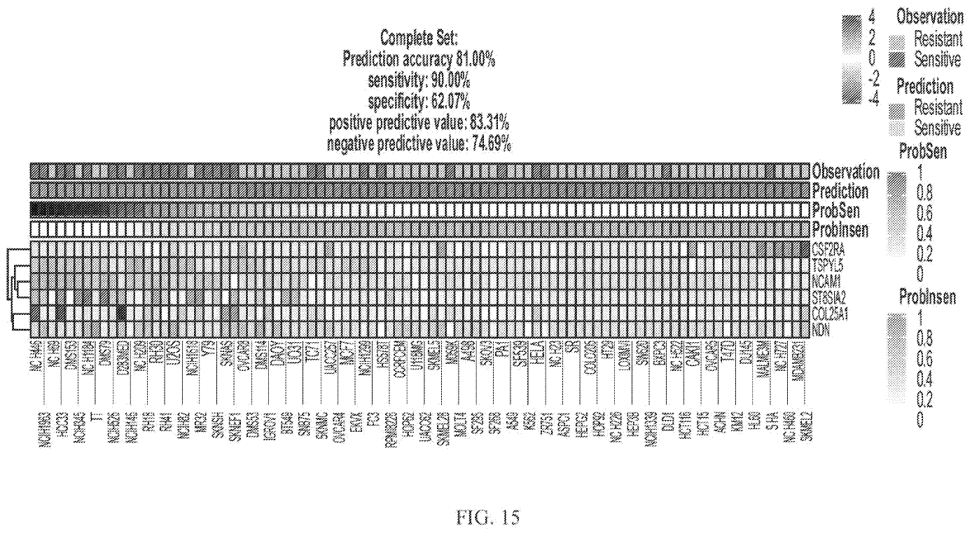

The gene expression of the six Panel 3 genes (6-genes from panel 2 across training set of cell lines) in SVV-R and SVV-S cell lines is shown in FIG. 15. Compared to Panel 2, worse PPV/NPV and overall accuracy were observed for Panel 3. The prediction accuracy to distinguish SVV-Sensitive and SVV-Resistant cell lines was 81.00%. The gene expression of the thirteen Panel 4 genes (13-gene panel from gene panel 2 across training set of cell lines) in SVV-R and SVV-S cell lines is shown in FIG. 16. The prediction accuracy to distinguish SVV-Sensitive and SVV-Resistant cell lines was 76.00%. Similar to Panel 3, when compared to Panel 2, worse PPV/NPV and overall accuracy were observed for Panel 4.

### Additional Testing of Panel 2

In addition, the 20-gene Panel 2 was tested against organoid samples (357 organoid cancers, 25 normal organoids) and 265 TCGA sarcoma samples. The analyzed sarcoma samples from the TCGA are shown in Table 5 below.

TABLE 5
List of analyzed sarcoma samples from TCGA

| ONCOTREE_{—} | | |
|---|---|---|
| CODE | Detail | Count |
| CONTROL | Normal | 2 |
| DDLS | Dedifferentiated Liposarcoma | 57 |
| DES | Desmoid/Aggressive Fibromatosis | 2 |
| LMS | Leiomyosarcoma | 104 |
| MFH | Undifferentiated Pleomorphic Sarcoma/ | 49 |
| | Malignant Fibrous Histiocytoma/High Grade | |
| | Spindle Cell Sarcoma | |
| MFS | Myxofibrosarcoma | 25 |
| MPNST | Malignant Peripheral Nerve Sheath Tumor | 9 |
| PLLS | Pleomorphic Liposarcoma | 2 |
| SARCNOS | Soft Tissue Sarcoma | 1 |
| SOFT_TISSUE | Soft Tissue | 4 |
| SYNS | Synovial Sarcoma | 10 |

In all of these sarcoma samples, ANTRX1/TEM8 expression was observed to be high.

The testing of 265 sarcoma patient RNA from TCGA showed that 95% of sarcoma samples would be defined as SVV-S using a probability cutoff of 75% (0.75). The testing included testing for ANTXR1.

### ANTRX1 Expression in Sarcomas

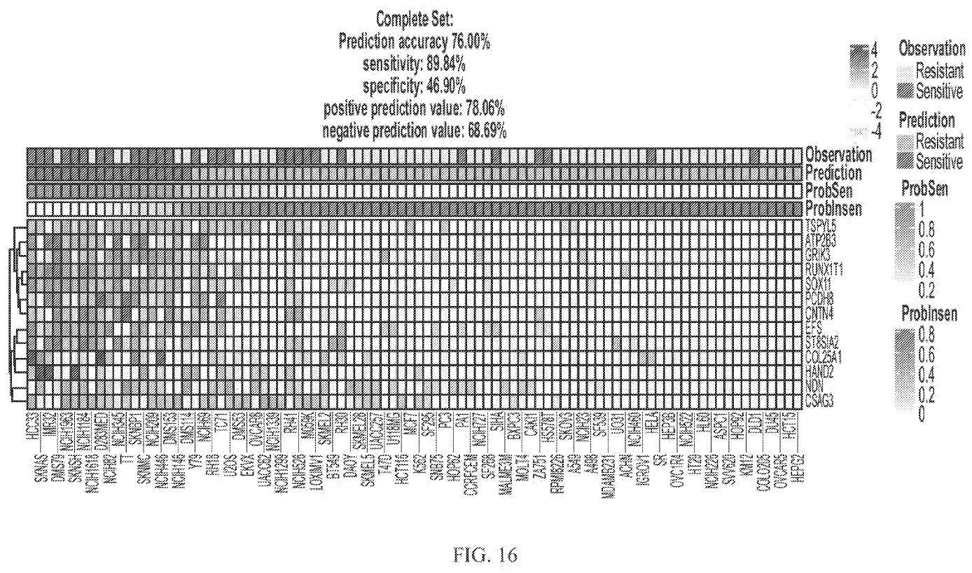

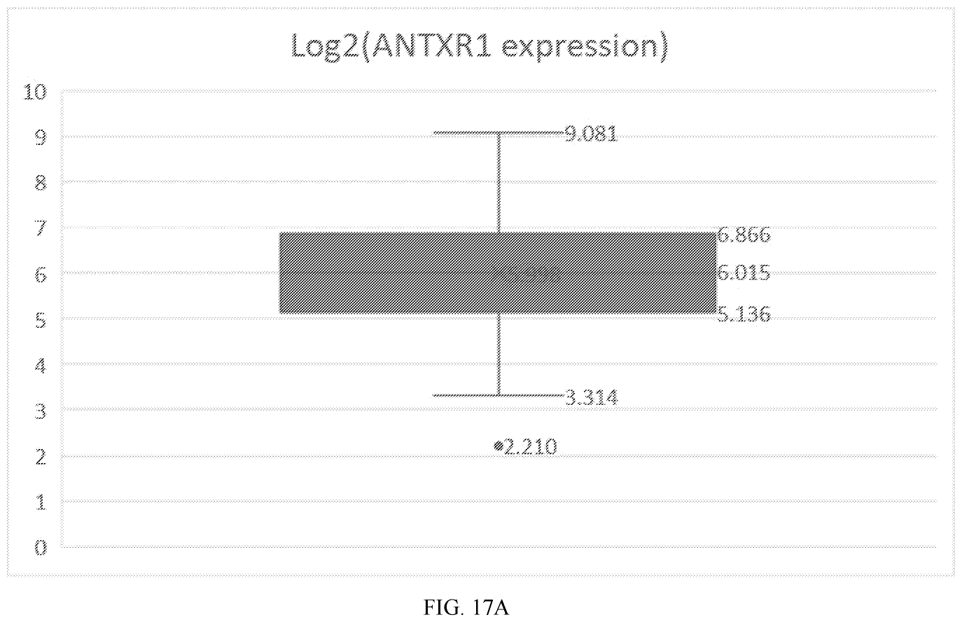

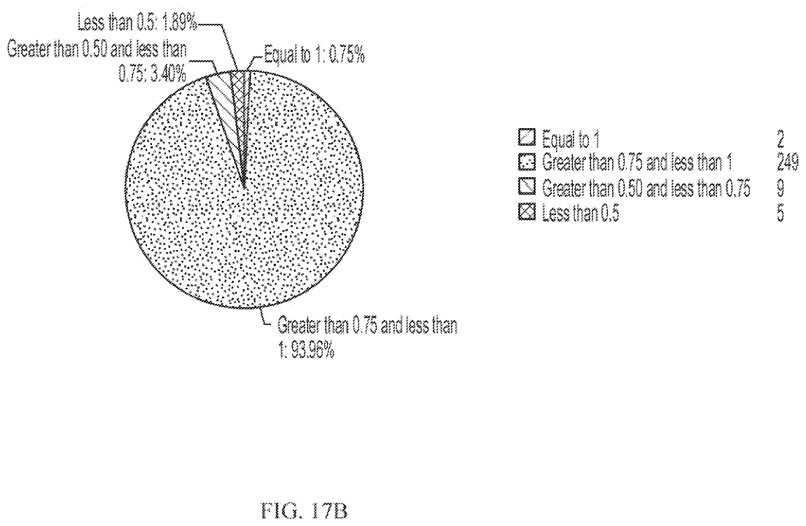

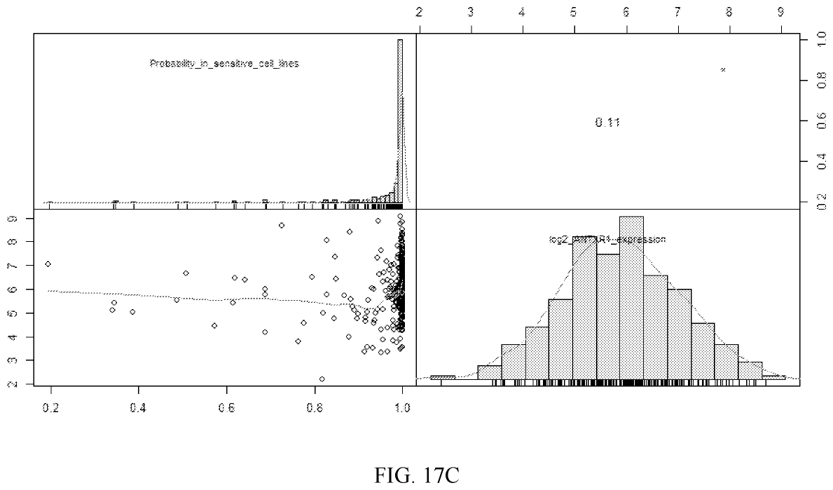

In order to further improve the accuracy for the various biomarker panels, TEM8/ANTRX1 expression was analyzed in the test data. It was observed that ANTXR1 is highly expressed in all sarcomas. The results of this testing are shown in FIG. 17A-17C. No correlation was observed between log2(ANTXR1 expression) and probability of sensitivity in sarcoma: 0.11<0.5.

### Further Statistical Analysis

Based on the testing discussed above, further statistical analysis was conducted. Specifically, the statistical analysis was conducted using the genes from Panel 2 (ELN, CD248, EFS, ALPL, SEMA5B, ATP1B2, NEUROD1, VATIL, MFAP4, NEFH, HPCA, ISLR, NHLH1, SOX11, CAMKV, ACCN4, JPH4, HIF3A, CNTFR, and NTN3).

To conduct this analysis, the genes were sorted by: t-statistic value between sensitive vs. resistant groups; log2 fold change value between sensitive vs. resistant groups; and median expression value for SVV-sensitive group. The top 5-20 genes were all evaluated for prediction performance on cell line data. The results of this analysis in Table 6 below.

The top 7 genes that were sorted by t statistics with adding ANTXR1 as an 8-gene panel—EFS, MFAP4, NTN3, SOX11, NEFH, CNTFR, JPH4, and ANTXR1—showed the best prediction accuracy 85.89% (Table 6).

TABLE 6
Biomarker panels as determined by statistical analysis

| | | | | | Positive | Negative |
|---|---|---|---|---|---|---|
| | | Prediction | | | predictive | predictive |
| Panel name | Genes | accuracy | Sensitivity | Specificity | value | value |
| Tstatistic + | EFS, | 0.8588888 | 0.9163934 | 0.7379310 | 0.8803149 | 0.8075471 |
| ANTXR1 | MFAP4, | 89 | 43 | 34 | 61 | 7 |
| | NTN3, | | | | | |
| | SOX11, | | | | | |
| | NEFH, | | | | | |
| | CNTFR, | | | | | |
| | JPH4, | | | | | |
| | ANTXR1 | | | | | |
| Tstatistic | EFS, MFAP4 | 0.8522222 | 0.9163934 | 0.7172413 | 0.8720748 | 0.8030888 |
| | NTN3, | 22 | 43 | 79 | 83 | 03 |
| | SOX11, | | | | | |
| | NEFH, | | | | | |
| | CNTFR | | | | | |
| Log2FC + | ELN, CD248, | 0.8522222 | 0.9459016 | 0.6551724 | 0.8522895 | 0.8520179 |
| ANTXR1 | ALPL, | 22 | 39 | 14 | 13 | 37 |
| | SEMA5B, | | | | | |
| | ATP1B2, | | | | | |
| | NEUROD1 | | | | | |
| | VAT1L, | | | | | |
| | MFAP4, | | | | | |
| | HPCA, ISLR, | | | | | |
| | NHLH1, | | | | | |
| | CAMKV, | | | | | |
| | ACCN4, | | | | | |
| | ANTXR1 | | | | | |
| MIX(rank by | ELN, CD248, | 0.8466666 | 0.9262295 | 0.6793103 | 0.8586626 | 0.8140495 |
| the 20 genes | EFS, ALPL, | 67 | 08 | 45 | 14 | 87 |
| panel used) + | SEMA5B, | | | | | |
| ANTXR1 | ATP1B2, | | | | | |
| | NEUROD1 | | | | | |
| | VAT1L, | | | | | |
| | MFAP4, | | | | | |
| | NEFH, | | | | | |
| | HPCA, ISLR, | | | | | |
| | NHLH1, | | | | | |
| | SOX11 | | | | | |
| | CAMKV | | | | | |
| | ANTXR1 | | | | | |
| Median + | SOX11, EFS, | 0.8455555 | 0.9180327 | 0.6931034 | 0.8628659 | 0.8007968 |
| ANTXR1 | NEFH, | 56 | 87 | 48 | 48 | 13 |
| | MFAP4 | | | | | |
| | CNTFR | | | | | |
| | ANTXR1 | | | | | |
| MIX(rank by | ELN, CD248, | 0.8433333 | 0.9180327 | 0.6862068 | 0.8602150 | 0.7991967 |
| the 20 genes | EFS, ALPL, | 33 | 87 | 97 | 54 | 87 |
| panel used) | SEMA5B, | | | | | |
| | ATP1B2, | | | | | |
| | NEUROD1 | | | | | |
| | VAT1L, | | | | | |
| | MFAP4, | | | | | |
| | NEFH, | | | | | |
| | HPCA, ISLR, | | | | | |
| | NHLH1, | | | | | |
| | SOX11 | | | | | |
| Log2FC | ELN, CD248, | 0.84 | 0.9278688 | 0.6551724 | 0.8498498 | 0.8119658 |
| | ALPL, | | 52 | 14 | 5 | 12 |
| | SEMA5B, | | | | | |
| | ATP1B2, | | | | | |
| | NEUROD1 | | | | | |
| | VAT1L, | | | | | |
| | MFAP4, | | | | | |
| | HPCA, ISLR, | | | | | |
| | NHLH1 | | | | | |
| Median | SOX11, EFS, | 0.8377777 | 0.9016393 | 0.7034482 | 0.8647798 | 0.7727272 |
| | NEFH, | 78 | 44 | 76 | 74 | 73 |
| | MFAP4 | | | | | |
| | CNTFR | | | | | |

Each of these biomarker profiles may be used to identify a cancer that is SVV-S.

The genes selected for inclusion in a CLIA assay (an assay conducted under the Clinical Laboratory Improvement Amendments of 1988 (CLIA)) for screening patients for inclusion in a clinical trial are all up-regulated in SVV-S cells and tend to have very low expression in SVV-R cells.

### Example 2—SVV Biomarker Gene Set for Pre-Screening Patients

Based on the in-silico testing and the testing screening of 265 sarcoma samples, the following gene set was identified for screening patients for cancers that are susceptible to treatment with SVV:

TABLE 7
Gene set for pre-screening patients

EFS
MFAP4
NTN3
SOX11
NEFH
CNTFR
JPH4
ANTXR1

These eight markers provide the following parameters for the assay:

TABLE 8

| | | | Positive | Negative |
|---|---|---|---|---|
| Predication | | | Predictive | Predictive |
| Accuracy | Sensitivity | Specificity | Value | Value |
| 86 | 92 | 74 | 88 | 81 |

The SVV-sensitivity biomarker panel disclosed in Table 7 was tested in 81 SCLC patients, 15 NEC patient, and 69 PDX models. SCLC-sensitive/SCLC=60% (49/81) patients are projected to be SVV-Sensitive. NEC-sensitive/NEC=62% (43/69) NEC/SCLC PDX models are projected to be SVV-Sensitive. Based on this testing, it can be concluded that the SVV-Biomarker Panel should allow selection of patients who are likely to respond to SVV-001 or SVV derivative.

### Example 3: Using the SVV Biomarker Panel

265 sarcoma patient samples from the TCGA database were analyzed (see above). The analyzed samples are identified in Table 9 below:

TABLE 9
List of analyzed sarcoma samples from TCGA

| ONCOTREE_{—} | | |
|---|---|---|
| CODE | Detail | Count |
| CONTROL | Normal | 2 |
| DDLS | Dedifferentiated Liposarcoma | 57 |
| DES | Desmoid/Aggressive Fibromatosis | 2 |
| LMS | Leiomyosarcoma | 104 |
| MFH | Undifferentiated Pleomorphic Sarcoma/ | 49 |
| | Malignant Fibrous Histiocytoma/High | |
| | Grade Spindle Cell Sarcoma | |
| MFS | Myxofibrosarcoma | 25 |
| MPNST | Malignant Peripheral Nerve Sheath Tumor | 9 |
| PLLS | Pleomorphic Liposarcoma | 2 |
| SARCNOS | Soft Tissue Sarcoma | 1 |
| SOFT_TISSUE | Soft Tissue | 4 |
| SYNS | Synovial Sarcoma | 10 |

In all of these sarcoma samples, ANTRX1/TEM8 expression was observed to be high. The following probabilities of SVV-sensitivity of these 265 sarcoma tumors were determined:

TABLE 10
Probability of SVV-sensitivity of sarcoma tumors

| Probability | # positive tumors |
|---|---|
| Equal to 1 | 2 |
| Greater than 0.75 and less than 1 | 249 (94%) |
| Between 0.5-0.75 | 9 |
| Less than 0.5 | 5 |

94.7% were predicted as SVV-sensitive (251 out of 265 have a probability of SVV-sensitivity >0.75).

10361 TCGA patient samples used to test the 8-gene biomarker panel were predicted to be SVV sensitive.

### Example 4. Bioinformatics Analyses-Screening TCGA Samples with SVV Biomarker Gene Panel

The 8-Gene Panel: EFS, MFAP4, NTN3, SOX11, NEFH, CNTFR, JPH4, ANTXR1 was used against RNAseq data from TCGA tumors (n=37 tumor types)—no SCLC or NEC available. The total TCGA samples were 9651 tumor samples. 4619 of these were predicted as sensitive, and 5 tumor types showed high probability (>82%) of being SVV-Sensitive using a 75% confidence limit. These tumor types were: LGG—lower grade glioma; GBM—glioblastoma multiforme; PCPG—phaeochromocytoma and para-ganglioma; PRAD—prostate adenocarcinoma; UCS—uterine carcinosarcoma.

The list of all the tumor types tested is presented in Table 11. The results are shown in Table 12. A detailed analysis of tumors with high probability of SVV sensitivity is shown in Table 13.

Six out of 37 tumor types showed to be highly sensitive to SVV with the probability of SVV-Sensitivity of >82%:
LGG: most common CNS tumors among children and adolescents; incidence rate of 2 to 3 per 100.000 children. Up to 20% of patients with NF1 develop a low-grade glioma within the first 20 years of their life, mostly in the area of the optic pathway or in the lower brain stem. Up to 15% of patients with tuberous sclerosis are diagnosed with subependymal giant cell astrocytoma, a certain subtype of low-grade glioma. More than 90% of all LGG patients survive their disease long-term.

GBM: median survival approx. 12 months.

PCPG: rare NET; catecholamine-producing tumors that arise from adrenal medulla or from extra-adrenal ganglial sympathetic/parasympathetic chains. Patients with metastatic PCPG have a 5-year survival rate ranging from 40-77%.

UCS: rare aggressive high-grade endometrial cancer (approx. 5% of endometrial cancer and increasing). The 5-year survival rate is approx. 50%.

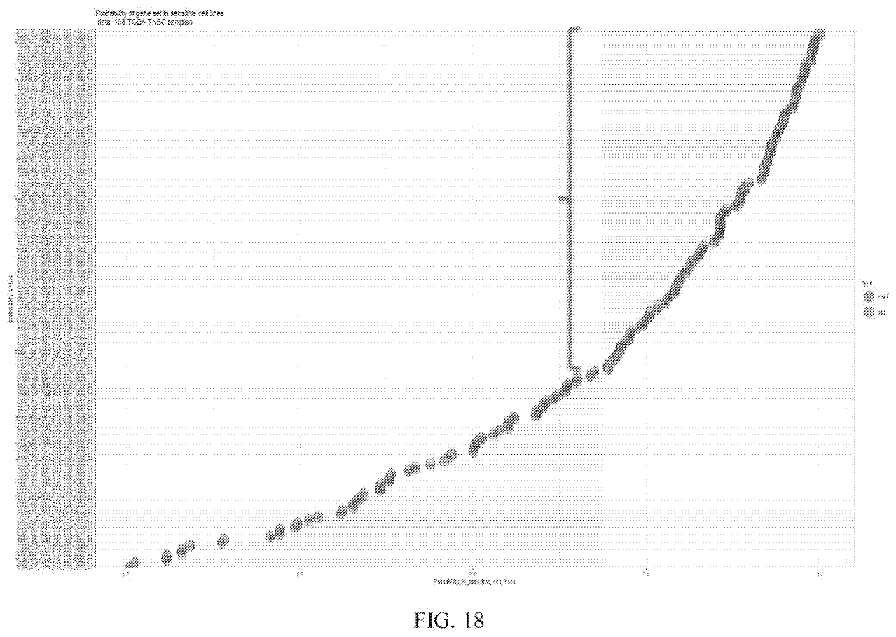

Triple negative breast cancer (TNBC): 10-15% of all breast cancers. Overall 5%-year survival rate is 77%, but patients with metastatic disease have only 12% survival rate at 5 years, with median survival of 13 months. Using 75% probability of SVV-Sensitive TNBC from BRCA, 103/163 samples are predicted to be SVV-Sensitive (63%). These samples were tested for the status for HER2 expression by two methods: IHC and FISH. FISH negative samples were 65 samples, IHC negative samples were 116 samples. Total: 163 (18 common results were labelled in IHC). The results are shown in FIG. 18.

PRAD: Both ERG+ and ETV1+prostate cancers are likely to be sensitive to SVV. ERG+ cancer comprise 46% of all prostate cancers (100,000 pts/year) whereas ETV1+comprise about 4% of PCa. ERG+ and ETV1+PCa have reduced OS.

TABLE 11
List of all the tumor types tested for sensitivity to SVV using
the 8 gene biomarker gene panel bioinformatics analysis.

| | |
|---|---|
| LAML | Acute Myeloid Leukemia |
| ACC | Adrenocortical carcinoma |
| BLCA | Bladder Urothelial Carcinoma |
| LGG | Brain Lower Grade Glioma |
| BRCA | Breast invasive carcinoma |
| CESC | Cervical squamous cell carcinoma and endocervical |
| | adenocarcinoma |
| CHOL | Cholangiocarcinoma |
| LCML | Chronic Myelogenous Leukemia |
| COAD | Colon adenocarcinoma |
| CNTL | Controls |
| ESCA | Esophageal carcinoma |
| FPPP | FFPE Pilot Phase II |
| GBM | Glioblastoma multiforme |
| HNSC | Head and Neck squamous cell carcinoma |
| KICH | Kidney Chromophobe |
| KIRC | Kidney renal clear cell carcinoma |
| KIRP | Kidney renal papillary cell carcinoma |
| LIHC | Liver hepatocellular carcinoma |
| LUAD | Lung adenocarcinoma |
| LUSC | Lung squamous cell carcinoma |
| DLBC | Lymphoid Neoplasm Diffuse Large B-cell Lymphoma |
| MESO | Mesothelioma |
| MISC | Miscellaneous |
| OV | Ovarian serous cystadenocarcinoma |
| PAAD | Pancreatic adenocarcinoma |
| PCPG | Pheochromocytoma and Paraganglioma |
| PRAD | Prostate adenocarcinoma |
| READ | Rectum adenocarcinoma |
| SARC | Sarcoma |
| SKCM | Skin Cutaneous Melanoma |
| STAD | Stomach adenocarcinoma |
| TGCT | Testicular Germ Cell Tumors |
| THYM | Thymoma |
| THCA | Thyroid carcinoma |
| UCS | Uterine Carcinosarcoma |
| UCEC | Uterine Corpus Endometrial Carcinoma |
| UVM | Uveal Melanoma |

TABLE 12
Tumor types ranked by their sensitivity to SVV based on
the 8 gene biomarker gene panel bioinformatics analysis.

| Tumor_type | Sensitive1(p > 0.75) | Sensitive2(p > 0.5) | Total | Sensitive1/Total | Sensitive2/Total |
|---|---|---|---|---|---|
| LGG | 530 | 530 | 530 | 1 | 1 |
| GBM | 164 | 165 | 166 | 0.987951807 | 0.993975904 |
| PCPG | 174 | 181 | 184 | 0.945652174 | 0.983695652 |
| PRAD | 417 | 462 | 498 | 0.837349398 | 0.927710843 |
| UCS | 47 | 51 | 57 | 0.824561404 | 0.894736842 |
| THYM | 60 | 85 | 120 | 0.5 | 0.708333333 |
| BRCA | 509 | 802 | 1100 | 0.462727273 | 0.729090909 |
| TGCT | 70 | 104 | 156 | 0.448717949 | 0.666666667 |
| OV | 122 | 169 | 307 | 0.397394137 | 0.550488599 |
| CESC | 102 | 157 | 306 | 0.333333333 | 0.513071895 |
| MESO | 23 | 54 | 87 | 0.264367816 | 0.620689655 |
| UVM | 21 | 40 | 80 | 0.2625 | 0.5 |
| PAAD | 46 | 103 | 179 | 0.25698324 | 0.575418994 |
| LUSC | 121 | 297 | 501 | 0.241516966 | 0.592814371 |
| BLCA | 69 | 200 | 408 | 0.169117647 | 0.490196078 |
| HNSC | 85 | 216 | 522 | 0.162835249 | 0.413793103 |
| ESCA | 27 | 69 | 185 | 0.145945946 | 0.372972973 |
| SKCM | 67 | 128 | 472 | 0.141949153 | 0.271186441 |
| UCEC | 52 | 95 | 370 | 0.140540541 | 0.256756757 |
| STAD | 58 | 130 | 415 | 0.139759036 | 0.313253012 |
| LUAD | 69 | 205 | 517 | 0.133462282 | 0.396518375 |
| ACC | 5 | 8 | 79 | 0.063291139 | 0.101265823 |
| KIRC | 15 | 70 | 534 | 0.028089888 | 0.131086142 |
| LIHC | 10 | 27 | 373 | 0.026809651 | 0.072386059 |
| THCA | 13 | 55 | 509 | 0.025540275 | 0.10805501 |
| COAD | 7 | 23 | 287 | 0.024390244 | 0.080139373 |
| READ | 2 | 6 | 95 | 0.021052632 | 0.063157895 |
| KICH | 1 | 1 | 66 | 0.015151515 | 0.015151515 |
| KIRP | 2 | 8 | 291 | 0.006872852 | 0.027491409 |
| CHOL | 0 | 2 | 36 | 0 | 0.055555556 |
| DLBC | 0 | 0 | 48 | 0 | 0 |
| LAML | 0 | 0 | 173 | 0 | 0 |

TABLE 13
Tumors with high probability of sensitivity to SVV based on
the 8 gene biomarker gene panel bioinformatics analysis.

| | | #Sensitive | Total # | Freq of SVV-Sensitive/ | |
|---|---|---|---|---|---|
| Tumor | Tumor_type | (p > 0.75) | tumors | Total at 75% cutoff level | |
| Lower Grade Glioma | LGG | 530 | 530 | 1 | |
| Glioblastoma Multiforme | GBM | 164 | 166 | 0.987951807 | |
| Paraganglioma & | PCPG | 174 | 184 | 0.945652174 | |
| Pheochromocytoma | | | | | |
| Sarcoma | SARC | 226 | 265 | 0.859 | |
| Prostate Adenocarcinoma | PRAD | 417 | 498 | 0.837349398 | |
| Uterine Carcinosarcoma | UCS | 47 | 57 | 0.824561404 | |
| Triple Negative Breast ca | TNBC | 103 | 162 | 0.64 | subset of BRCA |
| Thyroid Papillary Carcinoma | THYM | 60 | 120 | 0.5 | |
| Breast Ductal Carcinoma | BRCA | 509 | 1100 | 0.462727273 | |
| Testicular Germ Cell Cancer | TGCT | 70 | 156 | 0.448717949 | |
| Small Cell Lung Cancer | SCLC | 35 | 81 | 0.43 | 0.68 using 50% |
| | | | | | cutoff. Cologne |
| | | | | | SCLC dataset |

### Example 5. Developing the Biomarker Assay

To develop a patient biomarker assay using the 8-gene biomarker panel, primers for qRT-PCR and SVV-S and SVV-R cell lines were used. The RNA was isolated from SVV-S and -R lines and tested using biomarker primers. Analytical robustness was demonstrated by precision, sensitivity, selectivity, and specificity to develop a fit-for-purpose CLIA assay.

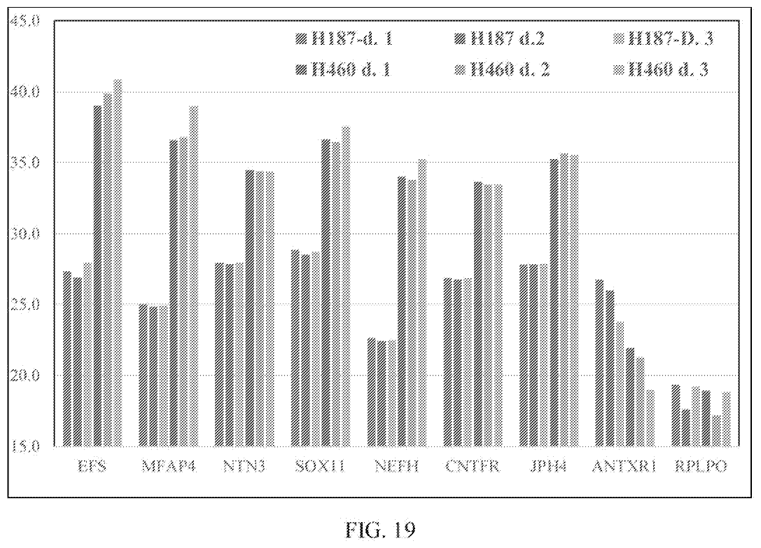

An exemplary assay was developed and tested on two cell lines: the NCI-H187 cell line (an SVV permissive cell line) and the H460 cell line (an SVV non-permissive cell line). The gene panel used was the 8-gene panel (Table 7) plus RPLPO. Assay reproducibility and variability was determined from average of Ct values from Day 1, Day 2, and Day 3. Each sample was tested in triplicate in each of the assay on Day 1, Day 2, and Day 3. The results are shown in FIG. 19 and in Table 14.

The assay was used for comparing the expression of the 8-gene panel (Table 7) plus RPLPO in SVV sensitive and SVV resistant cell lines. RNA was isolated from 20 cell lines and they were screened using the 8-gene panel. RPLPO was used as a housekeeping control.

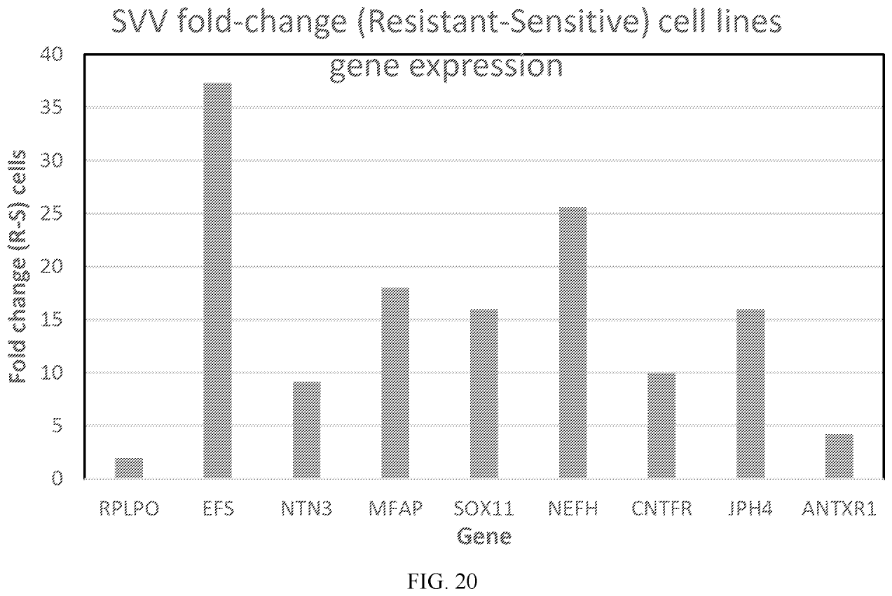

The RT-qPCR was used to evaluate expression of 8 genes. The assay used specific primers for each target gene and demonstrated that the 8-gene panel can select between SVV-001 Sensitive and Resistant cells as indicated in FIG. 20. Both RPLPO and TEM8 (ANTXR1) showed similar results in SVV-S versus SVV-R cell lines whereas the other biomarker genes showed significant changes between SVV-S and SVV-R cell lines.

TABLE 14
Assay reproducibility for the 8-gene panel plus RPLPO qRT-
PCR assay tested in NCI-H187 and H460 cells. The data represent
average of Ct values from Day 1, Day 2, and Day 3.

| Assay Reproducibility | |
|---|---|
| NCI-H187 | H460 |

| | Avg. | % CV | Avg. | % CV |
|---|---|---|---|---|
| EFS | 27.4 | 1.6 | 39.9 | 1.9 |
| MFAP4 | 24.9 | 0.3 | 37.4 | 2.9 |
| NTN3 | 27.9 | 0.1 | 34.4 | 0.2 |
| SOX11 | 28.7 | 0.5 | 36.9 | 1.3 |
| NEFH | 22.5 | 0.4 | 34.4 | 1.8 |
| CNTFR | 26.8 | 0.2 | 33.5 | 0.3 |
| JPH4 | 27.8 | 0.1 | 35.5 | 0.4 |
| ANTXR1 | 25.5 | 5.0 | 20.7 | 6.1 |
| RPLPO | 18.7 | 4.3 | 18.3 | 4.3 |

The 8-gene panel plus RPLPO qRT-PCR was conducted using the following materials and methods. Standard RNA input was 200 ng per 20 μL reaction, (or 150 ng per 40 μL reaction in PTI protocol). For standard reaction parameters per well, the RNA input was 200 ng in 13.2 μL, Master Mix (MM) volume=6.8 μL, and total reaction volume was 20 μL. For large assays, the reaction volume per well could be doubled. The reverse transcription (RT) master mix (MM) was prepared using the components and volumes specified in Table 15.

TABLE 15
RT master mix.

| | Standard Vol | Doubled Vol |
|---|---|---|
| Component | (μL) (n = 1) | (n = 1) |
| 10x RT Buffer | 2 | 4 |
| 25x dNTPs | 0.8 | 1.6 |
| 10x random primers | 2 | 4 |
| RNase Inhibitor | 1 | 2 |
| RT Enzyme | 1 | 2 |
| Total | 6.8 | 13.6 |

The cDNA synthesis conditions were as indicated in Table 16.

TABLE 16
cDNA synthesis conditions.

| Temp (°C) | Time | |
|---|---|---|
| 25 | 10 | min |
| 37 | 120 | min |
| 85 | 5 | min |

| | |
|---|---|
| 4 | forever |

The qPCR was conducted using 3 μL cDNA input and qPCR MM. The qPCR MM was prepared using the components and volumes specified in Table 17.

TABLE 17
qPCR master mix.

| Component | Vol (μL) (n = 1) |
|---|---|
| Taqman ® Fast Adv MM (2x) | 10 |
| Probes | 1 ea. |
| H2O | to 17 μL |
| Total | 17 |

The MM was prepared with 20% extra volume. For standard reaction parameters per well, 17 μL of MM was added to all wells, 3 μL cDNA was added to all wells, and qPCR was run according to the amplification conditions specified in Table 18.

TABLE 18
qPCR amplification conditions.

| Temp (°C) | Time | | Repeats |
|---|---|---|---|
| 50 | 2 | min | |
| 95 | 20 | sec | |
| 95 | 3 | sec | x40 |
| 60 | 30 | sec | |

The details of the cDNA RT kit are found in Applied Biosystems High Capacity cDNA Reverse Transcription Kit User Guide, publication #MAN0017977, revision A.0 (Jul. 16, 2018).

The details of the qPCR master mix are found in Applied Biosystems TaqMan Fast Advanced Master Mix User Guide, publication #4444605, revision D.

The Ct values obtained from the qRT-PCR amplification of the 8 genes of the 8-gene panel and RPLPO were analyzed as follows. The mean Ct value for RPLPO was subtracted from the mean Ct value of each gene per cell line examined to obtain a delta Ct value (ΔCt) per gene per cell line. The fold change for each gene over RPLPO per cell line was calculated from the following formula: Fold Change=2\hat{ }−ΔCt.

The obtained fold change values per gene were then compared between different cell lines.

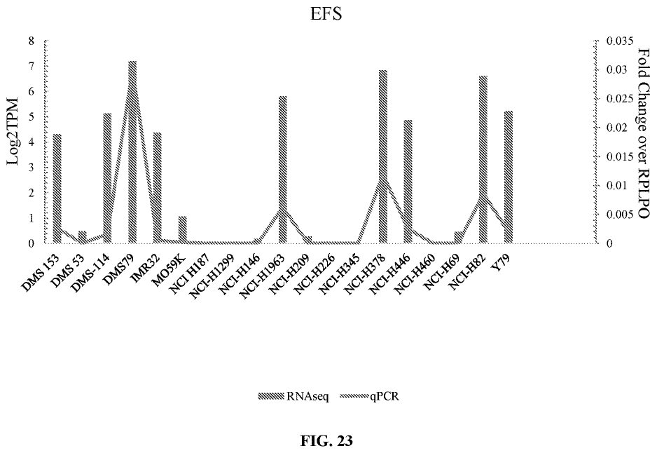

The qRT-PCR assay was performed on 19 different lung cancer cells. The assay produced fold change values for all the 8 genes in the 8-gene panel, as presented in Table 19. The fold change values for the EFS gene obtained from the 19 lung cancer cell lines is presented in FIG. 23. An increase in fold change over RPLPO indicated that the cell line was sensitive.

TABLE 19
Fold change values for the 8 genes of the 8-gene panel obtained from 19 lung cancer cell lines.

| Cell | | | | | | | | |
|---|---|---|---|---|---|---|---|---|
| line | Fold_ | Fold_ | Fold_ | Fold_ | Fold_ | Fold_ | Fold_ | Fold_ |
| name | EFS | MFAP4 | NTN3 | SOX11 | NEFH | CNTFR | JPH4 | ANTXR1 |
| NCI- | 0.00000 | 0.00000 | 0.00003 | 0.00000 | 0.00002 | 0.00007 | 0.00000 | 0.24335 |
| H460 | | | | | | | | |
| NCI- | 0.00000 | 0.00003 | 0.00012 | 0.00000 | 0.00000 | 0.00003 | 0.00000 | 0.08654 |
| H226 | | | | | | | | |
| DMS79 | 0.03004 | 0.00102 | 0.01564 | 0.02628 | 0.00474 | 0.06200 | 0.00048 | 0.03257 |
| NCI | 0.00000 | 0.00000 | 0.01134 | 0.02459 | 0.00047 | 0.00039 | 0.00001 | 0.00961 |
| H187 | | | | | | | | |
| IMR32 | 0.00055 | 0.11642 | 0.00438 | 0.00654 | 0.00135 | 0.02262 | 0.00111 | 0.42141 |
| NCI- | 0.01189 | 0.00365 | 0.01267 | 0.01884 | 0.00080 | 0.01718 | 0.00093 | 0.02087 |
| H378 | | | | | | | | |
| NCI- | 0.00001 | 0.00810 | 0.00491 | 0.00213 | 0.00017 | 0.00087 | 0.00010 | 0.00664 |
| H209 | | | | | | | | |
| NCI- | 0.00000 | 0.00000 | 0.00009 | 0.00000 | 0.00060 | 0.00002 | 0.00000 | 0.01265 |
| H1299 | | | | | | | | |
| NCI- | 0.00254 | 0.22785 | 0.01064 | 0.00063 | 0.00000 | 0.03943 | 0.00195 | 0.01799 |
| H446 | | | | | | | | |
| Y79 | 0.00205 | 0.04939 | 0.01901 | 0.00172 | 0.00394 | 0.02536 | 0.00002 | 0.01207 |
| NCI- | 0.00856 | 0.00874 | 0.02103 | 0.00165 | 0.00713 | 0.02258 | 0.00024 | 0.01390 |
| H82 | | | | | | | | |
| NCI- | 0.00627 | 0.06347 | 0.00567 | 0.00955 | 0.00118 | 0.04781 | 0.00347 | 0.03663 |
| H1963 | | | | | | | | |
| DMS | 0.00266 | 0.00005 | 0.01269 | 0.00833 | 0.00045 | 0.00010 | 0.00031 | 0.01039 |
| 153 | | | | | | | | |
| MO59K | 0.00022 | 0.00021 | 0.00036 | 0.00000 | 0.00000 | 0.00010 | 0.00000 | 0.08028 |
| NCI- | 0.00001 | 0.03669 | 0.00257 | 0.00051 | 0.00002 | 0.00543 | 0.00003 | 0.00100 |
| H69 | | | | | | | | |
| DMS | 0.00005 | 0.00000 | 0.00065 | 0.00155 | 0.00003 | 0.00309 | 0.00011 | 0.00699 |
| 53 | | | | | | | | |
| DMS- | 0.00163 | 0.00000 | 0.00018 | 0.00000 | 0.00039 | 0.01567 | 0.00005 | 0.00046 |
| 114 | | | | | | | | |
| NCI- | 0.00001 | 0.00001 | 0.00231 | 0.00314 | 0.00019 | 0.00321 | 0.00085 | 0.00020 |
| H146 | | | | | | | | |
| NCI- | 0.00000 | 0.00010 | 0.01746 | 0.04001 | 0.00004 | 0.00063 | 0.00039 | 0.01401 |
| H345 | | | | | | | | |

The protocol for determining if a cell line or patient was sensitive to SVV using RNAseq was as follows:
1. Using the RNAseq data, determine the gene expression (Transcripts Per Million) of the following genes: EFS, NTN3, MFAP4, SOX11, NEFH, CNTFR, JPH4 and ANTXR1.
2. Multiply the gene expression of each gene by a determined weight for each gene (array multiplication).
3. The result of this array multiplication represents a score. If the score is higher than 81, then the cell line or patient will be assigned as sensitive.

More specifically, the protocol used to determine if a cell line/patient was sensitive to SVV, was as follows:
1. Using the RNAseq data, the gene expression (Transcripts Per Million (TPM)) of the following genes: EFS, NTN3, MFAP4, SOX11, NEFH, CNTFR, JPH4 and ANTXR1 was determined.
2. The log₂(TPM+1) number was calculated.
3. The calculated number from step 2 was multiplied by a determined weight for each gene (array multiplication). The weight of each gene was determined with the bioinformatic analysis, and demonstrates which gene has more relevance at the moment of calculating the score (the higher the weight, the more important the gene is within the score). The weights for each gene are presented in Table 20.

TABLE 20
Weights for the genes of the 8-gene biomarker panel.

| Gene | Weight |
|---|---|
| EFS | 5.894347 |
| NTN3 | 4.48852 |
| MFAP4 | 4.926548 |
| SOX11 | 4.575808 |
| NEFH | 4.440061 |
| CNTFR | 4.398047 |
| JPH4 | 3.718045 |
| ANTXR1 | 2.675375 |

4. The result of the array multiplication obtained in step 3 represented a score. If the score was higher than 81.2733, then the cell line or patient was assigned as sensitive.

Nineteen lung cancer cell lines were screened with RNAseq and with the qRT-PCR assay for the 8-gene panel to demonstrate that both assays could detect SVV-resistant and SVV-sensitive cells at similar accuracy. Strong correlations between (R:0.90-0.98) qPCR results and RNAseq results was seen in EFS, MFAP4, SOX11, NEFH, CNTFR, and JPH4 genes. High correlations between qPCR results and RNAseq results was detected NTN3 and ANTXR1 genes (R: 0.70-0.90). An example is shown in FIG. 23 for the EFS gene. FIG. 23 demonstrates the strong correlation between the qPCR results and RNAseq results obtained for this gene across the 19 cell lines examined.

The above two protocols were used to predict and then to experimentally validate the sensitivity or resistance of 19 different cell lines to SVV. The results are shown in Table 21.

TABLE 21
qRT-PCR assay validation using19 lung cancer cell lines.

| Cell line name | Prediction | Experimental |
|---|---|---|
| DMS79 | Sensitive | Resistant |
| DMS114 | Sensitive | Resistant |
| NCIH226 | Resistant | Resistant |
| DMS53 | Resistant | Resistant |
| NCIH69 | Resistant | Resistant |
| NCIH146 | Resistant | Resistant |
| NCIH460 | Resistant | Resistant |
| NCIH345 | Resistant | Resistant |
| NCIH209 | Sensitive | Sensitive |
| NCIH82 | Sensitive | Sensitive |
| NCIH1299 | Resistant | Sensitive |
| DMS153 | Sensitive | Sensitive |
| M059K | Resistant | Sensitive |
| NCIH1963 | Sensitive | Sensitive |
| IMR32 | Sensitive | Sensitive |
| NCIH446 | Sensitive | Sensitive |
| Y79 | Sensitive | Sensitive |
| NCIH378 | Sensitive | Sensitive |
| NCIH187 | Sensitive | Sensitive |

Based on this experimental data, it was established that the qRT-PCR assay predicted the sensitivity or resistance of a cell line to SVV at high accuracy and sensitivity. The results are show in Table 22.

TABLE 22
Accuracy and sensitivity of the
8-gene panel plus RPLPO qRT-PCR.

| | Experimental | Theoretical |
|---|---|---|
| Accuracy | 0.789 | 0.859 |
| Sensitivity | 0.818 | 0.916 |
| PPV | 0.818 | 0.880 |
| NPV | 0.750 | 0.808 |

TABLE 23
Gene names and accession numbers for
several of the disclosed biomarkers.

| Gene Name | Gene ID | Accession # |
|---|---|---|
| ACCN4 | 55515 | NM_018674 |
| ALPL | 249 | NM_000478.6 |
| ANTXR1 | 84168 | NM_032208.2 |
| ATP1B2 | 482 | NM_001678.5 |
| CAMKV | 79012 | NM_024046.5 |
| CD248 | 57124 | NM_020404.3 |
| CNTFR | 1271 | NM_147164.3 |
| COL25A1 | 84570 | NM_198721.4 |
| CUX2 | 23316 | NM_015267.4 |
| EFS | 10278 | NM_005864.4 |
| ELN | 2006 | NM_000501.4 |
| HIF3A | 64344 | NM_152795.4 |
| HPCA | 3208 | NM_002143.3 |
| ISLR | 3671 | NM_005545.4 |
| JPH4 | 84502 | NM_032452.3 |
| LPPR4 | 9890 | NM_014839.5 |
| MFAP4 | 4239 | NM_001198695.2 |
| NEFH | 4744 | NM_021076.4 |
| NEUROD1 | 4760 | NM_002500.5 |
| NHLH1 | 4807 | NM_005598.4 |
| NTN3 | 4917 | NM_006181.3 |
| SEMA5B | 54437 | NM_001031702.4 |
| SOX11 | 6664 | NM_003108.4 |
| VAT1L | 57687 | NM_020927.3 |

TABLE 24
Nucleic acid sequence for SVV-001.

| Name | Nucleic Acid Sequence | |
|---|---|---|
| SVV-001 | 1 | tttgaaatgg ggggctgggc cctgatgccc agtccttcct ttccccttcc ggggggttaa |
| (SEQ ID NO: 1) | 61 | ccggctgtgt ttgctagagg cacagagggg caacatccaa cctgcttttg cggggaacgg |
| | 121 | tgcggctccg attcctgcgt cgccaaaggt gttagcgcac ccaaacggcg cacctaccaa |
| | 181 | tgttattggt gtggtctgcg agttctagcc tactcgtttc tcccccgacc attcactcac |
| | 241 | ccacgaaaag tgtgttgtaa ccataagatt taacccccgc acgggatgtg cgataaccgt |
| | 301 | aagactggct caagcgcgga aagcgctgta accacatgct gttagtccct ttatggctgc |
| | 361 | aagatggcta cccacctcgg atcactgaac tggagctcga ccctccttag taagggaacc |
| | 421 | gagaggcctt cgtgcaacaa gctccgacac agagtccacg tgactgctac caccatgagt |
| | 481 | acatggttct cccctctcga cccaggactt ctttttgaat atccacggct cgatccagag |
| | 541 | ggtggggcat gacccctagc atagcgagct acagcgggaa ctgtagctag gccttagcgt |
| | 601 | gccttggata ctgcctgata gggcgacggc ctagtcgtgt cggttctata ggtagcacat |
| | 661 | acaaatatgc agaactctca tttttctttc gatacagcct ctggcacctt tgaagatgta |
| | 721 | accggaacaa aagtcaagat cgttgaatac cccagatcgg tgaacaatgg tgtttacgat |
| | 781 | tcgtctactc atttggagat actgaaccta cagggtgaaa ttgaaatttt aaggtctttc |
| | 841 | aatgaatacc aaattcgcgc cgccaaacaa caactcggac tggacatcgt gtacgaacta |
| | 901 | cagggtaatg ttcagacaac gtcaaagaat gattttgatt cccgtggcaa taatggtaac |
| | 961 | atgaccttca attactacgc aaacacttat cagaattcag tagacttctc gacctcctcg |
| | 1021 | tcggcgtcag gcgccggacc cgggaactcc cggggcggat tagcgggtct cctcacaaat |
| | 1081 | ttcagtggaa tcttgaaccc tcttggctac ctcaaagatc acaacaccga agaaatggaa |
| | 1141 | aactctgctg atcgagtcac aacgcaaacg gcgggcaaca ctgccataaa cacgcaatca |
| | 1201 | tcattgggtg tgttgtgtgc ctacgttgaa gacccgacca aatctgatcc tccgtccagc |
| | 1261 | agcacagatc aacccaccac cactttcact gccatcgaca ggtggtacac tggacgtctc |
| | 1321 | aattcttgga caaaagctgt aaaaaccttc tcttttcagg ccgtcccgct tcccggggcc |
| | 1381 | tttctgtcta ggcagggagg cctcaacgga ggggccttca cagctaccct acatagacac |
| | 1441 | tttttgatga agtgcgggtg gcaggtgcag gtccaatgta atttgacaca attccaccaa |
| | 1501 | ggcgctcttc ttgttgccat ggttcctgaa accacccttg atgtcaagcc cgacggtaag |
| | 1561 | gcaaagagct tacaggagct gaatgaagaa cagtgggtgg aaatgtctga cgattaccgg |
| | 1621 | accgggaaaa acatgccttt tcagtctctt ggcacatact atcggccccc taactggact |
| | 1681 | tggggtccca atttcatcaa cccctatcaa gtaacggttt tcccacacca aattctgaac |
| | 1741 | gcgagaacct ctacctcggt agacataaac gtcccataca tcggggagac ccccacgcaa |
| | 1801 | tcctcagaga cacagaactc ctggaccctc ctcgttatgg tgctcgttcc cctagactat |
| | 1861 | aaggaaggag ccacaactga cccagaaatt acattttctg taaggcctac aagtccctac |
| | 1921 | ttcaatgggc ttcgcaaccg ctacacggcc gggacggacg aagaacaggg gcccattcct |
| | 1981 | acggcaccca gagaaaattc gcttatgttt ctctcaaccc tccctgacga cactgtccct |
| | 2041 | gcttacggga atgtgcgtac ccctcctgtc aattacctcc ctggtgaaat aaccgacctt |
| | 2101 | ttgcaactgg cccgcatacc cactctcatg gcatttgagc gggtgcctga acccgtgcct |
| | 2161 | gcctcagaca catatgtgcc ctacgttgcc gttcccaccc agttcgatga caggcctctc |
| | 2221 | atctccttcc cgatcaccct ttcagatccc gtctatcaga acaccctggt tggcgccatc |
| | 2281 | agttcaaatt tcgccaatta ccgtgggtgt atccaaatca ctctgacatt ttgtggaccc |
| | 2341 | atgatggcga gagggaaatt cctgctctcg tattctcccc caaatggaac gcaaccacag |
| | 2401 | actctttccg aagctatgca gtgcacatac tctatttggg acataggctt gaactctagt |
| | 2461 | tggaccttcg tcgtccccta catctcgccc agtgactacc gtgaaactcg agccattacc |
| | 2521 | aactcggttt actccgctga tggttggttt agcctgcaca agttgaccaa aattactcta |
| | 2581 | ccacctgact gtccgcaaag tccctgcatt ctctttttcg cttctgctgg tgaggattac |
| | 2641 | actctccgtc tccccgttga ttgtaatcct tcctatgtgt tccactccac cgacaacgcc |
| | 2701 | gagaccgggg ttattgaggc gggtaacact gacaccgatt tctctggtga actggcggct |
| | 2761 | cctggctcta accacactaa tgtcaagttc ctgtttgatc gatctcgatt attgaatgta |
| | 2821 | atcaaggtac tggagaagga cgccgttttc ccccgccctt tccctacaca agaaggtgcg |
| | 2881 | cagcaggatg atggttactt ttgtcttctg accccccgcc caacagtcgc ttcccgaccc |
| | 2941 | gccactcgtt tcggcctgta cgccaatccg tccggcagtg gtgttcttgc taacacttca |
| | 3001 | ctggacttca atttttatag cttggcctgt ttcacttact ttagatcgga ccttgaggtt |
| | 3061 | acggtggtct cactagagcc ggatctggaa tttgctgtag ggtggtttcc ttctggcagt |
| | 3121 | gaataccagg cttccagctt tgtctacgac cagctgcatg tgcccttcca ctttactggg |
| | 3181 | cgcactcccc gcgctttcgc tagcaagggt gggaaggtat ctttcgtgct cccttggaac |
| | 3241 | tctgtctcgt ctgtgctccc cgtgcgctgg gggggggctt ccaagctctc ttctgctacg |
| | 3301 | cggggtctac cggcgcatgc tgattggggg actatttacg cctttgtccc ccgtcctaat |
| | 3361 | gagaagaaaa gcaccgctgt aaaacacgtg gccgtgtaca ttcggtacaa gaacgcacgt |
| | 3421 | gcctggtgcc ccagcatgct tccctttcgc agctacaagc agaagatgct gatgcaatct |
| | 3481 | ggcgatatcg agaccaatcc tggtcctgct tctgacaacc caattttgga gtttcttgaa |
| | 3541 | gcagaaaatg atctagtcac tctggcctct ctctggaaga tggtgcactc tgttcaacag |
| | 3601 | acctggagaa agtatgtgaa gaacgatgat ttttggccca atttactcag cgagctagtg |
| | 3661 | ggggaaggct ctgtcgcctt ggccgccacg ctatccaacc aagcttcagt aaaggctctt |
| | 3721 | ttgggcctgc actttctctc tcgggggctc aattacactg acttttactc tttactgata |
| | 3781 | gagaaatgct ctagtttctt taccgtagaa ccacctcctc caccagctga aaacctgatg |
| | 3841 | accaagccct cagtgaagtc gaaattccga aaactgttta agatgcaagg acccatggac |
| | 3901 | aaagtcaaag actggaacca aatagctgcc ggcttgaaga attttcaatt tgttcgtgac |
| | 3961 | ctagtcaaag aggtggtcga ttggctgcag gcctggatca acaaagagaa agccagccct |
| | 4021 | gtcctccagt accagttgga gatgaagaag ctcgggcctg tggccttggc tcatgacgct |
| | 4081 | ttcatggctg gttccgggcc ccctcttagc gacgaccaga ttgaatacct ccagaacctc |
| | 4141 | aaatctcttg ccctaacact ggggaagact aatttggccc aaagtctcac cactatgatc |
| | 4201 | aatgccaaac aaagttcagc ccaacgagtt gaacccgttg tggtggtcct tagaggcaag |
| | 4261 | ccgggatgcg gcaagagctt ggcctctacg ttgattgccc aggctgtgtc caagcgcctc |
| | 4321 | tatggctccc aaagtgtata ttctcttccc ccagatccag atttcttcga tggatacaaa |
| | 4381 | ggacagttcg tgaccttgat ggatgatttg ggacaaaacc cggatggaca agatttctcc |
| | 4441 | accttttgtc agatggtgtc gaccgcccaa tttctcccca acatggcgga ccttgcagag |
| | 4501 | aaagggcgtc cctttacctc caatctcatc attgcaacta caaatctccc ccacttcagt |
| | 4561 | cctgtcacca ttgctgatcc ttctgcagtc tctcgccgta tcaactacga tctgactcta |
| | 4621 | gaagtatctg aggcctacaa gaaacacaca cggctgaatt ttgacttggc tttcaggcgc |
| | 4681 | acagacgccc cccccattta tccttttgct gcccatgtgc cctttgtgga cgtagctgtg |
| | 4741 | cgcttcaaaa atggtcacca gaattttaat ctcctagagt tggtcgattc catttgtaca |
| | 4801 | gacattcgag ccaagcaaca aggtgcccga aacatgcaga ctctggttct acagagcccc |
| | 4861 | aacgagaatg atgacacccc cgtcgacgag gcgttgggta gagttctctc ccccgctgcg |
| | 4921 | gtcgatgagg cgcttgtcga cctcactcca gaggccgacc cggttggccg tttggctatt |
| | 4981 | cttgccaagc taggtcttgc cctagctgcg gtcacccctg gtctgataat cttggcagtg |
| | 5041 | ggactctaca ggtacttctc tggctctgat gcagaccaag aagaaacaga aagtgaggga |
| | 5101 | tctgtcaagg cacccaggag cgaaaatgct tatgacggcc cgaagaaaaa ctctaagccc |
| | 5161 | cctggagcac tctctctcat ggaaatgcaa cagcccaacg tggacatggg ctttgaggct |
| | 5221 | gcggtcgcta agaaagtggt cgtccccatt accttcatgg ttcccaacag accttctggg |
| | 5281 | cttacacagt ccgctcttct ggtgaccggc cggaccttcc taatcaatga acatacatgg |
| | 5341 | tccaatccct cctggaccag cttcacaatc cgcggtgagg tacacactcg tgatgagccc |
| | 5401 | ttccaaacgg ttcatttcac tcaccacggt attcccacag atctgatgat ggtacgtctc |
| | 5461 | ggaccgggca attctttccc taacaatcta gacaagtttg gacttgacca gatgccggca |
| | 5521 | cgcaactccc gtgtggttgg cgtttcgtcc agttacggaa acttcttctt ctctggaaat |
| | 5581 | ttcctcggat ttgttgattc catcacctct gaacaaggaa cttacgcaag actctttagg |
| | 5641 | tacagggtga cgacctacaa aggatggtgc ggctcggccc tggtctgtga ggccggtggc |
| | 5701 | gtccgacgca tcattggcct gcattctgct ggcgccgccg gtatcggcgc cgggacctat |
| | 5761 | atctcaaaat taggactaat caaagccctg aaacacctcg gtgaaccttt ggccacaatg |
| | 5821 | caaggactga tgactgaatt agagcctgga atcaccgtac atgtaccccg gaaatccaaa |
| | 5881 | ttgagaaaga cgaccgcaca cgcggtgtac aaaccggagt ttgagcctgc tgtgttgtca |
| | 5941 | aaatttgatc ccagactgaa caaggatgtt gacttggatg aagtaatttg gtctaaacac |
| | 6001 | actgccaatg tcccttacca acctcctttg ttctacacat acatgtcaga gtacgctcat |
| | 6061 | cgagtcttct ccttcttggg gaaagacaat gacattctga ccgtcaaaga agcaattctg |
| | 6121 | ggcatccccg gactagaccc catggatccc cacacagctc cgggtctgcc ttacgccatc |
| | 6181 | aacggccttc gacgtactga tctcgtcgat tttgtgaacg gtacagtaga tgcggcgctg |
| | 6241 | gctgtacaaa tccagaaatt cttagacggt gactactctg accatgtctt ccaaactttt |
| | 6301 | ctgaaagatg agatcagacc ctcagagaaa gtccgagcgg gaaaaacccg cattgttgat |
| | 6361 | gtgccctccc tggcgcattg cattgtgggc agaatgttgc ttgggcgctt tgctgccaag |
| | 6421 | tttcaatccc atcctggctt tctcctcggc tctgctatcg ggtctgaccc tgatgttttc |
| | 6481 | tggaccgtca taggggctca actcgagggg agaaagaaca cgtatgacgt ggactacagt |
| | 6541 | gcctttgact cttcacacgg cactggctcc ttcgaggctc tcatctctca ctttttcacc |
| | 6601 | gtggacaatg gttttagccc tgcgctggga ccgtatctca gatccctggc tgtctcggtg |
| | 6661 | cacgcttacg gcgagcgtcg catcaagatt accggtggcc tcccctccgg ttgtgccgcg |
| | 6721 | accagcctgc tgaacacagt gctcaacaat gtgatcatca ggactgctct ggcattgact |
| | 6781 | tacaaggaat ttgaatatga catggttgat atcatcgcct acggtgacga ccttctggtt |
| | 6841 | ggcacggatt acgatctgga cttcaatgag gtggcacgac gcgctgccaa gttggggtat |
| | 6901 | aagatgactc ctgccaacaa gggttctgtc ttccctccga cttcctctct ttccgatgct |
| | 6961 | gtttttctaa agcgcaaatt cgtccaaaac aacgacggct tatacaaacc agttatggat |
| | 7021 | ttaaagaatt tggaagccat gctctcctac ttcaaaccag gaacactact cgagaagctg |
| | 7081 | caatctgttt ctatgttggc tcaacattct ggaaaagaag aatatgatag attgatgcac |
| | 7141 | cccttcgctg actacggtgc cgtaccgagt cacgagtacc tgcaggcaag atggagggcc |
| | 7201 | ttgttcgact gacccagata gcccaaggcg cttcggtgct gccggcgatt ctgggagaac |
| | 7261 | tcagtcggaa cagaaaaggg aaaaaaaaaa aaaaaaaaaa aaaaaaaaaa |

| | |
|---|---|
| SVV-001 | atgcagaactctcatttttctttcgatacagcctctggcacctttgaagatgtaaccggaacaaaagtca |
| nucleotides | agatcgttgaataccccagatcggtgaacaatggtgtttacgattcgtctactcatttggagatactgaa |
| 667-7212 | cctacagggtgaaattgaaattttaaggtctttcaatgaataccaaattcgcgccgccaaacaacaactc |
| NC 011349.1 | ggactggacatcgtgtacgaactacagggtaatgttcagacaacgtcaaagaatgattttgattcccgtg |
| (SEQ ID NO: | gcaataatggtaacatgaccttcaattactacgcaaacacttatcagaattcagtagacttctcgacctc |
| 2) | ctcgtcggcgtcaggcgccggacccgggaactcccggggcggattagcgggtctcctcacaaatttcagt |
| | ggaatcttgaaccctcttggctacctcaaagatcacaacaccgaagaaatggaaaactctgctgatcgag |
| | tcacaacgcaaacggcgggcaacactgccataaacacgcaatcatcattgggtgtgttgtgtgcctacgt |
| | tgaagacccgaccaaatctgatcctccgtccagcagcacagatcaacccaccaccactttcactgccatc |
| | gacaggtggtacactggacgtctcaattcttggacaaaagctgtaaaaaccttctcttttcaggccgtcc |
| | cgcttcccggggcctttctgtctaggcagggaggcctcaacggaggggccttcacagctaccctacatag |
| | acactttttgatgaagtgcgggtggcaggtgcaggtccaatgtaatttgacacaattccaccaaggcgct |
| | cttcttgttgccatggttcctgaaaccacccttgatgtcaagcccgacggtaaggcaaagagcttacagg |
| | agctgaatgaagaacagtgggtggaaatgtctgacgattaccggaccgggaaaaacatgccttttcagtc |
| | tcttggcacatactatcggccccctaactggacttggggtcccaatttcatcaacccctatcaagtaacg |
| | gttttcccacaccaaattctgaacgcgagaacctctacctcggtagacataaacgtcccatacatcgggg |
| | agacccccacgcaatcctcagagacacagaactcctggaccctcctcgttatggtgctcgttcccctaga |
| | ctataaggaaggagccacaactgacccagaaattacattttctgtaaggcctacaagtccctacttcaat |
| | gggcttcgcaaccgctacacggccgggacggacgaagaacaggggcccattcctacggcacccagagaaa |
| | attcgcttatgtttctctcaaccctccctgacgacactgtccctgcttacgggaatgtgcgtacccctcc |
| | tgtcaattacctccctggtgaaataaccgaccttttgcaactggcccgcatacccactctcatggcattt |
| | gagcgggtgcctgaacccgtgcctgcctcagacacatatgtgccctacgttgccgttcccacccagttcg |
| | atgacaggcctctcatctccttcccgatcaccctttcagatcccgtctatcagaacaccctggttggcgc |
| | catcagttcaaatttcgccaattaccgtgggtgtatccaaatcactctgacattttgtggacccatgatg |
| | gcgagagggaaattcctgctctcgtattctcccccaaatggaacgcaaccacagactctttccgaagcta |
| | tgcagtgcacatactctatttgggacataggcttgaactctagttggaccttcgtcgtcccctacatctc |
| | gcccagtgactaccgtgaaactcgagccattaccaactcggtttactccgctgatggttggtttagcctg |
| | cacaagttgaccaaaattactctaccacctgactgtccgcaaagtccctgcattctctttttcgcttctg |
| | ctggtgaggattacactctccgtctccccgttgattgtaatccttcctatgtgttccactccaccgacaa |
| | cgccgagaccggggttattgaggcgggtaacactgacaccgatttctctggtgaactggcggctcctggc |
| | tctaaccacactaatgtcaagttcctgtttgatcgatctcgattattgaatgtaatcaaggtactggaga |
| | aggacgccgttttcccccgccctttccctacacaagaaggtgcgcagcaggatgatggttacttttgtct |
| | tctgaccccccgcccaacagtcgcttcccgacccgccactcgtttcggcctgtacgccaatccgtccggc |
| | agtggtgttcttgctaacacttcactggacttcaatttttatagcttggcctgtttcacttactttagat |
| | cggaccttgaggttacggtggtctcactagagccggatctggaatttgctgtagggtggtttccttctgg |
| | cagtgaataccaggcttccagctttgtctacgaccagctgcatgtgcccttccactttactgggcgcact |
| | ccccgcgctttcgctagcaagggtgggaaggtatctttcgtgctcccttggaactctgtctcgtctgtgc |
| | tccccgtgcgctgggggggggcttccaagctctcttctgctacgcggggtctaccggcgcatgctgattg |
| | ggggactatttacgcctttgtcccccgtcctaatgagaagaaaagcaccgctgtaaaacacgtggccgtg |
| | tacattcggtacaagaacgcacgtgcctggtgccccagcatgcttccctttcgcagctacaagcagaaga |
| | tgctgatgcaatctggcgatatcgagaccaatcctggtcctgcttctgacaacccaattttggagtttct |
| | tgaagcagaaaatgatctagtcactctggcctctctctggaagatggtgcactctgttcaacagacctgg |
| | agaaagtatgtgaagaacgatgatttttggcccaatttactcagcgagctagtgggggaaggctctgtcg |
| | ccttggccgccacgctatccaaccaagcttcagtaaaggctcttttgggcctgcactttctctctcgggg |
| | gctcaattacactgacttttactctttactgatagagaaatgctctagtttctttaccgtagaaccacct |
| | cctccaccagctgaaaacctgatgaccaagccctcagtgaagtcgaaattccgaaaactgtttaagatgc |
| | aaggacccatggacaaagtcaaagactggaaccaaatagctgccggcttgaagaattttcaatttgttcg |
| | tgacctagtcaaagaggtggtcgattggctgcaggcctggatcaacaaagagaaagccagccctgtcctc |
| | cagtaccagttggagatgaagaagctcgggcctgtggccttggctcatgacgctttcatggctggttccg |
| | ggccccctcttagcgacgaccagattgaatacctccagaacctcaaatctcttgccctaacactggggaa |
| | gactaatttggcccaaagtctcaccactatgatcaatgccaaacaaagttcagcccaacgagttgaaccc |
| | gttgtggtggtccttagaggcaagccgggatgcggcaagagcttggcctctacgttgattgcccaggctg |
| | tgtccaagcgcctctatggctcccaaagtgtatattctcttcccccagatccagatttcttcgatggata |
| | caaaggacagttcgtgaccttgatggatgatttgggacaaaacccggatggacaagatttctccaccttt |
| | tgtcagatggtgtcgaccgcccaatttctccccaacatggcggaccttgcagagaaagggcgtcccttta |
| | cctccaatctcatcattgcaactacaaatctcccccacttcagtcctgtcaccattgctgatccttctgc |
| | agtctctcgccgtatcaactacgatctgactctagaagtatctgaggcctacaagaaacacacacggctg |
| | aattttgacttggctttcaggcgcacagacgccccccccatttatccttttgctgcccatgtgccctttg |
| | tggacgtagctgtgcgcttcaaaaatggtcaccagaattttaatctcctagagttggtcgattccatttg |
| | tacagacattcgagccaagcaacaaggtgcccgaaacatgcagactctggttctacagagccccaacgag |
| | aatgatgacacccccgtcgacgaggcgttgggtagagttctctcccccgctgcggtcgatgaggcgcttg |
| | tcgacctcactccagaggccgacccggttggccgtttggctattcttgccaagctaggtcttgccctagc |
| | tgcggtcacccctggtctgataatcttggcagtgggactctacaggtacttctctggctctgatgcagac |
| | caagaagaaacagaaagtgagggatctgtcaaggcacccaggagcgaaaatgcttatgacggcccgaaga |
| | aaaactctaagccccctggagcactctctctcatggaaatgcaacagcccaacgtggacatgggctttga |
| | ggctgcggtcgctaagaaagtggtcgtccccattaccttcatggttcccaacagaccttctgggcttaca |
| | cagtccgctcttctggtgaccggccggaccttcctaatcaatgaacatacatggtccaatccctcctgga |
| | ccagcttcacaatccgcggtgaggtacacactcgtgatgagcccttccaaacggttcatttcactcacca |
| | cggtattcccacagatctgatgatggtacgtctcggaccgggcaattctttccctaacaatctagacaag |
| | tttggacttgaccagatgccggcacgcaactcccgtgtggttggcgtttcgtccagttacggaaacttct |
| | tcttctctggaaatttcctcggatttgttgattccatcacctctgaacaaggaacttacgcaagactctt |
| | taggtacagggtgacgacctacaaaggatggtgcggctcggccctggtctgtgaggccggtggcgtccga |
| | cgcatcattggcctgcattctgctggcgccgccggtatcggcgccgggacctatatctcaaaattaggac |
| | taatcaaagccctgaaacacctcggtgaacctttggccacaatgcaaggactgatgactgaattagagcc |
| | tggaatcaccgtacatgtaccccggaaatccaaattgagaaagacgaccgcacacgcggtgtacaaaccg |
| | gagtttgagcctgctgtgttgtcaaaatttgatcccagactgaacaaggatgttgacttggatgaagtaa |
| | tttggtctaaacacactgccaatgtcccttaccaacctcctttgttctacacatacatgtcagagtacgc |
| | tcatcgagtcttctccttcttggggaaagacaatgacattctgaccgtcaaagaagcaattctgggcatc |
| | cccggactagaccccatggatccccacacagctccgggtctgccttacgccatcaacggccttcgacgta |
| | ctgatctcgtcgattttgtgaacggtacagtagatgcggcgctggctgtacaaatccagaaattcttaga |
| | cggtgactactctgaccatgtcttccaaacttttctgaaagatgagatcagaccctcagagaaagtccga |
| | gcgggaaaaacccgcattgttgatgtgccctccctggcgcattgcattgtgggcagaatgttgcttgggc |
| | gctttgctgccaagtttcaatcccatcctggctttctcctcggctctgctatcgggtctgaccctgatgt |
| | tttctggaccgtcataggggctcaactcgaggggagaaagaacacgtatgacgtggactacagtgccttt |
| | gactcttcacacggcactggctccttcgaggctctcatctctcactttttcaccgtggacaatggtttta |
| | gccctgcgctgggaccgtatctcagatccctggctgtctcggtgcacgcttacggcgagcgtcgcatcaa |
| | gattaccggtggcctcccctccggttgtgccgcgaccagcctgctgaacacagtgctcaacaatgtgatc |
| | atcaggactgctctggcattgacttacaaggaatttgaatatgacatggttgatatcatcgcctacggtg |
| | acgaccttctggttggcacggattacgatctggacttcaatgaggtggcacgacgcgctgccaagttggg |
| | gtataagatgactcctgccaacaagggttctgtcttccctccgacttcctctctttccgatgctgttttt |
| | ctaaagcgcaaattcgtccaaaacaacgacggcttatacaaaccagttatggatttaaagaatttggaag |
| | ccatgctctcctacttcaaaccaggaacactactcgagaagctgcaatctgtttctatgttggctcaaca |
| | ttctggaaaagaagaatatgatagattgatgcaccccttcgctgactacggtgccgtaccgagtcacgag |
| | tacctgcaggcaagatggagggccttgttcgactga |

TABLE 25
cDNA and amino acid sequences and corresponding SEQ ID NOs for the eight gene panel plus RPLPO.

| | Gene | | |
|---|---|---|---|
| | Acc- | | |
| Gene | ession | | |
| Name | number | cDNA sequence | Amino acid sequence |
| EFS | NM_ | ATGGCCATTGCCACGTCGACCCAGCTGGCCCGGGCA | MAIATSTQLARALYDNTAESPQELSFRRGDVLRVLQR |
| | 005864.4 | CTGTATGACAACACCGCTGAGTCCCCCCAGGAGC | EGAGGLDGWCLCSLHGQQGIVPANRVKLLPAGP |
| | | TGTCCTTCCGCCGAGGGGATGTCCTACGGGTCCTGC | APKPSLSPASPAQPGSPYPAPDHSNEDQEVYVVPPPAR |
| | | AGAGAGAGGGCGCTGGTGGACTGGACGGCTGGTG | PCPTSGPPAGPCPPSPDLIYKIPRASGTQLAA |
| | | CCTCTGCTCCCTACACGGCCAGCAGGGCATTGTGCC | PRDALEVYDVPPTALRVPSSGPYDCPASFSHPLTRVAP |
| | | CGCCAACAGGGTGAAGCTCTTGCCTGCTGGCCCA | QPPGEDDAPYDVPLTPKPPAELEPDLEWEGGR |
| | | GCACCCAAGCCCAGCCTCTCTCCTGCGTCCCCAGCC | EPGPPIYAAPSNLKRASALLNLYEAPEELLADGEGGGT |
| | | CAGCCTGGCTCACCATATCCAGCCCCAGATCACA | DEGIYDVPLLGPEAPPSPEPPGALASHDQDTL |
| | | GCAATGAGGACCAGGAGGTGTATGTGGTGCCGCCC | AQLLARSPPPPHRPRLPSAESLSRRPLPALPVPEAPSPSP |
| | | CCAGCTCGGCCCTGTCCAACCTCAGGACCTCCAGC | VPSPAPGRKGSIQDRPLPPPPPRLPGYGGP |
| | | TGGACCTTGCCCACCCTCTCCTGACCTCATCTACAA | KVEGDPEGREMEDDPAGHHNEYEGIPMAEEYDYVHL |
| | | AATCCCCAGAGCTAGTGGGACCCAGCTGGCTGCT | KGMDKAQGSRPPDQACTGDPELPERGMPAPQEAL |
| | | CCCAGAGATGCCTTGGAGGTCTACGATGTGCCCCCC | SPGEPLVVSTGDLQLLYFYAGQCQSHYSALQAAVAAL |
| | | ACCGCCCTCCGGGTGCCCTCCAGTGGCCCCTATG | MSSTQANQPPRLFVPHSKRVVVAAHRLVFVGDT |
| | | ACTGCCCTGCCTCCTTTTCCCACCCTCTGACCCGGGT | LGRLAASAPLRAQVRAAGTALGQALRATVLAVKGAA |
| | | TGCCCCGCAGCCCCCTGGAGAGGATGATGCTCC | LGYPSSPAIQEMVQCVTELAGQALQFTTLLTSLA |
| | | CTATGATGTGCCTCTGACCCCAAAGCCACCTGCAGA | P |
| | | GCTGGAACCAGATCTGGAGTGGGAAGGAGGCCGG | SEQ ID NO: 4 |
| | | GAGCCGGGGCCCCCCATCTATGCTGCCCCCTCCAAC | |
| | | CTGAAACGAGCGTCAGCCTTACTCAATTTGTATG | |
| | | AAGCACCCGAGGAACTGCTGGCAGACGGGGAGGGC | |
| | | GGGGGCACTGATGAGGGGATCTACGATGTGCCTCT | |
| | | GCTGGGGCCAGAGGCTCCCCCTTCTCCAGAGCCCCC | |
| | | TGGAGCCTTGGCCTCCCATGACCAGGACACCCTG | |
| | | GCCCAGCTTCTGGCCAGAAGCCCCCCACCCCCACAC | |
| | | AGGCCCCGGCTCCCCTCAGCTGAGAGCCTGTCCC | |
| | | GCCGCCCTCTGCCTGCCCTGCCTGTCCCTGAGGCCC | |
| | | CCAGCCCCTCCCCAGTGCCCTCTCCTGCCCCAGG | |
| | | CCGGAAGGGCAGCATCCAGGACCGGCCTCTGCCCCC | |
| | | ACCCCCACCCCGCCTGCCTGGTTATGGAGGCCCC | |
| | | AAGGTCGAGGGGGATCCAGAGGGCAGGGAGATGGA | |
| | | GGATGACCCAGCAGGACACCACAATGAGTACGAGG | |
| | | GCATTCCGATGGCCGAGGAGTATGACTATGTCCACC | |
| | | TGAAGGGCATGGACAAAGCTCAGGGATCTAGGCC | |
| | | CCCGGATCAGGCCTGCACAGGGGATCCTGAACTGCC | |
| | | CGAGAGGGGGATGCCGGCGCCGCAGGAGGCCCTG | |
| | | TCCCCAGGGGAGCCACTGGTTGTGTCCACCGGAGAT | |
| | | CTGCAGCTCCTGTACTTCTATGCTGGGCAATGCC | |
| | | AGAGCCACTACTCAGCCCTGCAGGCAGCCGTGGCA | |
| | | GCCCTGATGTCCAGTACCCAGGCTAATCAGCCCCC | |
| | | GCGCCTTTTCGTGCCCCACAGCAAGAGGGTGGTGGT | |
| | | GGCTGCTCATCGCCTGGTGTTTGTTGGGGACACC | |
| | | CTGGGCCGGCTGGCAGCCTCTGCCCCTCTGAGAGCA | |
| | | CAGGTCAGGGCTGCAGGTACAGCACTGGGCCAGG | |
| | | CATTGCGGGCCACTGTGCTGGCTGTCAAGGGAGCTG | |
| | | CCCTGGGCTACCCATCCAGCCCTGCCATCCAAGA | |
| | | GATGGTGCAGTGTGTAACAGAACTGGCAGGGCAGG | |
| | | CCCTGCAATTCACTACCCTGCTCACTAGCCTGGCT | |
| | | CCATGA | |
| | | SEQ ID NO: 3 | |
| MAFP4 | NM_ | ATGAAGGCACTCCTGGCCCTGCCGCTGCTGCTGCTT | MKALLALPLLLLLSTPPCAPQVSGIRGDALERFCLQQP |
| | 002404.3 | CTCTCCACGCCCCCGTGTGCCCCCCAGGTCTCCG | LDCDDIYAQGYQSDGVYLIYPSGPSVPVPVFC |
| | | GGATCCGAGGAGATGCTCTGGAGAGGTTTTGCCTTC | DMTTEGGKWTVFQKRFNGSVSFFRGWNDYKLGFGRA |
| | | AGCAACCCCTGGACTGTGACGACATCTATGCCCA | DGEYWLGLQNMHLLTLKQKYELRVDLEDFENNTA |
| | | GGGCTACCAGTCAGACGGCGTGTACCTCATCTACCC | YAKYADFSISPNAVSAEEDGYTLFVAGFEDGGAGDSL |
| | | CTCGGGCCCCAGTGTGCCTGTGCCCGTCTTCTGT | SYHSGQKFSTFDRDQDLFVQNCAALSSGAFWFR |
| | | GACATGACCACCGAGGGCGGGAAGTGGACGGTTTT | SCHFANLNGFYLGGSHLSYANGINWAQWKGFYYSLK |
| | | CCAGAAGAGATTCAATGGCTCAGTAAGTTTCTTCC | RTEMKIRRA |
| | | GCGGCTGGAATGACTACAAGCTGGGCTTCGGCCGTG | SEQ ID NO: 6 |
| | | CTGATGGAGAGTACTGGCTGGGGCTGCAGAACAT | |
| | | GCACCTCCTGACACTGAAGCAGAAGTATGAGCTGCG | |
| | | AGTGGACTTGGAGGACTTTGAGAACAACACGGCC | |
| | | TATGCCAAGTACGCTGACTTCTCCATCTCCCCGAAC | |
| | | GCGGTCAGCGCAGAGGAGGATGGCTACACCCTCT | |
| | | TTGTGGCAGGCTTTGAGGATGGCGGGGCAGGTGACT | |
| | | CCCTGTCCTACCACAGTGGCCAGAAGTTCTCTAC | |
| | | CTTCGACCGGGACCAGGACCTCTTTGTGCAGAACTG | |
| | | CGCAGCTCTCTCCTCAGGAGCCTTCTGGTTCCGC | |
| | | AGCTGCCACTTTGCCAACCTCAATGGCTTCTACCTA | |
| | | GGTGGCTCCCACCTCTCTTATGCCAATGGCATCA | |
| | | ACTGGGCCCAGTGGAAGGGCTTCTACTACTCCCTCA | |
| | | AACGCACTGAGATGAAAATCCGCCGGGCCTGA | |
| | | SEQ ID NO: 5 | |
| NTN3 | NM_ | ATGCCTGGCTGGCCCTGGGGGCTGCTGCTGACGGCA | MPGWPWGLLLTAGTLFAALSPGPPAPADPCHDEGGAP |
| | 006181.3 | GGCACGCTCTTCGCCGCCCTGAGTCCTGGGCCGC | RGCVPGLVNAALGREVLASSTCGRPATRACDAS |
| | | CGGCGCCCGCCGACCCCTGCCACGATGAGGGGGGT | DPRRAHSPALLTSPGGTASPLCWRSESLPRAPLNVTLT |
| | | GCGCCCCGCGGCTGCGTGCCAGGACTGGTGAACGC | VPLGKAFELVFVSLRFCSAPPASVALLKSQDH |
| | | CGCCCTGGGCCGCGAGGTGCTGGCTTCCAGCACGTG | GRSWAPLGFFSSHCDLDYGRLPAPANGPAGPGPEALC |
| | | CGGGCGGCCGGCCACTCGGGCCTGCGACGCCTCC | FPAPLAQPDGSGLLAFSMQDSSPPGLDLDSSPV |
| | | GACCCGCGACGGGCACACTCCCCCGCCCTCCTTACT | LQDWVTATDVRVVLTRPSTAGDPRDMEAVVPYSYAA |
| | | TCCCCAGGGGGCACGGCCAGCCCTCTGTGCTGGC | TDLQVGGRCKCNGHASRCLLDTQGHLICDCRHGT |
| | | GCTCGGAGTCCCTGCCTCGGGCGCCCCTCAACGTGA | EGPDCGRCKPFYCDRPWQRATARESHACLACSCNGH |
| | | CTCTCACGGTGCCCCTGGGCAAGGCTTTTGAGCT | ARRCRFNMELYRLSGRRSGGVCLNCRHNTAGRHC |
| | | GGTCTTCGTGAGCCTGCGCTTCTGCTCAGCTCCCCCA | HYCREGFYRDPGRALSDRRACRACDCHPVGAAGKTC |
| | | GCCTCCGTGGCCCTGCTCAAGTCTCAGGACCAT | NQTTGQCPCKDGVTGLTCNRCAPGFQQSRSPVAP |
| | | GGCCGCAGCTGGGCCCCGCTGGGCTTCTTCTCCTCC | CVKTPIPGPTEDSSPVQPQDCDSHCKPARGSYRISLKKF |
| | | CACTGTGACCTGGACTATGGCCGTCTGCCTGCCC | CKKDYAVQVAVGARGEARGAWTRFPVAVLAV |
| | | CTGCCAATGGCCCAGCTGGCCCAGGGCCTGAGGCCC | FRSGEERARRGSSALWVPAGDAACGCPRLLPGRRYLL |
| | | TGTGCTTCCCCGCACCCCTGGCCCAGCCTGATGG | LGGGPGAAAGGAGGRGPGLIAARGSLVLPWRDA |
| | | CAGCGGCCTTCTGGCCTTCAGCATGCAGGACAGCAG | WTRRLRRLQRRERRGRCSAA |
| | | CCCCCCAGGCCTGGACCTGGACAGCAGCCCAGTG | SEQ ID NO: 8 |
| | | CTCCAAGACTGGGTGACCGCCACCGACGTCCGTGTA | |
| | | GTGCTCACAAGGCCTAGCACGGCAGGTGACCCCA | |
| | | GGGACATGGAGGCCGTCGTCCCTTACTCCTACGCAG | |
| | | CCACCGACCTCCAGGTGGGCGGGCGCTGCAAGTG | |
| | | CAATGGACATGCCTCACGGTGCCTGCTGGACACACA | |
| | | GGGCCACCTGATCTGCGACTGTCGGCATGGCACC | |
| | | GAGGGCCCTGACTGCGGCCGCTGCAAGCCCTTCTAC | |
| | | TGCGACAGGCCATGGCAGCGGGCCACTGCCCGGG | |
| | | AATCCCACGCCTGCCTCGCTTGCTCCTGCAACGGCC | |
| | | ATGCCCGCCGCTGCCGCTTCAACATGGAGCTGTA | |
| | | CCGACTGTCCGGCCGCCGCAGCGGGGGTGTCTGTCT | |
| | | CAACTGCCGGCACAACACCGCCGGCCGCCACTGC | |
| | | CACTACTGCCGGGAGGGCTTCTATCGAGACCCTGGC | |
| | | CGTGCCCTGAGTGACCGTCGGGCTTGCAGGGCCT | |
| | | GCGACTGTCACCCGGTTGGTGCTGCTGGCAAGACCT | |
| | | GCAACCAGACCACAGGCCAGTGTCCCTGCAAGGA | |
| | | TGGCGTCACTGGCCTCACCTGCAACCGCTGCGCGCC | |
| | | TGGCTTCCAGCAAAGCCGCTCCCCAGTGGCGCCC | |
| | | TGTGTTAAGACCCCTATCCCTGGACCCACTGAGGAC | |
| | | AGCAGCCCTGTGCAGCCCCAGGACTGTGACTCGC | |
| | | ACTGCAAACCTGCCCGTGGCAGCTACCGCATCAGCC | |
| | | TAAAGAAGTTCTGCAAGAAGGACTATGCGGTGCA | |
| | | GGTGGCGGTGGGTGCGCGCGGCGAGGCGCGCGGCG | |
| | | CGTGGACACGCTTCCCGGTGGCGGTGCTCGCCGTG | |
| | | TTCCGGAGCGGAGAGGAGCGCGCGCGGCGCGGGAG | |
| | | TAGCGCGCTGTGGGTGCCCGCCGGGGATGCGGCCT | |
| | | GCGGCTGCCCGCGCCTGCTCCCCGGCCGCCGCTACC | |
| | | TCCTGCTGGGGGGCGGGCCTGGAGCCGCGGCTGG | |
| | | GGGCGCGGGGGGCCGGGGGCCCGGGCTCATCGCCG | |
| | | CCCGCGGAAGCCTCGTGCTACCCTGGAGGGACGCG | |
| | | TGGACGCGGCGCCTGCGGAGGCTGCAGCGACGCGA | |
| | | ACGGCGGGGGCGCTGCAGCGCCGCCTGA | |
| | | SEQ ID NO: 7 | |
| SOX11 | NM_ | ATGGTGCAGCAGGCGGAGAGCTTGGAAGCGGAGAG | MVQQAESLEAESNLPREALDTEEGEFMACSPVALDES |
| | 003108.4 | CAACCTGCCCCGGGAGGCGCTGGACACGGAGGAGG | DPDWCKTASGHIKRPMNAFMVWSKIERRKIMEQ |
| | | GCGAATTCATGGCTTGCAGCCCGGTGGCCCTGGACG | SPDMHNAEISKRLGKRWKMLKDSEKIPFIREAERLRLK |
| | | AGAGCGACCCAGACTGGTGCAAGACGGCGTCGGG | HMADYPDYKYRPRKKPKMDPSAKPSASQSPEK |
| | | CCACATCAAGCGGCCGATGAACGCGTTCATGGTATG | SAAGGGGGSAGGGAGGAKTSKGSSKKCGKLKAPAAA |
| | | GTCCAAGATCGAACGCAGGAAGATCATGGAGCAG | GAKAGAGKAAQSGDYGGAGDDYVLGSLRVSGSGG |
| | | TCTCCGGACATGCACAACGCCGAGATCTCCAAGAGG | GGAGKTVKCVFLDEDDDDDDDDDELQLQIKQEPDEE |
| | | CTGGGCAAGCGCTGGAAAATGCTGAAGGACAGCG | DEEPPHQQLLQPPGQQPSQLLRRYNVAKVPASPT |
| | | AGAAGATCCCGTTCATCCGGGAGGCGGAGCGGCTG | LSSSAESPEGASLYDEVRAGATSGAGGGSRLYYSFKNI |
| | | CGGCTCAAGCACATGGCCGACTACCCCGACTACAA | TKQHPPPLAQPALSPASSRSVSTSSSSSSGSS |
| | | GTACCGGCCCCGGAAAAAGCCCAAAATGGACCCCT | SGSSGEDADDLMFDLSLNFSQSAHSASEQQLGGGAAA |
| | | CGGCCAAGCCCAGCGCCAGCCAGAGCCCAGAGAAG | GNLSLSLVDKDLDSFSEGSLGSHFEFPDYCTPE |
| | | AGCGCGGCCGGCGGCGGCGGCGGGAGCGCGGGCGG | LSEMIAGDWLEANFSDLVFTY |
| | | AGGCGCGGGCGGTGCCAAGACCTCCAAGGGCTCCA | SEQ ID NO: 10 |
| | | GCAAGAAATGCGGCAAGCTCAAGGCCCCCGCGGCC | |
| | | GCGGGCGCCAAGGCGGGCGCGGGCAAGGCGGCCCA | |
| | | GTCCGGGGACTACGGGGGCGCGGGCGACGACTACG | |
| | | TGCTGGGCAGCCTGCGCGTGAGCGGCTCGGGCGGC | |
| | | GGCGGCGCGGGCAAGACGGTCAAGTGCGTGTTTCTG | |
| | | GATGAGGACGACGACGACGACGACGACGACGACG | |
| | | AGCTGCAGCTGCAGATCAAACAGGAGCCGGACGAG | |
| | | GAGGACGAGGAACCACCGCACCAGCAGCTCCTGCA | |
| | | GCCGCCGGGGCAGCAGCCGTCGCAGCTGCTGAGAC | |
| | | GCTACAACGTCGCCAAAGTGCCCGCCAGCCCTACG | |
| | | CTGAGCAGCTCGGCGGAGTCCCCCGAGGGAGCGAG | |
| | | CCTCTACGACGAGGTGCGGGCCGGCGCGACCTCGG | |
| | | GCGCCGGGGGCGGCAGCCGCCTCTACTACAGCTTCA | |
| | | AGAACATCACCAAGCAGCACCCGCCGCCGCTCGC | |
| | | GCAGCCCGCGCTGTCGCCCGCGTCCTCGCGCTCGGT | |
| | | GTCCACCTCCTCGTCCAGCAGCAGCGGCAGCAGC | |
| | | AGCGGCAGCAGCGGCGAGGACGCCGACGACCTGAT | |
| | | GTTCGACCTGAGCTTGAATTTCTCTCAAAGCGCGC | |
| | | ACAGCGCCAGCGAGCAGCAGCTGGGGGGCGGCGCG | |
| | | GCGGCCGGGAACCTGTCCCTGTCGCTGGTGGATAA | |
| | | GGATTTGGATTCGTTCAGCGAGGGCAGCCTGGGCTC | |
| | | CCACTTCGAGTTCCCCGACTACTGCACGCCGGAG | |
| | | CTGAGCGAGATGATCGCGGGGGACTGGCTGGAGGC | |
| | | GAACTTCTCCGACCTGGTGTTCACATATTGA | |
| | | SEQ ID NO: 9 | |
| NEFH | NM_ | ATGATGAGCTTCGGCGGCGCGGACGCGCTGCTGGGC | MMSFGGADALLGAPFAPLHGGGSLHYALARKGGAGG |
| | 021076.4 | GCCCCGTTCGCGCCGCTGCATGGCGGCGGCAGCC | TRSAAGSSSGFHSWTRTSVSSVSASPSRFRGAGA |
| | | TCCACTACGCGCTAGCCCGAAAGGGTGGCGCAGGC | ASSTDSLDTLSNGPEGCMVAVATSRSEKEQLQALNDR |
| | | GGGACGCGCTCCGCCGCTGGCTCCTCCAGCGGCTT | FAGYIDKVRQLEAHNRSLEGEAAALRQQQAGRS |
| | | CCACTCGTGGACACGGACGTCCGTGAGCTCCGTGTC | AMGELYEREVREMRGAVLRLGAARGQLRLEQEHLLE |
| | | CGCCTCGCCCAGCCGCTTCCGTGGCGCAGGCGCC | DIAHVRQRLDDEARQREEAEAAARALARFAQEAE |
| | | GCCTCAAGCACCGACTCGCTGGACACGCTGAGCAAC | AARVDLQKKAQALQEECGYLRRHHQEEVGELLGQIQ |
| | | GGGCCGGAGGGCTGCATGGTGGCGGTGGCCACCT | GSGAAQAQMQAETRDALKCDVTSALREIRAQLEG |
| | | CACGCAGTGAGAAGGAGCAGCTGCAGGCGCTGAAC | HAVQSTLQSEEWFRVRLDRLSEAAKVNTDAMRSAQE |
| | | GACCGCTTCGCCGGGTACATCGACAAGGTGCGGCA | EITEYRRQLQARTTELEALKSTKDSLERQRSELE |
| | | GCTGGAGGCGCACAACCGCAGCCTGGAGGGCGAGG | DRHQADIASYQEAIQQLDAELRNTKWEMAAQLREYQ |
| | | CTGCGGCGCTGCGGCAGCAGCAGGCGGGCCGCTCC | DLLNVKMALDIEIAAYRKLLEGEECRIGFGPIPF |
| | | GCTATGGGCGAGCTGTACGAGCGCGAGGTCCGCGA | SLPEGLPKIPSVSTHIKVKSEEKIKVVEKSEKETVIVEEQ |
| | | GATGCGCGGCGCGGTGCTGCGCCTGGGCGCGGCGC | TEETQVTEEVTEEEEKEAKEEEGKEEEGGE |
| | | GCGGTCAGCTACGCCTGGAGCAGGAGCACCTGCTCG | EEEAEGGEEETKSPPAEEAASPEKEAKSPVKEEAKSPA |
| | | AGGACATCGCGCACGTGCGCCAGCGCCTAGACGA | EAKSPEKEEAKSPAEVKSPEKAKSPAKEEAKS |
| | | CGAGGCCCGGCAGCGAGAGGAGGCCGAGGCGGCGG | PPEAKSPEKEEAKSPAEVKSPEKAKSPAKEEAKSPAEA |
| | | CCCGCGCGCTGGCGCGCTTCGCGCAGGAGGCCGAG | KSPEKAKSPVKEEAKSPAEAKSPVKEEAKSPA |
| | | GCGGCGCGCGTGGACCTGCAGAAGAAGGCGCAGGC | EVKSPEKAKSPTKEEAKSPEKAKSPEKEEAKSPEKAKS |
| | | GCTGCAGGAGGAGTGCGGCTACCTGCGGCGCCACC | PVKAEAKSPEKAKSPVKAEAKSPEKAKSPVKE |
| | | ACCAGGAAGAGGTGGGCGAGCTGCTCGGCCAGATC | EAKSPEKAKSPVKEEAKSPEKAKSPVKEEAKTPEKAKS |
| | | CAGGGCTCCGGCGCCGCGCAGGCGCAGATGCAGGC | PVKEEAKSPEKAKSPEKAKTLDVKSPEAKTPA |
| | | CGAGACGCGCGACGCCCTGAAGTGCGACGTGACGT | KEEARSPADKFPEKAKSPVKEEVKSPEKAKSPLKEDA |
| | | CGGCGCTGCGCGAGATTCGCGCGCAGCTTGAAGGC | KAPEKEIPKKEEVKSPVKEEEKPQEVKVKEPPK |
| | | CACGCGGTGCAGAGCACGCTGCAGTCCGAGGAGTG | KAEEEKAPATPKTEEKKDSKKEEAPKKEAPKPKVEEK |
| | | GTTCCGAGTGAGGCTGGACCGACTGTCGGAGGCAG | KEPAVEKPKESKVEAKKEEAEDKKKVPTPEKEA |
| | | CCAAGGTGAACACAGACGCTATGCGCTCAGCGCAG | PAKVEVKEDAKPKEKTEVAKKEPDDAKAKEPSKPAE |
| | | GAGGAGATAACTGAGTACCGGCGTCAGCTGCAGGC | KKEAAPEKKDTKEEKAKKPEEKPKTEAKAKEDDK |
| | | CAGGACCACAGAGCTGGAGGCACTGAAAAGCACCA | TLSKEPSKPKAEKAEKSSSTDQKDSKPPEKATEDKAAK |
| | | AGGACTCACTGGAGAGGCAGCGCTCTGAGCTGGAG | GK |
| | | GACCGTCATCAGGCCGACATTGCCTCCTACCAGGAA | SEQ ID NO: 12 |
| | | GCCATTCAGCAGCTGGACGCTGAGCTGAGGAACA | |
| | | CCAAGTGGGAGATGGCCGCCCAGCTGCGAGAATAC | |
| | | CAGGACCTGCTCAATGTCAAGATGGCTCTGGATAT | |
| | | AGAGATAGCCGCTTACAGAAAACTCCTGGAAGGTG | |
| | | AAGAGTGTCGGATTGGCTTTGGCCCAATTCCTTTC | |
| | | TCGCTTCCAGAAGGACTCCCCAAAATTCCCTCTGTG | |
| | | TCCACTCACATAAAGGTGAAAAGCGAAGAGAAGA | |
| | | TCAAAGTGGTGGAGAAGTCTGAGAAAGAAACTGTG | |
| | | ATTGTGGAGGAACAGACAGAGGAGACCCAAGTGAC | |
| | | TGAAGAAGTGACTGAAGAAGAGGAGAAAGAGGCCA | |
| | | AAGAGGAGGAGGGCAAGGAGGAAGAAGGGGGTGA | |
| | | A | |
| | | GAAGAGGAGGCAGAAGGGGGAGAAGAAGAAACAA | |
| | | AGTCTCCCCCAGCAGAAGAGGCTGCATCCCCAGAG | |
| | | A | |
| | | AGGAAGCCAAGTCACCAGTAAAGGAAGAGGCAAAG | |
| | | TCACCGGCTGAGGCCAAGTCCCCAGAGAAGGAGGA | |
| | | AGCAAAATCCCCAGCCGAAGTCAAGTCCCCTGAGA | |
| | | AGGCCAAGTCTCCAGCAAAGGAAGAGGCAAAGTCA | |
| | | CCGCCTGAGGCCAAGTCCCCAGAGAAGGAGGAAGC | |
| | | AAAATCTCCAGCTGAGGTCAAGTCCCCCGAGAAGG | |
| | | CCAAGTCCCCAGCAAAGGAAGAGGCAAAGTCACCG | |
| | | GCTGAGGCCAAGTCTCCAGAGAAGGCCAAGTCCCC | |
| | | AGTGAAGGAAGAAGCAAAGTCACCGGCTGAGGCCA | |
| | | AGTCCCCAGTGAAGGAAGAAGCAAAATCTCCAGCT | |
| | | GAGGTCAAGTCCCCGGAAAAGGCCAAGTCTCCAAC | |
| | | GAAGGAGGAAGCAAAGTCCCCTGAGAAGGCCAAGT | |
| | | CCCCAGAGAAGGAAGAGGCCAAGTCCCCTGAGAAG | |
| | | GCCAAGTCCCCAGTGAAGGCAGAAGCAAAGTCCCC | |
| | | TGAGAAGGCCAAGTCCCCAGTGAAGGCAGAAGCAA | |
| | | AGTCCCCTGAGAAGGCCAAGTCCCCAGTGAAGGAA | |
| | | GAAGCAAAGTCCCCTGAGAAGGCCAAGTCCCCAGT | |
| | | GAAGGAAGAAGCAAAGTCCCCTGAGAAGGCCAAGT | |
| | | CCCCAGTGAAGGAAGAAGCAAAGACCCCCGAGAAG | |
| | | GCCAAGTCCCCAGTGAAGGAAGAAGCTAAGTCCCC | |
| | | AGAGAAGGCCAAGTCCCCAGAGAAGGCCAAGACTC | |
| | | TTGATGTGAAGTCTCCAGAAGCCAAGACTCCAGCG | |
| | | AAGGAGGAAGCAAGGTCCCCTGCAGACAAATTCCC | |
| | | TGAAAAGGCCAAAAGCCCTGTCAAGGAGGAGGTCA | |
| | | AGTCCCCAGAGAAGGCGAAATCTCCCCTGAAGGAG | |
| | | GATGCCAAGGCCCCTGAGAAGGAGATCCCAAAAAA | |
| | | GGAAGAGGTGAAGTCCCCAGTGAAGGAGGAGGAGA | |
| | | AGCCCCAGGAGGTGAAAGTCAAAGAGCCCCCAAAG | |
| | | AAGGCAGAGGAAGAGAAAGCCCCTGCCACACCAAA | |
| | | AACAGAGGAGAAGAAGGACAGCAAGAAAGAGGAG | |
| | | G | |
| | | CACCCAAGAAGGAGGCTCCAAAGCCCAAGGTGGAG | |
| | | GAGAAGAAGGAACCTGCTGTCGAAAAGCCCAAAGA | |
| | | ATCCAAAGTTGAAGCCAAGAAGGAAGAGGCTGAAG | |
| | | ATAAGAAAAAAGTCCCCACCCCAGAGAAGGAGGCT | |
| | | CCTGCCAAGGTGGAGGTGAAGGAAGACGCTAAACC | |
| | | CAAAGAAAAGACAGAGGTAGCCAAGAAGGAACCA | |
| | | G | |
| | | ATGATGCCAAGGCCAAGGAACCCAGCAAACCAGCA | |
| | | GAGAAGAAGGAGGCAGCACCGGAGAAAAAAGACA | |
| | | C | |
| | | CAAGGAGGAGAAGGCCAAGAAGCCTGAGGAGAAA | |
| | | CCCAAGACAGAGGCCAAAGCCAAGGAAGATGACAA | |
| | | G | |
| | | ACCCTCTCAAAAGAGCCTAGCAAGCCTAAGGCAGA | |
| | | AAAGGCTGAAAAATCCTCCAGCACAGACCAAAAAG | |
| | | ACAGCAAGCCTCCAGAGAAGGCCACAGAAGACAAG | |
| | | GCCGCCAAGGGGAAGTAA SEQ ID NO: 11 | |
| CNTFR | NM_ | ATGGCTGCTCCTGTCCCGTGGGCCTGCTGTGCTGTG | MAAPVPWACCAVLAAAAAVVYAQRHSPQEAPHVQY |
| | 147164.3 | CTTGCCGCCGCCGCCGCAGTTGTCTACGCCCAGA | ERLGSDVTLPCGTANWDAAVTWRVNGTDLAPDLLN |
| | | GACACAGTCCACAGGAGGCACCCCATGTGCAGTAC | GSQLVLHGLELGHSGLYACFHRDSWHLRHQVLLHVG |
| | | GAGCGCCTGGGCTCTGACGTGACACTGCCATGTGG | LPPREPVLSCRSNTYPKGFYCSWHLPTPTYIPNT |
| | | GACAGCAAACTGGGATGCTGCGGTGACGTGGCGGG | FNVTVLHGSKIMVCEKDPALKNRCHIRYMHLFSTIKY |
| | | TAAATGGGACAGACCTGGCCCCTGACCTGCTCAAC | KVSISVSNALGHNATAITFDEFTIVKPDPPENV |
| | | GGCTCTCAGCTGGTGCTCCATGGCCTGGAACTGGGC | VARPVPSNPRRLEVTWQTPSTWPDPESFPLKFFLRYRP |
| | | CACAGTGGCCTCTACGCCTGCTTCCACCGTGACT | LILDQWQHVELSDGTAHTITDAYAGKEYIIQV |
| | | CCTGGCACCTGCGCCACCAAGTCCTGCTGCATGTGG | AAKDNEIGTWSDWSVAAHATPWTEEPRHLTTEAQAA |
| | | GCTTGCCGCCGCGGGAGCCTGTGCTCAGCTGCCG | ETTTSTTSSLAPPPTTKICDPGELGSGGGPSAPF |
| | | CTCCAACACTTACCCCAAGGGCTTCTACTGCAGCTG | LVSVPITLALAAAAATASSLLI |
| | | GCATCTGCCCACCCCCACCTACATTCCCAACACC | SEQ ID NO: 14 |
| | | TTCAATGTGACTGTGCTGCATGGCTCCAAAATTATG | |
| | | GTCTGTGAGAAGGACCCAGCCCTCAAGAACCGCT | |
| | | GCCACATTCGCTACATGCACCTGTTCTCCACCATCA | |
| | | AGTACAAGGTCTCCATAAGTGTCAGCAATGCCCT | |
| | | GGGCCACAATGCCACAGCTATCACCTTTGACGAGTT | |
| | | CACCATTGTGAAGCCTGATCCTCCAGAAAATGTG | |
| | | GTAGCCCGGCCAGTGCCCAGCAACCCTCGCCGGCTG | |
| | | GAGGTGACGTGGCAGACCCCCTCGACCTGGCCTG | |
| | | ACCCTGAGTCTTTTCCTCTCAAGTTCTTTCTGCGCTA | |
| | | CCGACCCCTCATCCTGGACCAGTGGCAGCATGT | |
| | | GGAGCTGTCCGACGGCACAGCACACACCATCACAG | |
| | | ATGCCTACGCCGGGAAGGAGTACATTATCCAGGTG | |
| | | GCAGCCAAGGACAATGAGATTGGGACATGGAGTGA | |
| | | CTGGAGCGTAGCCGCCCACGCTACGCCCTGGACTG | |
| | | AGGAACCGCGACACCTCACCACGGAGGCCCAGGCT | |
| | | GCGGAGACCACGACCAGCACCACCAGCTCCCTGGC | |
| | | ACCCCCACCTACCACGAAGATCTGTGACCCTGGGGA | |
| | | GCTGGGCAGCGGCGGGGGACCCTCGGCACCCTTC | |
| | | TTGGTCAGCGTCCCCATCACTCTGGCCCTGGCTGCC | |
| | | GCTGCCGCCACTGCCAGCAGTCTCTTGATCTGA | |
| | | SEQ ID NO: 13 | |
| JPH4 | NM_ | ATGTCCCCCGGGGGCAAGTTCGACTTTGACGACGGG | MSPGGKFDFDDGGCYVGGWEAGRAHGYGVCTGPGA |
| | 0011460 | GGCTGCTACGTGGGGGGCTGGGAGGCGGGGGGGG | QGEYSGCWAHGFESLGVFTGPGGHSYQGHWQQGKR |
| | 28.2 | CACATGGCTACGGCGTGTGCACGGGCCCCGGCGCCC | EGLGVERKSRWTYRGEWLGGLKGRSGVWESVSGLRY |
| | | AGGGCGAGTACAGCGGCTGCTGGGCACACGGCTT | AGLWKDGFQDGYGTETYSDGGTYQGQWQAGKRHG |
| | | CGAGTCACTGGGCGTCTTCACGGGGCCCGGCGGACA | YGVRQSVPYHQAALLRSPRRTSLDSGHSDPPTPPPPLP |
| | | CAGCTACCAGGGCCACTGGCAGCAGGGCAAGCGC | LPGDEGGSPASGSRGGFVLAGPGDADGASSRK |
| | | GAAGGGCTGGGCGTGGAGCGCAAGAGCCGCTGGAC | RTPAAGGFFRRSLLLSGLRAGGRRSSLGSKRGSLRSEV |
| | | GTACCGCGGCGAGTGGCTGGGCGGGCTGAAGGGGC | SSEVGSTGPPGSEASGPPAAAPPALIEGSATE |
| | | GCAGCGGCGTGTGGGAAAGCGTGTCCGGCCTGCGCT | VYAGEWRADRRSGFGVSQRSNGLRYEGEWLGNRRH |
| | | ACGCCGGGCTCTGGAAGGACGGTTTCCAGGACGG | GYGRTTRPDGSREEGKYKRNRLVHGGRVRSLLPLA |
| | | CTACGGCACTGAGACCTACTCCGACGGAGGCACCTA | LRRGKVKEKVDRAVEGARRAVSAARQRQEIAAARAA |
| | | CCAGGGCCAGTGGCAGGCCGGGAAGCGCCACGGC | DALLKAVAASSVAEKAVEAARMAKLIAQDLQPML |
| | | TACGGGGTACGCCAGAGTGTGCCCTACCATCAGGCG | EAPGRRPRQDSEGSDTEPLDEDSPGVYENGLTPSEGSP |
| | | GCGCTGCTGCGCTCGCCCCGCCGCACCTCCCTGG | ELPSSPASSRQPWRPPACRSPLPPGGDQGPFS |
| | | ATTCCGGCCACAGCGACCCCCCGACGCCACCCCCGC | SPKAWPEEWGGAGAQAEELAGYEAEDEAGMQGPGP |
| | | CCCTGCCCTTGCCGGGCGACGAGGGAGGCAGCCC | RDGSPLLGGCSDSSGSLREEEGEDEEPLPPLRAPA |
| | | CGCCTCGGGCTCCCGGGGCGGCTTCGTGCTGGCCGG | GTEPEPIAMLVLRGSSSRGPDAGCLTEELGEPAATERP |
| | | GCCCGGGGACGCCGACGGCGCGTCGTCCCGAAAG | AQPGAANPLVVGAVALLDLSLAFLFSQLLT |
| | | CGCACTCCGGCGGCCGGCGGATTCTTTCGCCGTTCG | SEQ ID NO: 16 |
| | | CTGCTGCTCAGCGGGCTCCGAGCGGGCGGACGTC | |
| | | GCAGCTCCCTGGGCAGCAAGCGAGGCTCCCTGCGCA | |
| | | GCGAGGTGAGCAGCGAGGTGGGCAGCACCGGACC | |
| | | GCCCGGCTCGGAGGCCAGCGGGCCCCCGGCCGCAG | |
| | | CGCCGCCCGCCCTCATCGAGGGCTCGGCCACAGAG | |
| | | GTGTACGCGGGCGAGTGGCGCGCAGATCGGCGCAG | |
| | | CGGCTTCGGCGTCAGCCAGCGCTCCAACGGGCTGC | |
| | | GCTACGAGGGCGAGTGGCTGGGCAACCGGCGGCAC | |
| | | GGCTACGGGCGCACCACCCGCCCCGACGGCTCCCG | |
| | | CGAGGAGGGCAAGTACAAGCGCAACCGGCTGGTGC | |
| | | ACGGCGGGCGCGTCCGCAGTCTCCTGCCTCTGGCC | |
| | | CTTCGGCGGGGCAAGGTTAAGGAGAAGGTGGACAG | |
| | | GGCTGTCGAGGGCGCCCGTCGAGCCGTGAGTGCTG | |
| | | CCCGTCAGCGCCAGGAGATCGCCGCTGCCAGGGCA | |
| | | GCAGACGCCCTCCTAAAGGCAGTGGCAGCCAGCAG | |
| | | TGTCGCTGAGAAGGCCGTGGAGGCAGCTCGAATGG | |
| | | CCAAACTGATAGCCCAGGACCTGCAGCCCATGCTA | |
| | | GAGGCCCCAGGCCGCAGACCCAGGCAGGACTCAGA | |
| | | AGGTTCCGACACGGAGCCCCTGGATGAGGACAGCC | |
| | | CTGGGGTATATGAGAACGGACTGACCCCCTCAGAG | |
| | | GGATCCCCTGAACTGCCCAGCAGTCCTGCCTCCTC | |
| | | CCGCCAACCCTGGCGACCCCCTGCCTGCCGGAGCCC | |
| | | ACTGCCTCCTGGAGGGGACCAGGGTCCCTTCTCC | |
| | | AGCCCCAAAGCTTGGCCTGAGGAGTGGGGGGGGGC | |
| | | AGGCGCACAGGCAGAGGAACTAGCTGGCTATGAGG | |
| | | CTGAGGATGAGGCTGGGATGCAAGGGCCAGGGCCC | |
| | | AGAGACGGTTCCCCACTCCTCGGAGGCTGCAGCGA | |
| | | CAGTTCAGGAAGTCTTCGAGAGGAGGAGGGGGAGG | |
| | | ATGAAGAGCCCCTGCCCCCGCTGAGGGCCCCAGCA | |
| | | GGCACGGAGCCTGAGCCCATCGCCATGCTGGTCCTG | |
| | | AGGGGCTCGTCCTCGAGGGGTCCTGATGCTGGGT | |
| | | GCCTGACAGAAGAGCTCGGGGAGCCCGCTGCAACC | |
| | | GAGAGGCCTGCCCAGCCGGGAGCTGCCAACCCCCT | |
| | | GGTGGTGGGAGCCGTGGCCCTCCTGGACCTCAGCCT | |
| | | GGCATTCCTGTTCTCCCAGCTCCTCACCTGA | |
| | | SEQ ID NO: 15 | |
| ANT | NM_ | ATGGCCACGGCGGAGCGGAGAGCCCTCGGCATCGG | MATAERRALGIGFQWLSLATLVLICAGQGGRREDGGP |
| XR1 | 032208.3 | CTTCCAGTGGCTCTCTTTGGCCACTCTGGTGCTCA | ACYGGFDLYFILDKSGSVLHHWNEIYYFVEQLA |
| | | TCTGCGCCGGGCAAGGGGGACGCAGGGAGGATGGG | HKFISPQLRMSFIVFSTRGTTLMKLTEDREQIRQGLEEL |
| | | GGTCCAGCCTGCTACGGCGGATTTGACCTGTACTT | QKVLPGGDTYMHEGFERASEQIYYENRQGYR |
| | | CATTTTGGACAAATCAGGAAGTGTGCTGCACCACTG | TASVIIALTDGELHEDLFFYSEREANRSRDLGAIVYCV |
| | | GAATGAAATCTATTACTTTGTGGAACAGTTGGCT | GVKDFNETQLARIADSKDHVFPVNDGFQALQG |
| | | CACAAATTCATCAGCCCACAGTTGAGAATGTCCTTT | IIHSILKKSCIEILAAEPSTICAGESFQVVVRGNGFRHAR |
| | | ATTGTTTTCTCCACCCGAGGAACAACCTTAATGA | NVDRVLCSFKINDSVTLNEKPFSVEDTYLL |
| | | AACTGACAGAAGACAGAGAACAAATCCGTCAAGGC | CPAPILKEVGMKAALQVSMNDGLSFISSSVIITTTHCSD |
| | | CTAGAAGAACTCCAGAAAGTTCTGCCAGGAGGAGA | GSILAIALLILFLLLALALLWWFWPLCCTVI |
| | | CACTTACATGCATGAAGGATTTGAAAGGGCCAGTGA | IKEVPPPPAEESEEEDDDGLPKKKWPTVDASYYGGRG |
| | | GCAGATTTATTATGAAAACAGACAAGGGTACAGG | VGGIKRMEVRWGEKGSTEEGAKLEKAKNARVKM |
| | | ACAGCCAGCGTCATCATTGCTTTGACTGATGGAGAA | PEQEYEFPEPRNLNNNMRRPSSPRKWYSPIKGKLDAL |
| | | CTCCATGAAGATCTCTTTTTCTATTCAGAGAGGG | WVLLRKGYDRVSVMRPQPGDTGRCINFTRVKNN |
| | | AGGCTAATAGGTCTCGAGATCTTGGTGCAATTGTTT | QPAKYPLNNAYHTSSPPPAPIYTPPPPAPHCPPPPPSAPT |
| | | ACTGTGTTGGTGTGAAAGATTTCAATGAGACACA | PPIPSPPSTLPPPPQAPPPNRAPPPSRPPP |
| | | GCTGGCCCGGATTGCGGACAGTAAGGATCATGTGTT | RPSV |
| | | TCCCGTGAATGACGGCTTTCAGGCTCTGCAAGGC | SEQ ID NO: 18 |
| | | ATCATCCACTCAATTTTGAAGAAGTCCTGCATCGAA | |
| | | ATTCTAGCAGCTGAACCATCCACCATATGTGCAG | |
| | | GAGAGTCATTTCAAGTTGTCGTGAGAGGAAACGGCT | |
| | | TCCGACATGCCCGCAACGTGGACAGGGTCCTCTG | |
| | | CAGCTTCAAGATCAATGACTCGGTCACACTCAATGA | |
| | | GAAGCCCTTTTCTGTGGAAGATACTTATTTACTG | |
| | | TGTCCAGCGCCTATCTTAAAAGAAGTTGGCATGAAA | |
| | | GCTGCACTCCAGGTCAGCATGAACGATGGCCTCT | |
| | | CTTTTATCTCCAGTTCTGTCATCATCACCACCACACA | |
| | | CTGTTCTGACGGTTCCATCCTGGCCATCGCCCT | |
| | | GCTGATCCTGTTCCTGCTCCTAGCCCTGGCTCTCCTC | |
| | | TGGTGGTTCTGGCCCCTCTGCTGCACTGTGATT | |
| | | ATCAAGGAGGTCCCTCCACCCCCTGCCGAGGAGAGT | |
| | | GAGGAAGAAGATGATGATGGTCTGCCTAAGAAAA | |
| | | AGTGGCCAACGGTAGACGCCTCTTATTATGGTGGGA | |
| | | GAGGCGTTGGAGGCATTAAAAGAATGGAGGTTCG | |
| | | TTGGGGAGAAAAGGGCTCCACAGAAGAAGGTGCTA | |
| | | AGTTGGAAAAGGCAAAGAATGCAAGAGTCAAGATG | |
| | | CCGGAGCAGGAATATGAATTCCCTGAGCCGCGAAA | |
| | | TCTCAACAACAATATGCGTCGGCCTTCTTCCCCCC | |
| | | GGAAGTGGTACTCTCCAATCAAGGGAAAACTCGAT | |
| | | GCCTTGTGGGTCCTACTGAGGAAAGGATATGATCG | |
| | | TGTGTCTGTGATGCGTCCACAGCCAGGAGACACGGG | |
| | | GCGCTGCATCAACTTCACCAGGGTCAAGAACAAC | |
| | | CAGCCAGCCAAGTACCCACTCAACAACGCCTACCAC | |
| | | ACCTCCTCGCCGCCTCCTGCCCCCATCTACACTC | |
| | | CCCCACCTCCTGCGCCCCACTGCCCTCCCCCGCCCCC | |
| | | CAGCGCCCCTACCCCTCCCATCCCGTCCCCACC | |
| | | TTCCACCCTTCCCCCTCCTCCCCAGGCTCCACCTCCC | |
| | | AACAGGGCACCTCCTCCCTCCCGCCCTCCTCCA | |
| | | AGGCCTTCTGTCTAG | |
| | | SEQ ID NO: 17 | |
| RPLP0 | NM_ | ATGCCCAGGGAAGACAGGGCGACCTGGAAGTCCAA | MPREDRATWKSNYFLKIIQLLDDYPKCFIVGADNVGS |
| | 001002.4 | CTACTTCCTTAAGATCATCCAACTATTGGATGATT | KQMQQIRMSLRGKAVVLMGKNTMMRKAIRGHLE |
| | | ATCCGAAATGTTTCATTGTGGGAGCAGACAATGTGG | NNPALEKLLPHIRGNVGFVFTKEDLTEIRDMLLANKVP |
| | | GCTCCAAGCAGATGCAGCAGATCCGCATGTCCCT | AAARAGAIAPCEVTVPAQNTGLGPEKTSFFQA |
| | | TCGCGGGAAGGCTGTGGTGCTGATGGGCAAGAACA | LGITTKISRGTIEILSDVQLIKTGDKVGASEATLLNMLNI |
| | | CCATGATGCGCAAGGCCATCCGAGGGCACCTGGAA | SPFSFGLVIQQVFDNGSIYNPEVLDITEET |
| | | AACAACCCAGCTCTGGAGAAACTGCTGCCTCATATC | LHSRFLEGVRNVASVCLQIGYPTVASVPHSIINGYKRV |
| | | CGGGGGAATGTGGGCTTTGTGTTCACCAAGGAGG | LALSVETDYTFPLAEKVKAFLADPSAFVAAAP |
| | | ACCTCACTGAGATCAGGGACATGTTGCTGGCCAATA | VAAATTAAPAAAAAPAKVEAKEESEESDEDMGFGLF |
| | | AGGTGCCAGCTGCTGCCCGTGCTGGTGCCATTGC | D |
| | | CCCATGTGAAGTCACTGTGCCAGCCCAGAACACTGG | SEQ ID NO: 20 |
| | | TCTCGGGCCCGAGAAGACCTCCTTTTTCCAGGCT | |
| | | TTAGGTATCACCACTAAAATCTCCAGGGGCACCATT | |
| | | GAAATCCTGAGTGATGTGCAGCTGATCAAGACTG | |
| | | GAGACAAAGTGGGAGCCAGCGAAGCCACGCTGCTG | |
| | | AACATGCTCAACATCTCCCCCTTCTCCTTTGGGCT | |
| | | GGTCATCCAGCAGGTGTTCGACAATGGCAGCATCTA | |
| | | CAACCCTGAAGTGCTTGATATCACAGAGGAAACT | |
| | | CTGCATTCTCGCTTCCTGGAGGGTGTCCGCAATGTT | |
| | | GCCAGTGTCTGTCTGCAGATTGGCTACCCAACTG | |
| | | TTGCATCAGTACCCCATTCTATCATCAACGGGTACA | |
| | | AACGAGTCCTGGCCTTGTCTGTGGAGACGGATTA | |
| | | CACCTTCCCACTTGCTGAAAAGGTCAAGGCCTTCTT | |
| | | GGCTGATCCATCTGCCTTTGTGGCTGCTGCCCCT | |
| | | GTGGCTGCTGCCACCACAGCTGCTCCTGCTGCTGCT | |
| | | GCAGCCCCAGCTAAGGTTGAAGCCAAGGAAGAGT | |
| | | CGGAGGAGTCGGACGAGGATATGGGATTTGGTCTCT | |
| | | TTGACTAA | |

### Example 6. Cancer Therapies with Oncolytic Virus SVV-001 and Nivolumab for Screened Patients

The aim of this study is to determine the maximum tolerated dose (MTD) and recommended phase 2 dose (RP2D) of intratumorally injected SVV-001 in combination with the programmed death receptor 1 (PD-1) immune checkpoint blocker (ICB) nivolumab (Part I) in treatment of poorly differentiated neuroendocrine carcinomas (NECs) and well-differentiated neuroendocrine tumors (NETs). Throughout this disclosure, the terms immune checkpoint inhibitor and immune checkpoint blocker are used interchangeably.

This is a dose-escalation, and cohort-expansion study of SVV-001, administered intratumorally in combination with the ICB nivolumab, in patients with poorly differentiated NECs and well-differentiated NETs.

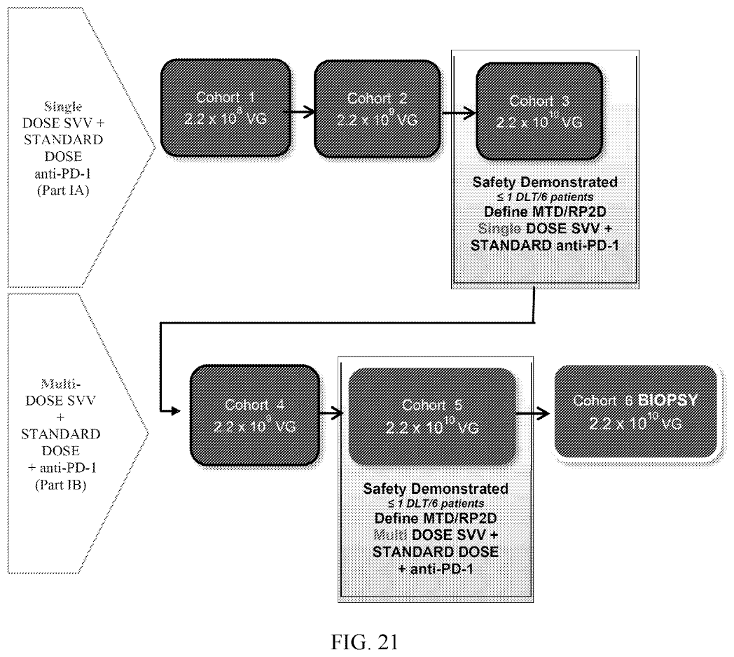

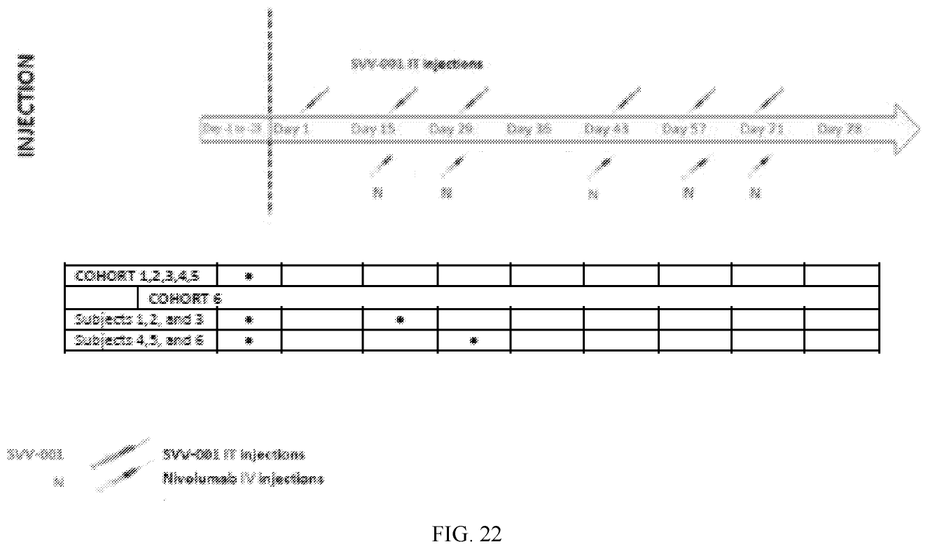

This trial is designed to establish the MTD and recommended RP2D of single and multiple doses of SVV-001 in combination with nivolumab. See FIG. 21 for a schematic of the trial and FIG. 22 for the injection and biopsy schedule.

The study is described in the following synopsis and protocol.

SYNOPSIS of a Phase 1 Trial of the Oncolytic Virus SVV-001 in Combination with Nivolumab in Patients with Poorly Differentiated Neuroendocrine Carcinomas and Well-Differentiated Neuroendocrine Tumors

### PROTOCOL NUMBER: SVV-001-003

PHASE OF DEVELOPMENT: Phase 1 followed by randomized Phase 2

### INVESTIGATORS AND TRIAL CENTERS: Multicenter

### OBJECTIVES:

### Primary:

To determine the safety and tolerability of Seneca Valley Virus (SVV)-001 when administered intratumorally as single or multiple doses in combination with nivolumab in patients with poorly differentiated neuroendocrine carcinomas (NECs) and well-differentiated neuroendocrine tumors (NETs).

### Secondary:

To evaluate median progression-free survival with SVV-001 in combination with nivolumab in patients with poorly differentiated NECs and well-differentiated NETs (in dose-escalation phase of Part I).

To evaluate the immunogenicity of SVV-001.

### INVESTIGATIONAL MEDICINAL PRODUCT, DOSE, AND MODE OF ADMINISTRATION:

Investigational medicinal product (IMP): SVV-001 Drug Product and nivolumab.

SVV-001 is a live virus manufactured from a master cell bank in PER.C6® cells. Manufacturing of SVV-001 was originally performed by Molecular Medicine BioServices, (MMB) Inc., San Diego, CA. The Drug Product to be used in this trial was made by the Clinical Vector Core at the Children's Hospital of Philadelphia by diluting the original clinical material made at MMB 30-fold in aqueous buffer composed of 200 mM Tris, 50 mM HEPES, and 10% glycerol, pH 8 then vialing in 1.5 mL cryovials with approximately 0.35 mL/vial with the concentration to be determined and stored at <−60° C.

Immediately prior to use, SVV-001 will be thawed and diluted to 4 mL with 0.9% NaCl Injection USP, according to instructions in the Pharmacy Manual. The virus must be used within 6 hours of dilution and administered via intratumoral injection.

The virus doses 2.2×10⁸, 2.2×10⁹, and 2.2×10¹⁰ viral genomes (VG) per patient-day will be diluted in 0.9% NaCl Injection USP to achieve a final injected volume of 4 mL.

Nivolumab will be provided in commercial packaging with additional labeling for use in this trial, as needed to fulfill regulatory requirements. Complete preparation, administration, and storage instructions for nivolumab are provided in the Prescribing Information.

### TRIAL DESIGN AND METHODOLOGY:

This is a Phase 1 dose-escalation and cohort-expansion trial of SVV-001 administered intratumorally in combination with the immune checkpoint blocker (ICB) nivolumab in patients with poorly differentiated NECs and well-differentiated NETs.

This trial is designed to establish the maximum tolerated dose (MTD) and recommended Phase 2 dose (RP2D) of single and multiple doses of SVV-001 in combination with nivolumab.

Between 21-36 eligible patients with poorly differentiated NECs and well differentiated NETs will be enrolled in Part I. All patients are required to be positive for favorable SVV-001 biomarker mRNA expression profile using a nanostring RT-qPCR platform, as described in Example 5. Only patients predicted to be sensitive to SVV-001 will be included in the trial.

### MAJOR ELIGIBILITY CRITERIA:

The patient population will include male and female patients, 18 years or older, who are SVV-001 biomarker panel-favorable using a biomarker gene expression panel, which identifies patients likely to respond to SVV-001. The biomarker panel will be performed using a RT-qPCR platform in a Clinical Laboratory Improvement Amendments-approved laboratory, as described in Example 5, RNAseq, and/or by using an nCounter® platform for the panel. Pre-treatment biomarker analyses will be performed with tumor tissue (paraffin block [preferred] or frozen tissue) from either primary or recurrent tumor, although samples from recurrence are preferred if both are available. If archived tissue is not available, a fresh tumor biopsy specimen may be used. Patients will have either of the following neoplasm types: 1) Part I, Group 1: histologically or cytologically confirmed diagnosis of well-differentiated Grade 1 and Grade 2 NET or 2) Part I, Group 2: histologically or cytologically confirmed diagnosis of poorly differentiated NEC (large cell neuroendocrine carcinoma, extra thoracic small cell carcinoma, poorly differentiated neuroendocrine carcinoma). Patients will have a life expectancy of 6 months or greater as assessed by the treating oncologist and have advanced metastatic disease that has progressed on at least 2 lines of available therapy (prior progression on 1 line of therapy for NEC cohort permitted) and for whom no curative treatment is available. All patients in Part IB will have 1) measurable disease as determined by Response Evaluation Criteria in Solid Tumors (RECIST) v1.1; 2) at least one lesion suitable for multiple injections (nominally 6 injections every 2 weeks) with SVV-001, and 3) lesions for injection must be ≥10 mm and ≤50 mm in longest diameter and deemed safe and suitable for injection by the investigator. Patients in Part IA need to have histological evidence of NEC or NET with radiological confirmation.

### PLANNED SAMPLE SIZE:

Between 21-36 patients will be enrolled in the 3+3 dose-escalation portion of the trial (Part I).

### TRIAL TESTS AND OBSERVATIONS:

The primary safety endpoints are as follows:
Frequency and severity of AEs and serious AEs (SAEs) graded according to National Cancer Institute Common Terminology Criteria for Adverse Events (NCI CTCAE) version 5.0
Incidence of DLTs (Part I only)
Change in clinical laboratory parameters, vital signs, electrocardiograms, and Eastern Cooperative Oncology Group performance status

Secondary endpoints are as follows:
Overall Response Rate (ORR) according to RECIST 1.1 and or iRECIST
PFS at 6 and 12 months according to RECIST 1.1 and or iRECIST
Duration of response
Clinical benefit rate (complete response [CR]+partial response [PR]+stable disease for ≥6 months)
Percentage of patients with total antibodies and neutralizing antibodies against SVV-001
Kinetics of appearance of neutralizing antibodies
Levels of antibodies
Antibody subtypes (IgM, IgG)
Time from first dose of trial medication to date of death due to any cause.

### STATISTICAL METHODS:

### Clinical Activity Analyses:

PFS is defined as the time from first dose of SVV-001 until radiographically determined disease progression or death due to any cause, whichever occurs first. Patients who are still alive or who have no progressive disease reported at the 3-year time point, will be censored at their last evaluable tumor assessment.

Overall survival is defined as the time from first dose of trial medication (SVV-001) to date of death due to any cause.

Percent change from baseline in sum of diameters for injected and non-injected lesions will be evaluated according to iRECIST.

ORR is defined as the percentage of patients with confirmed PR or CR based on tumor assessment as determined by RECIST 1.1. Patients who discontinue due to toxicity or confirmed clinical progression prior to post baseline tumor assessments will be considered as nonresponders.

Clinical benefit rate is defined as the percentage of patients with confirmed CR, PR, or stable disease. Patients who discontinue prior to post baseline tumor assessments will be considered as nonresponders.

Duration of response is defined as time from date of reported confirmed PR or CR to the date of progression and will be summarized descriptively using summary statistics. Additionally, a listing of duration of response for those patients experiencing response will be provided.

### Pharmacodynamic and Biomarker Analyses:

Descriptive statistics will be used to evaluate the mean, median, absolute number, and percent change from baseline for immunohistochemistry, blood tumor markers, and soluble biomarkers. Exploratory analyses may be performed to evaluate a possible correlation between each of these endpoints and disease response and/or toxicities. Additional analyses may also be performed.

### Safety Analyses:

Adverse events will be coded according to Medical Dictionary for Regulatory Activities (MedDRA) version 21.0 or higher and assessed for severity using NCI-CTCAE version 5.0. Adverse events including immune-related adverse events will be summarized by system organ class and preferred term and presented in decreasing order of incidence. Dose-limiting toxicities will also be summarized by dose and cohort. The incidence of treatment-emergent AEs (events with onset dates on or after the start of the SVV-001) will be included in incidence tables. Events with missing onset dates will be included as treatment emergent. If a patient experiences more than 1 occurrence of the same AE, the occurrence with the greatest severity and the closest association with the IMP will be used in the summary tables. Serious adverse events and AEs causing discontinuation will be tabulated. All AEs will be listed by patient, along with information regarding onset, duration, relationship and severity to IMP, action taken with IMP, treatment of event, and outcome.

Vital sign measurements, electrocardiogram data, clinical laboratory assessments, and ECOG performance status at baseline and during the trial and any changes from baseline will be examined. Treatment-emergent changes in key laboratory parameters will be identified. Clinical laboratory data will be summarized for each time point that specimens are collected. Changes from baseline for clinical laboratory values may also be explored as specified in the SAP.

Summary tables will be provided for concomitant medications initiated prior to trial enrollment or during the trial period.

### Definitions of Abbreviations and Terms

| Abbreviation | |
|---|---|
| or Term | Definition |
| AchEA | cetylcholine esterase |
| ADL | Activities of daily living |
| AE | adverse event |
| ALP | Alkaline phosphatase test |
| ALT | alanine aminotransferase |
| ANTXR1 | anthrax toxin receptor 1 |
| AST | aspartate aminotransferase |
| BUN | Blood urea nitrogen |
| CFR | Code of Federal Regulations |
| CLIA | Clinical Laboratory Improvement Amendments |
| CR | complete response |
| CT | computerized tomography |
| CTC | Common Toxicity Criteria |
| CTCAE | Common Terminology Criteria for Adverse Events |
| CTLA-4 | cytotoxic T lymphocyte antigen 4 |
| CTCAE | Common Terminology Criteria for Adverse Events |
| DLT | dose-limiting toxicity |
| dMMR | deficiency in mismatch repair |
| ECG | electrocardiogram |
| ECOG | Eastern Cooperative Oncology Group |
| eCRF | electronic CRF |
| FACS | fluorescence-activated cell sorting |
| FDA | Food and Drug Administration |
| GCP | Good Clinical Practice |
| HIV | human immunodeficiency virus |
| ICB | immune checkpoint blocker |
| ICF | Informed Consent Form |
| ICH | International Conference on Harmonization |
| IFN | Interferon |
| Ig | Immunoglobulin |
| IHC | immunohistochemistry |
| IL | Interleukin |
| ILD | Interstitial disease |
| IMAR | Immune-mediated adverse reactions |
| irAE | Immune-related adverse event |
| IMP | investigational medicinal product |
| IRB | Institutional Review Board |
| iRECIST | immune-related Response Evaluation |
| | Criteria in Solid Tumors |
| IV | intravenous(ly) |
| LFT | Liver function tests |
| LLN | Lower limit of normal |
| MedDRA | Medical Dictionary for Regulatory Activities |
| MRI | magnetic resonance imaging |
| MSI | microsatellite instability |
| MTD | maximum tolerated dose |
| NCCN | National Comprehensive Cancer Network |
| NCI | National Cancer Institute |
| NEC | neuroendocrine carcinomas |
| NET | neuroendocrine tumor |
| Nivo | Nivolumab |
| ORR | overall response rate |
| PD-1 | programmed death receptor-1 |
| PD-L1 | programmed death-ligand |
| PET | positron emission tomography |
| PFS | progression-free survival |
| PJP | Pneumocystis jirovecii pneumonia (formerly |
| | known as Pneumocystis carinii pneumonia) |
| PR | partial response |
| Q | Every |
| RECIST | Response Evaluation Criteria in Solid Tumors |
| RNA | ribonucleic acid |
| RP2D | recommended phase 2 dose |
| SAE | serious adverse event |
| SAP | statistical analysis plan |
| SCLC | small-cell lung cancer |
| SRC | Safety Review Committee |
| SVV-001 | Seneca Valley Virus |
| T3 | Triiodothyronine |
| T4 | Thyroxine |
| TB | Total bilirubin |
| TEAE | treatment-emergent adverse event |
| TEM8 | Tumor Endothelial Marker 8 |
| TCID | tissue culture infective dose |
| TMB | tumor mutational burden |
| TNF | Tumor necrosis factor |
| TSH | Thyroid-stimulating hormone |
| ULN | upper limit of normal |
| US | United States |
| VG | viral genomes |
| VP | viral particle |
| Wk | Week |

### Objectives and Endpoints

TABLE 26
Objective and Endpoints

| Part I | |
|---|---|
| Objectives | Endpoints |
| Primary (Safety) | |
| To determine the safety and tolerability of | Frequency and severity of AEs graded |
| SVV-001 when administered | according to NCI CTCAE version 5.0 |
| intratumorally assingle or multiple doses, | Incidence of DLTs (Part I only) |
| in combination with nivolumab in patients | Change in clinical laboratory parameters, |
| with poorly differentiated NECs and well- | vitalsigns, electrocardiograms, and ECOG |
| differentiated NETs | performance status |
| Secondary | |
| To evaluate median progression-free | Overall response rate (ORR) according |
| survival with SVV-001 in combination | to RECIST 1.1 (Part I) and or iRECIST |
| with nivolumabin patients with poorly | (PartI) |
| differentiated NECs andwell- | Progression-free survival (PFS) at 6 and |
| differentiated NETs | 12 months according to RECIST 1.1 |
| | and or iRECIST |
| | Duration of response |
| | Clinical benefit rate (CR + PR + |
| | stable disease for ≥6 months) |
| To evaluate the immunogenicity of SVV- | Percentage of patients with total |
| 001 | antibodies and neutralizing antibodies |
| | against SVV-001 |
| | Kinetics of appearance of neutralizing |
| | antibodies |
| | Levels of antibodies |
| | Antibody subtypes (IgM, IgG) |
| Exploratory Analyses | |
| To evaluate the kinetics of viral release, | Kinetics and levels of SVV-001 viral RNA |
| shedding, and viral load in plasma | in blood (ie, viral RNA load) and stool |
| To characterize the impact of SVV-001 | Difference between pre- and post- |
| on expression of soluble cytokines | dose soluble cytokines in plasma |
| To characterize changes in the | Evaluation of immune-cell infiltration |
| immune environment (Part I only; | and levels of SVV-001 replication in |
| Cohorts 6) | injected tumors |
| | Quantification of expression of PD-1, PD- |
| | L1, and other immune checkpoint |
| | receptor/ligand pairs on tumor cells and |
| | immune cells in injected tumors |
| | Quantification of expression of TEM8 and |
| | Type 1 IFN response genes in injected |
| | tumors |
| | Profiling of injected tumor immune |
| | responseand inflammation by RT-PCR |
| | and/or |
| | Nanostring IO360 panel mRNA analysis |
| | Evaluation of the diversity and clonality of |
| | the tumor infiltrating T-cell receptor |
| | repertoire in injected tumors pre- and post- |
| | treatment by analysis of V-beta sequences |
| | Evaluation of the baseline tumor |
| | genomic profile (tumor suppressor |
| | mutations), TMB, and DNA repair |
| | competence (MSI/dMMR) |
| | Changes in circulating immune cell |
| | phenotypes and activation markers |
| | measuredusing flow cytometry |
| To evaluate the immunogenicity of SVV- | Changes in circulating immune cell |
| 001 | phenotypes and activation markers |
| | measuredusing flow cytometry |

Abbreviations: AE = adverse event; CR = complete response; dMMR = deficiency in mismatch repair; ECOG = Eastern Cooperative Oncology Group; IFN = interferon; Ig = immunoglobulin; iRECIST = immune-related Response Evaluation in Solid Tumors; MSI = microsatellite instability; NCI-CTCAE = National Cancer Institute Common Terminology Criteria for Adverse Events; NEC = neuroendocrine carcinoma; NET = neuroendocrine tumor; PD-1 = programmed death receptor-1; PD-L1 = programmed death-ligand 1; PR = partial response; RECIST = Response Evaluation in Solid Tumors; TEM8 = Tumor Endothelial Marker 8; TMB = tumor mutational burden.

### Part I—Dose Escalation

### Overview

Between 21-36 eligible patients with poorly differentiated NECs and well-differentiated NETs will be enrolled in Part I. All patients are required to be positive for favorable SVV-001 biomarker profile, defined using a panel of genes that identify sensitivity versus resistance to SVV-001 cytotoxicity. This panel incorporates expression of the SVV-001 receptor, TEM8, a known prerequisite for SVV-001 infection in addition to genes involved in immune activation, mRNA translation, and cell adhesion. The biomarker panel assay will be conducted in an established Clinical Laboratory Improvement Amendments (CLIA) laboratory (see Example 5).

In Part IA (evaluation of single escalating doses of SVV-001), up to 3 cohorts will be enrolled sequentially and assessed using 3+3 dose escalation to evaluate the safety and tolerability and identify the MTD and RP2D for treatment with single doses of SVV-001 in combination with standard dose of Nivolumab in Part IA of the trial as follows. To protect subject safety, enrollment will be staggered for the first 2 subjects in each dose cohort by at least 2 weeks:
Cohort 1: SVV-001 at 2.2×10⁸ VG (×1 dose on Day 1)
Cohort 2: SVV-001 at 2.2×10⁹ VG (×1 dose on Day 1)
Cohort 3: SVV-001 at 2.2×10¹⁰ VG (×1 dose on Day 1).

Patients will be observed for 2 weeks for dose-limiting toxicities (DLTs) at each dose level before proceeding with the next higher single-dose cohort.

If no safety issues are identified in single-dose cohorts, Part IB (evaluation of multiple escalating doses [6 doses each 2 weeks apart] of SVV-001) of the trial will commence.

Up to 2 cohorts will be enrolled sequentially and assessed using 3+3 dose escalation to identify the MTD and RP2D for treatment with multiple doses of SVV-001 in combination with standard dose of Nivolumab in Part IB of the trial as follows:
Cohort 4: SVV-001 at 2.2×10⁹ VG per day (i.e., per administration)×6 doses
Cohort 5: SVV-001 at 2.2×10¹⁰ VG per day (i.e., per administration)×6 doses

The starting dose for Cohort 4 will be 1 dose lower than the MTD/RP2D identified for single dose SVV-001 from Cohorts 1-3 (ie, if all 3 cohorts complete with no DLTs, then the starting dose forCohort 4 would be 2.2×10⁹ VG per day (i.e., per administration))

Following identification of the MTD/RP2D for SVV-001 multiple dosing treatment, an additional cohort (Cohort 6) will be added, where 10 patients will be treated with SVV-001 at MTD/R2PD in combination with standard dose of Nivolumab and 1 injected lesion per patient will be biopsied at Day 15 (after 1 SVV-001 injection, n=3 patients) or Day 29 (after 2 SVV-001 injections, n=3 patients).
Cohort 6: SVV-001 at MTD/RP2D dose per day (i.e., per administration)—biopsied on Day 15 or Day 29.

A Data Safety Monitoring Board (DSMB) will review the safety data (adverse events [AEs] and laboratory toxicities) of each lower-dose cohort before proceeding to the next dose level.

A patient who withdraws from the trial within the DLT evaluation period for reasons other than drug-related AE will be replaced.

Escalation to higher-dose (SVV-001) cohorts is not permitted during the trial. After a DLT is experienced by a patient, dose interruption, modifications, or dose delays may apply, as per the investigator's judgement.

### Parts IA and IB

Patients will be treated with intratumoral injections of SVV-001. Note that the doses represent the total dose a patient will receive that day, not the dose per lesion.

SVV-001 will be administered intratumorally as a single dose (Part IA; Cohorts 1-3) or biweekly for 6 doses (ie, for 3 months; Part IB; Cohorts 4-6). Three patients will be enrolled in Cohort 1 and receive 1 dose of SVV-001 at 2.2×10⁸ VG. If none of the 3 patients experiences a DLT during a 4-week observation period, the next dose cohort of 3 patients (Cohort 2) will be enrolled at the next higher dose level (2.2×10⁹ VG). If 1 of 3 patients in Cohort 1 experiences a DLT, Cohort Iwill be expanded to add 3 additional patients. If only 1 of these 6 patients in Cohort 1 has a DLT, then 3 patients will be enrolled into Cohort 2 at 2.2×10⁹ VG. If 2 or more subjects of the 3 to 6 patients tested at the lowest-dose cohort (ie, Cohort 1; 2.2×10⁸ VG) experience a DLT, then newpatients will be enrolled at the 2.2×10⁷ VG dose level until that cohort has 6 patients with no more than 1 patient experiencing a DLT. If 2 or more patients experience a DLT at the 2.2×10⁷ VG dose level, new patients will be enrolled at the 2.2×10⁶ VG dose level until that cohort has 6patients with no more than 1 patient experiencing a DLT. If 2 or more patients experience a DLTat the 2.2×10⁶ VG dose level, the trial will be terminated. The dose level at which no more than 1 patient experiences a DLT will be designated the MTD for the single dose SVV-001.

If an MTD is not established based on the criteria, then the highest dose (2.2×10¹⁰ VG) will be considered the MTD for the cohorts in Part IA of the trial.

Part IB will commence at 1 dose level less than the MTD established in Part IA of the trial. Dosing will proceed using the same rules as described for Part IA of the trial. Note that the doses of SVV-001 represent the total dose a patient will receive that day, not the dose per lesion.

Patients in Part IB will receive treatment with SVV-001 for 12 weeks (6 total doses, one every 2 weeks).

Intravenous (IV) doses of nivolumab will remain constant in all dose cohorts. The nivolumab dose will be 240 mg administered intravenously (IV) every 2 weeks during SVV treatment. Following that, patients will continue on 480 mg nivolumab IV every 4 weeks until progression, end of treatment, or unacceptable toxicity. The observation period for Part IB will be 4 weeks. Three patients will be enrolled and dosed in Cohort 4. If none of the 3 patients experiences a DLT during the 4-week observation cycle, the next dose cohort of 3 patients will be enrolled at the next higher dose level. If 1 of 3 patients in the initial dose Cohort 5 experiences a DLT, then Cohort 5 will be expanded to 6 patients. If only 1 or less of these 6 patients has a DLT, then it will be considered MTD.

### Duration of Trial

The trial will include a screening period (up to 28 days), a treatment period, and a follow-up period. The maintenance period will be administering 480 mg nivolumab IV every 4 weeks until progression, end of treatment, or unacceptable toxicity.

Patients will receive treatment with single dose SVV-001 or multiple dose SVV-001 for 12 weeks (doses are every 2 weeks for 6 total) with nivolumab. The nivolumab dose will be 240 mg administered intravenously (IV) every 2 weeks during SVV treatment. Following that, patients will continue with 480 mg nivolumab IV every 4 weeks until progression, end of treatment, or unacceptable toxicity.

The safety follow-up visit will be conducted 28 days after the last dose of trial treatment (SVV-001 or nivolumab).

### Definition of Dose-Limiting Toxicities

The DLT evaluation period is 4 weeks for dose-escalation parts (Parts IA and IB). The DLTs are defined as any of the following that occur during the DLT evaluation period, graded according to the National Cancer Institute's Common Terminology Criteria for Adverse Events (NCI-CTCAE), version 5.0. All AEs, unless they have been clearly determined by the investigator to be not related to treatment, will be included in the evaluation of safety, DLTs, and the RP2D. No DLT evaluation will be done in Cohort 6 enrollment since it is an exploratory biopsy cohort.

Nonhematological DLTs are defined as follows:

Any ≥Grade 3 events assessed as related to the investigational medicinal product (IMP) or injection, with the exception of the following:

Symptoms such as nausea and vomiting, injection site pain, fever, chills, which are <Grade 4 that resolve within 72 hours after treatment with standard care; Asymptomatic changes in electrolytes or liver function tests that resolve within 48 hours;
Tumor pain that improves following treatment with any analgesic.

Hematological DLTs are defined as follows:
≥Grade 4 neutropenia of any duration or Grade 3 neutropenia that lasts more than 7 days
Febrile neutropenia
Grade 3 or 4 thrombocytopenia in the presence of Grade 2 bleeding
≥Grade 3 anemia.

### TRIAL POPULATION

### Inclusion Criteria

Patients must meet all of the following criteria in order to be included in the trial:
1. Male or female patients, 18 years of age or older at the time of consent.
2. A favorable SVV-001 biomarker profile defined using a panel of genes which identify sensitivity versus resistance to SVV-001 cytotoxicity using the nCounter® platform performed in a CLIA-approved laboratory.
3. Life expectancy of 6 months or greater as assessed by the treating oncologist.
4. Have advanced metastatic disease that has progressed on at least 2 lines of available therapy (prior progression on 1 line of therapy for NEC cohort permitted) and for whom no curative treatment is available.
5. Histologically or cytologically confirmed diagnosis of well differentiated neuroendocrine tumor (NET) or poorly differentiated NEC (large-cell neuroendocrine carcinoma, extra thoracic small-cell carcinoma, poorly differentiated neuroendocrine carcinoma).
6. Parts IB and II only: Measurable disease as determined by Response Evaluation Criteria inSolid Tumors (RECIST) v1.1. At least one lesion must be suitable for multiple injections (nominally 6 injections every 2 weeks) with SVV-001. Lesions for injection must be ≥10 mm and ≤50 mm in longest diameter and deemed safe and suitable for injection by the investigator.
   For patients in Part IA, in addition to histological or cytological confirmation of NEC orNET (see Inclusion #5), radiological confirmation of tumor is required.
7. Eastern Cooperative Oncology Group (ECOG) performance status of 0 or 1 (Table 31).
8. Recovery to Grade 1 or baseline of any clinically significant toxicity of prior treatments (excluding alopecia) prior to initiation of IMP administration.
9. Adequate hematological, renal, and liver function defined as follows:
a. Hepatic:
   i. Serum alanine aminotransferase (ALT) and aspartate aminotransferase (AST) ≤2.5×upper limit of normal (ULN) (≤5×ULN if liver metastasesare present)
   ii. Serum bilirubin ≤1.5×ULN (unless due to Gilbert's syndrome orhemolysis)
b. Renal:
   i. Creatinine clearance ≥50 mL/minute using Cockcroft Gault equation
c. Hematologic:
   i. Absolute neutrophil count ≥1500/μL
   ii. Platelet count ≥100,000/μL
   iii. Hemoglobin ≥9.0 g/dL
   iv. International normalization ratio within the institutional normal range
   v. Normal prothrombin time and partial thromboplastin time
10. For Cohort 6 patients only, patients must have at least 2 sites of disease suitable for biopsy and be willing and able to undergo required tumor biopsies according to the treating institution's guidelines, including 1 pretreatment and 1 post-treatment tumor biopsy (total of 2 biopsies).
11. Women of childbearing potential must agree to use a reliable form of contraceptive during the trial treatment period and for at least 7 months following the last dose of IMP.
12. Male patients must agree to use an adequate method of contraception during the trial treatment period and for at least 7 months following the last dose of IMP.
13. Patient is willing and able to comply with all protocol-required assessments, visits, and procedures.
14. Provide written informed consent prior to performing any trial-related procedure.

### Exclusion Criteria

Patients meeting any of the following criteria are ineligible to participate in this trial:
1. Any active second malignancy within the 3 years prior to the screening visit, unless the patient has undergone curative surgery for the tumors such as in situ cervical cancer or squamous cell cancer of the skin; discussion and approval with the sponsor/medical monitor is required before enrolling subject.
2. Less than 3 weeks from prior cytotoxic chemotherapy or radiation therapy; and less than 5 half-lives or 6 weeks, whichever is shorter, from prior biologic therapies, prior to the first dose of SVV-001.
3. Has undergone a major surgical procedure (as defined by the investigator) or significant traumatic injury within 28 days prior to the first dose of SVV-001.
4. Requires transfusion support or treatment with growth factors in order to maintain adequate blood counts.
5. Has any physical abnormality of the tissue/organ to be biopsied that would put the patient at increased risk of bleeding secondary to the injection and/or biopsy.
6. Has received a live-virus immunization within 30 days prior to the screening visit or anticipates receiving a live virus immunization during the trial or within 30 days of the last treatment with IMP.
7. Presence of an active autoimmune or inflammatory disease requiring systemic treatment within the past 2 months or a documented history of clinically severe autoimmune disease that requires systemic steroids or other immunosuppressive medications. Local steroid injections, intermittent use of topical, inhaled, ophthalmologic, intra-articular, topical, or intranasal corticosteroids, or systemic corticosteroids at physiologic doses not to exceed 10 mg/day of prednisone or equivalent would not result in exclusion from the trial.
8. Presence of primary immunodeficiency or receiving systemic steroids of >10 mg/day prednisone or equivalent or other immunosuppressive agents within 14 days prior to the first dose of SVV-001.
9. Any active infection, including known infection with human immunodeficiency virus (HIV), active hepatitis, or seropositive for hepatis B immunoglobulin (Ig)M coreantibody or hepatitis C RNA at the screening visit.
10. Patients with a history of solid-organ or bone-marrow transplant.
11. Known hypersensitivity to SVV-001 or its excipients.
12. Known hypersensitivity to ipilimumab or nivolumab or their excipients (applicable to patients in combination cohorts (Part IB and in Part II only).
13. Has known untreated central nervous system metastases. Patients with treated brain metastases are eligible as long as they are stable and there is no evidence of progression for at least 4 weeks after central nervous system-directed treatment, as ascertained by clinical examination and brain imaging (magnetic resonance imaging [MRI] or computed tomography [CT]) during the screening period.
14. Any clinically significant (ie, active) cardiovascular disease, including cerebral vascularaccident/stroke (<6 months prior to enrollment), myocardial infarction (<6 months priorto enrollment), unstable angina, congestive heart failure (>New York Heart Association Classification Class II), or serious cardiac arrhythmia requiring medication.
15. Any chronic illness, psychiatric condition, or social situation that is life threatening or, in the opinion of the investigator, renders the patient unsuitable for participation in a clinical trial due to possible noncompliance or would place the patient at an unacceptable risk and/or have the potential to affect interpretation of the results of the trial.
16. Female subjects who are breastfeeding and/or who have a positive pregnancy test result prior to receiving any treatment with IMP.
17. Has received treatment with another ICB.
18. Is receiving any prohibited medication or would be likely to require prohibited concomitant therapy during the trial.

### Treatments

### Treatments Administered

### Investigational Product—SVV-001

### Administration

SVV-001 will be administered intratumorally as a single dose or as multiple doses on Days 1, 15, 29, 43, 57, and 61.

Up to 10 target lesions can be injected, with a maximum of 2 target lesions per organ. The virus doses 2.2×10⁸, 2.2×10⁹, and 2.2×10¹⁰ VG per patient per day will be diluted in 0.9% NaCl Injection USP to achieve a final injected volume of 4 mL. To deliver the 2.2×10¹⁰ VG dose, 0.35 mL virus is mixed with 3.65 mL saline and the entire 4 mL injected intratumorally into the patient's tumors per patient day. The 2.2×10⁹ VG dose is achieved by mixing 0.35 mL virus with 39.65 mL saline and using a total of 4 mL per intratumoral administration. The 2.2×10⁸ VG dose is achieved by first mixing 0.35 mL virus with 399.65 mL saline and using a total of 4 mL per intratumoral administration.

Lesions will be injected based upon their maximum lesion diameter with a total delivered volume of 4 mL per patient per day. Lesions between 1 and 5 cm in their longest diameter are eligible for injection and defined as target lesions. The largest target lesions (4 or 5 cm in diameter) will be injected with 4 mL SVV-001. Target lesions of 3 cm diameter will be injected with 3 mL virus; 2 cm target lesions will be injected with 2 mL virus and 1 cm target lesions will be injected with 1 mL virus. The largest target lesions should be injected first using the volumes above until all 4 mL of the drug is injected. For example, if there is only 1 injectable 5 cm target lesion, the entire 4 mL drug dose will be injected into this target lesion. If we have a patient with two 2 cm target lesions and two 1 cm lesions, the two 2 cm target lesions will be injected with 2 mL each and the remaining 1 cm lesions will not be injected.

Injectable lesions should be devoid of major neuro-vascular structure (e.g., carotid artery) or other key anatomical structure (e.g., pulmonary airway) to minimize the potential adverse effects of post-treatment tumor swelling or necrosis on these structures. All the visceral injections will be done under image guidance.

Patients in Cohorts 6 must have at least 2 lesions available for biopsy (pre- and post-treatment). The lesions assigned for biopsy in Cohorts 6 will not be defined as target lesions. If patients in Cohorts 6 have ≥3 injectable lesions, then the non-biopsied lesions may be defined as target lesions.

### Nivolumab

For Part I, the nivolumab dose will be 240 mg administered IV every 2 weeks during SVV treatment followed by once every 4 weeks. Nivolumab will start on Day 15, 2 weeks after the first dose of SVV-001.

Complete preparation, administration, and storage instructions for nivolumab are provided in the Prescribing Information.

### Trial Assessments

### Screening/Baseline Assessments

Only patients who meet all inclusion and exclusion criteria specified will be enrolled in this trial.

Absence of active central nervous system tumor or metastases will be confirmed by MRI of the brain (or CT with intravenous contrast for subjects who cannot tolerate an MRI).

Demographics include age, sex, race, ethnicity, smoking history, and smoking status.

Subject medical and surgical history includes a thorough review of previous cancer therapies, cancer history, surgeries, and past and ongoing concomitant illnesses.

Malignancy history should include tumor type, stage, sites of metastases, prior therapies (including dates of treatment and reasons for treatment discontinuation, responses and dates of progression, as available and applicable) and mutational status (including presence of specific driver mutations and their molecular characterization, as available and applicable).

### Clinical Activity Profile Assessments

Treatment response will be assessed by radiographic-tumor evaluation at protocol-specified time points. Diagnostic quality, spiral CT scans are recommended; other methods may be used if performed consistently throughout the trial for each individual subject. Scans of the chest, abdomen, and pelvis must be obtained; scans of the neck must also be obtained if there is documented or suspected involvement in this region. Positron emission tomography (PET) may be used in an ancillary manner; however, no decisions relating to progressive disease may be made based on PET alone. Clinical response will be determined by the investigator at each assessment according to RECIST 1.1 and iRECIST. A confirmed tumor response is defined as CR, PR, or stable disease per iRECIST noted on 2 consecutive evaluations completed at least 6 weeks apart.

Patients treated with immunotherapies often exhibit tumor “pseudo-progression” where the tumor increases in size transiently due to tumor inflammation. For the subjects who continue SVV-001 or nivolumab treatment beyond progression, further progression is defined as an additional 20% increase in tumor burden from time of initial progression. This includes an increase in the sum of diameters of all target lesions and/or the diameters of new measurable lesions compared with the time of initial progression. SVV-001 and nivolumab treatment should be discontinued permanently upon documentation of further progression.

### Investigator's Assessment of Response to Treatment by Immune Response

Subjects treated with SVV-001 plus nivolumab will be permitted to continue the same treatment beyond initial RECIST v1.1 defined progressive disease, as long as the following criteria are met:
Subjects who will be treated beyond disease progression must provide informed consent before continuing study drug
The subject demonstrates investigator-assessed clinical benefit, and does not have rapid disease progression
Tolerance of study drug
Stable performance status.
Treatment beyond PD will not delay an imminent intervention to prevent serious complications of PD (eg: CNS mets).

### Viral Burden and Viral Shedding

Blood samples for analysis of viral-burden will be collected at various time points throughout the trial as indicated in Table 30. Sensitive, qualified assays will be used to measure blood concentrations of SVV-001. Virus shedding will be evaluated from stool obtained at various time points throughout Part IA and IB of the trial as indicated in Table 30.

### Part 1 Viral Load

The samples collected and analyzed is serum. Virus detection is performed by a qRT-PCR assay and, if positive, a cell-based infectivity (TCID50) assay is performed. Monitoring serum compartments are required for the first two weeks. Clearance for a compartment is defined as two consecutive samples without detectable virus by both assays and without re-emergence, if during the first two weeks. If a compartment is still positive for virus at the end of 2 weeks, only the positive compartment(s) and serum will be collected. These compartments are tested by both assays until all are negative for 2 consecutive points.

### Virus Neutralization

For neutralization, the pre-dose serum sample and subsequent serum samples will be tested until end of collections using the serum drawn for above. Analysis has been done after all sampling is completed for the patient.

### Shedding Plan

Sampling will start on day 1, 3, 7, 10 and then weekly. Virus shedding will be assessed by sampling stool (using a fecal swab) taken at indicated time points utilizing a qRT-PCR assay. Shedding will be evaluated in patients treated with SVV-001 (Phase 1 only) and reported to FDA. If positive by PCR, the TCID50 endpoint dilution assay will be performed, and patients' stool will be collected every week (+/−1 day) after the last dose until it's negative.

### Pharmacodynamic and Biomarker Assessments

Prior to enrollment, patients will be selected to be SVV-001 biomarker panel-favorable using a biomarker gene expression panel that identifies patients likely to respond to SVV-001. The SVV-001 biomarker panel will be performed on a RT-qPCR platform in a CLIA-approved laboratory (see Example 5), RNAseq, and/or nCounter®

Blood and/or tissue samples (post-treatment tumor biopsies obtained from Cohorts 6 only) for pharmacodynamics and biomarker testing will be collected at the time points throughout the trial as indicated in Table 30. Samples may be retained for additional testing as needed. Because some tissue samples may be limiting, comprehensive analysis of all biomarkers may not be feasible and thus sample analysis will be prioritized.

A summary of planned tests using blood, serum, stool, and tumor samples is provided in Table 27. Samples will be obtained following the schedule in Table 30. Serum virus titer and fecal virus shedding will be evaluated for patients in Part IA as part of the safety analysis. The other assays, except FACS analysis, can be stored and batch-tested FACS analysis will be performed on the same day as blood collection.

TABLE 27
Biomarker Test and Analyses

| | Tumor Biopsy | Peripheral Blood | | |
|---|---|---|---|---|
| Sample | Tissue* | Mononuclear Cells | Blood Plasma | Fecal Swab |
| Tests | | | | |
| | Genomic DNA, | FACS: T-cell receptor | Cytokines: IL-2, | |
| | mutationalload, | V- beta changes, immune | IL-4, IL-6, IL-10, | |
| | MSI/dMMR (only | cell markers: CD3, CD8, | IFN-γ, TNF-α, | |
| | pretreatment | CD4, FoxP3, CD56, B220, | GM-CSF, CXCL8 | |
| | sample) | T cell activation | (IL-8), | |
| | | markers: PD-1, TIM-3, | CXCL9 (Mig-1), | |
| | | 4-1-BB, Ki-67 | CXCL10 (IP-10), | |
| | | | CXCL11 (I-Tac), | |
| | | | CCL3 (MIP-1a) | |
| | SVV-001 | | | |
| | Biomarker gene | | | |
| | expression panel | | | |
| | using RT-qPCR | | | |
| | platform (pre- | | | |
| | treatment only) | | | |
| | IHC - SVV-001 | Freeze sample for | Test for anti-SVV- | |
| | replication in | monoclonal | 001 and | |
| | tumor | antibody analysis | neutralizing | |
| | | | antibodies | |
| | IHC - immune | | Test for | Test for |
| | markers on tumor | | circulating SVV- | shed |
| | cells: PDL-1, Ki- | | 001 virus levels | virus |
| | 67, CD31, | | | |
| | chromogranin A | | | |
| | IHC- Immune cell | | | |
| | infiltration: CD3, | | | |
| | CD8, CD4, FoxP3, | | | |
| | CD56, B cell | | | |
| | (B220), macrophage | | | |
| | (CD163), MDSC, | | | |
| | PD-1, CXCR3 | | | |
| | Nanostring - | | | |
| | mRNA gene | | | |
| | expression: T-cell | | | |
| | receptor V-beta | | | |
| | changes, IFN- | | | |
| | gamma signature, | | | |
| | “hot” versus | | | |
| | “cold” tumor | | | |
| | detection | | | |
| | (Nanostring IO360 | | | |
| | panel) comparing | | | |
| | pre- versus post- | | | |
| | treatment samples | | | |

Abbreviations: dMMR = deficiency in mismatch repair; FACS = fluorescence-activated cell sorting; IHC = immunohistochemistry; MSI = microsatellite instability; PD-1 - programmed death receptor-1; PD-L1 = programmed death-ligand 1.
* Formalin-fixed paraffin embedded tissue block sample.

### Tumor Biopsy Tissue

Archival tissue specimens will be screened using our RT-qPCR SVV-001 biomarker panel combined with the Nanostring IO360 panel prior to enrollment in the trial. In the event of lack of availability of archival tissue, a fresh biopsy will be conducted to screen for SVV-001 biomarker positivity (Day 28 to Day −1). Pre- and post-treatment biopsies will be obtained from patients in Cohorts 6 only, and evaluated for gene expression changes using the nCounter® platform. All tumor biopsy specimens should be immediately fixed in 10% neutral buffered formalin and sent to the qualified laboratory for testing.

### Pretreatment Biopsy Samples

Biopsies will be obtained from all patients prior to treatment and will be evaluated using the RT-qPCR platform, RNAseq, and/or the nCounter® platform for a favorable SVV-001 biomarker profile to determine patient eligibility. Pretreatment tumor samples will also be evaluated using next generation DNA sequencing (eg, FoundationOne CDx or similar platform) to define tumor mutational burden (TMB), identify specific mutations in tumor suppressors or oncogenes and mutations in microsatellite instability (MSI) and deficiencies in mismatch repair genes (dMMR; MSI/dMMR is measured to give an indication of genomic instability). Both TMB and MSI/dMMR correlate with clinical efficacy of checkpoint inhibitor blockers. Pretreatment lesions will be assessed for immune infiltrating cells by IHC, expression of angiogenic and checkpoint inhibitors, type 1 IFN pathway genes using the Nanostring IO360 platform and compared with the post-treatment biopsy.

### Post-Treatment Biopsy Samples (Cohorts 6 Only)

Analyses will be performed to assess changes in the immune environment in biopsy samplesfrom injected and non-injected tumors. Possible assessments include the following:
Immunohistochemistry to assess infiltration of immune cells, eg, T cells, NaturalKiller cells, macrophages, by staining with antibodies against CD3, CD8, CD4, FoxP3, CD56, B cell (B220), macrophage (CD163), MDSC, PD-1, CXCR3
PD-1/PD-L1 and other immune checkpoint markers, CD31, Ki-67 expression on tumor and immune cells
Type I IFN pathway activation and angiogenic markers
Gene expression changes using Nanostring IO360 (or similar) to quantify tumor immune signature and other biological signatures, T-cell receptor oligoclonality
SVV-001 viral infection, replication, and impact on tumor, stroma, and endothelial cells and correlation with baseline TEM8 expression

### Plasma Analytics

Plasma specimens will be collected for cytokine analysis per the schedule in Table 30. Evaluations may include (but are not limited to): IL-2, IL-4, IL-6, IL-10, IFN-γ, TNF-α, GM-CSF, CXCL8 (IL-8), CXCL9 (Mig-1), CXCL10 (IP-10), CXCL11 (I-Tac), CCL3 (MIP-1a).

Plasma samples will also be used to quantitate circulating SVV-001 virus levels and generation of total and neutralizing antibodies against SVV-001.

### Circulating Immune Cells

At the indicated times (Table 30), blood samples will be collected for immunophenotyping of peripheral blood mononuclear cells by flow cytometry. Markers to be evaluated include: T-cell receptor V-beta changes, immune cell markers such as CD3, CD8, CD4, FoxP3, CD56, B 220 and T cell activation markers: PD-1, TIM-3, 4-1-BB, Ki-67.

### Adverse Events

### Definitions

### Adverse Event

Per the International Conference on Harmonization (ICH) E2A (Clinical Safety Data Management: Definitions and Standards for Expedited Reporting E2A), and 21 CFR 312.32 IND Safety Reporting, an AE is any untoward medical occurrence in a subject or clinical investigation subject administered a pharmaceutical product and which does not necessarily have a causal relationship with this treatment.

### Serious Adverse Event

A serious adverse event (SAE) is any untoward medical occurrence that fulfils any of the following criteria:

TABLE 28
Criteria for a serious adverse event (SAE)

| | |
|---|---|
| Fatal: | Results in death. |
| Life threatening: | Is life threatening. |
| | Note: The term “life-threatening” in the definition of “serious” refers to |
| | an event in which the subject was at risk of death at the time of the |
| | event; it does not refer to an event which hypothetically might have |
| | caused death if it were more severe. |
| Hospitalization: | Requires in-patient hospitalization or results in prolongation of an |
| | existing hospitalization. (Hospitalizations for elective medical or |
| | surgical procedures or treatments planned before the signing of |
| | informed consent in the trial or routine check-ups are not SAEs by this |
| | criterion. Admission to a palliative unit or hospice care facility is not |
| | considered to be a hospitalization. Hospitalizations or prolonged |
| | hospitalizations for scheduled therapy of the underlying cancer or trial |
| | target disease need not be captured as SAEs). |
| Disabling/ | Results in a persistent or significant disability/ incapacity or substantial |
| incapacitating: | disruption of the subject's ability to conduct normal life functions. |
| Congenital | Is a congenital anomaly or birth defect in a child or fetus of a |
| anomaly or birth | patient/subject who has been exposed to the molecule or trial |
| defect: | treatment regimen before conception or during pregnancy. |
| Medically | The AE does not meet any of the above criteria but could have |
| significant: | jeopardized the subject and might have required medical or surgical |
| | intervention to prevent one of the outcomes listed above or involves |
| | suspected transmission via a medicinal product of an infectious agent. |

### Adverse Event Severity

AE severity should be graded using the NCI-CTCAE, version 5.0.

Severity and seriousness of AEs are assessed independently. ‘Severity’ characterizes the intensity of an AE. ‘Serious’ is a regulatory definition and serves as a guide to the sponsor for defining appropriate regulatory reporting obligations (see definition for SAEs).

### Relationship of the Adverse Event to Trial Treatment

The relationship of each AE to IMP should be evaluated using the following criteria:

TABLE 29
Relationship of each AE to IMP (SVV-
001 or Nivolumab) evaluation.

| | |
|---|---|
| Related: | There is evidence to suggest a causal |
| | relationship between the drug |
| | and the AE, such as the following: |
| | An event that is uncommon and known to be |
| | strongly associated with drug exposure |
| | An event that is not commonly associated |
| | with drug exposure, but is otherwise |
| | uncommon in the population exposed |
| | to the drug |
| Unrelated: | Another cause of the AE is more |
| | plausible (eg, due to underlying |
| | disease or occurs commonly in |
| | the trial population), or a temporal |
| | sequence cannot be established |
| | with the onset of the AE and |
| | administration of the trial treatment, |
| | or a causal relationship is |
| | considered biologically implausible |

### Immunogenicity

Samples for the evaluation of SVV-001 immunogenicity (ie, total and neutralizing antibodies) will be collected according to Table 30. A qualified immunoassay conducted by a central laboratory will be used to assess neutralizing and total antibodies against SVV-001.

### Appropriateness of Measurements

The safety measures that will be used in this trial are considered standard procedures for evaluating the potential adverse effects of trial medications. Adverse events and, when applicable, clinical laboratory data will be graded using NCI CTCAE, version 5.0.

Response will be assessed according to iRECIST, which includes standard criteria for evaluating immune-related response in solid tumors. The intervals of evaluation in this protocol are appropriate for disease management.

### Analysis Populations

The population to be analyzed will include all enrolled patients who receive at least 1 dose of SVV-001. This population will be used for all analysis on the safety and clinical activity profile.

### Clinical Activity Analysis

Percent change from baseline in sum of diameters for injected and non-injected lesions will be evaluated according to iRECIST and RECIST 1.1.

Progression-free survival is defined as the time from first dose of IMP until radiographically determined disease progression or death due to any cause, whichever occurs first. Patients who are still alive or who have no progressive disease reported at the 3-year time point will be censored at their last evaluable tumor assessment.

Overall survival is defined as the time from first dose of trial medication to date of death due to any cause.

Percent change from baseline in sum of diameters for injected and non-injected lesions will be evaluated according to iRECIST.

Overall Response Rate (ORR) is defined as the percentage of patients with confirmed PR or CR based on tumor assessment as determined by iRECIST and RECIST 1.1. Patients who discontinue due to toxicity or clinical progression prior to post baseline tumor assessments will be considered as nonresponders.

Clinical benefit rate is defined as the percentage of patients with confirmed CR, PR, or stable disease. Patients who discontinue prior to post baseline tumor assessments will be considered as nonresponders.

Duration of response is defined as time from date of reported confirmed PR or CR to the date of progression and will be summarized descriptively using summary statistics. Additionally, a listing of duration of response for those patients experiencing response will be provided.

Kaplan Meier estimates and 95% confidence intervals will be presented for time to event endpoints such as PFS if sufficient numbers of events to calculate meaningful statistics are observed.

### Pharmacodynamic and Biomarker Analyses

Descriptive statistics of the mean, median, absolute number, and percent change from baseline may be evaluated for immunohistochemistry, blood tumor markers, and soluble biomarkers. Exploratory analyses may be performed to evaluate a possible correlation between each of these endpoints and disease response and/or toxicities. Additional analyses may also be performed.

### Safety Analyses

Adverse events will be coded according to Medical Dictionary for Regulatory Activities (MedDRA) version 21.0 or higher and assessed for severity using NCI-CTCAE version 5.0. Adverse events including immune-related adverse events will be summarized by system organ class and preferred term and presented in decreasing order of incidence. Dose-limiting toxicities will also be summarized by dose and cohort. The incidence of TEAEs (events with onset dates on or after the start of the IMP) will be included in incidence tables. Events with missing onset dates will be included as treatment emergent. If a patient experiences more than 1 occurrence of the same AE, the occurrence with the greatest severity and the closest association with the IMP will be used in the summary tables. Serious adverse events and AEs causing discontinuation will be tabulated. All AEs will be listed by patient, along with information regarding onset, duration, relationship and severity to IMP, action taken with IMP, treatment of event, and outcome.

Vital sign measurements, ECG data, clinical laboratory assessments, and ECOG performance status at baseline and during the trial and any changes from baseline will be examined. Treatment—emergent changes in key laboratory parameters will be identified. Clinical laboratory data will be summarized for each time point that specimens are collected. Changes from baseline for clinical laboratory values may also be explored.

Summary tables will be provided for concomitant medications initiated prior to trial enrollment or during the trial period.

NEC Cohort: An interim analysis will be performed when 46 events (progression or death) have been observed. If the observed log-rank test p-value is less than 0.003, the trial will stop for efficacy. If the observed log-rank test p-value is greater than 0.149, the trial is advised to stop for futility (non-binding). The p-value threshold for the log-rank test used in the final analysis of PFS is 0.049.

NET Cohort: An interim analysis will be performed when 100 events (progression or death) have been observed. If the observed log-rank test p-value is less than 0.005, the trial will stop for efficacy. If the observed log-rank test p-value is greater than 0.162, the trial is advised to stop for futility (non-binding). The p-value threshold for the log-rank test used in the final analysis of PFS is 0.051.

TABLE 30
Schedule of Assessments - Single and Multiple Dose Cohorts in Part I (Part IA and Part IB)

| | | | Treatment | | | | | | | | | |
|---|---|---|---|---|---|---|---|---|---|---|---|---|
| | | | Period | | | | | | | | | |
| | | | | | | | | | | Q4 weekly | | |
| | | | SVV-001 Treatment (12 weeks) + | | | | | | | visits | | |
| | Screening | | ICB (Nivo X4 doses followed by | | | | | | | for IV | | |
| Trial Assessment | Visit | | q Monthly Nivo) | | | | | | | Nivolumab | EoT/ | |
| Day | — | 1 | 8 | 15 | 29 | 36 | 43 | 57 | 71 | Infusion^{m} | ET | F/U |
| Injection Number | | 1 | — | 2 | 3 | | 4 | 5 | 6 | | | |
| Window (days) | −28 to −1 | −1 | ±1 | ±1 | ±1 | | ±3 | ±3 | ±3 | ±3 | ±7 | |
| Informed consent^{a} | X | | | | | | | | | | | |
| Demographics | X | | | | | | | | | | | |
| Eligibility screening | X | X | | | | | | | | | | |
| Medical history^{b} | X | X | | | | | | | | | | |
| Physical examination^{c} | X | | | | | | | | | | X | |
| Height, weight^{d} | X^{d} | X | | | | | | | | X | X | |
| Vital sign measurements^{e} | X | X | X | X | X | X | X | X | X | X | X | |
| ECOG^{f} | X | X | | | | X | | | X | X | X | |
| 12-lead ECG^{g} | X | X | | | X | | | | X | | X | |
| Pregnancy testing^{h} | X | | | | | | | | | | X | |
| Tumor biopsyⁱ | X | | | X | X | | | | | | | |
| Hematology and biochemistry^{j} | X | X | | X | X | X | X | X | X | X | X | |
| (CBC/CMP +/− 1 hour) | | | | | | | | | | | | |
| Coagulation and urinalysis^{k} | X | | | | | | | | | | X | |
| SVV-001 administration^{l} (+/− 2 | | X | | X | X | | X | X | X | | | |
| hours) | | | | | | | | | | | | |
| Nivolumab administration^{m} (+/− | | | | X | X | | X | X | | X | | |
| 2 hours) | | | | | | | | | | | | |
| Peripheral blood sampleⁿ | X | X | X | X | X | X | | X | X | | X | |
| Immunogenicityⁿ | | X | X | X | X | | | X | X | | X | X |
| Cytokine sampleⁿ | | X | X | X | X | | X | X | X | | X | |
| Viral burden sampleⁿ (sample | | Week 1 (T, W, Th, F) | | | | | | | | | X | |
| collection +/− 30 minutes; | | Week 2-3 (M, W, F) | | | | | | | | | | |
| | | Week 4-6 (M, Th) | | | | | | | | | | |
| | | Week 7 and beyond (M) | | | | | | | | | | |
| Viral sheddingº (sample | | Sampling on day 1, 3, 7, 10 and then weekly | | | | | | | | | | |
| collection +/− 1 day) | X | | | | | | | | X | Q8W for the | X | |
| Radiology evaluations (CT | | | | | | | | | | first 6 months, | | |
| or MRI)^{p} (+/− 7 days) | | | | | | | | | | then Q12W | | |
| | | | | | | | | | | thereafter | | |

| | |
|---|---|
| Concomitant medications | ← . . . → |
| Adverse events | ← . . . → |

Abbreviations: D1 = Day 1; CT = computed tomography; ECG = electrocardiogram; ECOG = Eastern Cooperative Oncology Group; EoT = End of Trial; ET =Early Termination; F/U = follow-up; ICB = immune checkpoint blocker; IHC = immunohistochemistry; IMP = investigational medicinal product; MRI = magnetic resonance imaging; NIVO = nivolumab; Q1W = every week; Q2W = every 2 weeks; Q4W = every 4 weeks; Q6W = every 6 weeks; Q12W = every 12 weeks; TEM8 = tumor endothelial marker 8.
^{a}Informed consent to be obtained before any other trial procedures are performed.
^{b}Medical history to include previous cancer therapies, cancer history, surgeries, and past and ongoing concomitant illnesses.
^{c}A complete physical examination is required at screening and end of trial/early termination. Directed physical examinations, which are limited to problem focused review of symptoms and major organ systems, may be completed at any time during the trial at the investigator's discretion.
^{d}Height to be obtained at screening only.
^{e}Vital sign measurements include temperature, blood pressure, and pulsc (at supine position). Temperature measurement will be obtained as clinically indicated.
^{f}ECOG performance status: at screening, Day 1 of each cycle, on Day 71 of SVV-001 cycle, and end of trial/early termination
^{g}12-lead ECG should be performed in triplication after the patient has been in the supine position for at least 10 minutes.
^{h}Pregnancy testing should be performed in women of childbearing potential only. A serum pregnancy test is required during screening. A urine or serum pregnancy test is acceptable at the end of trial or early termination. Any positive urine test should be confirmed with a serum pregnancy test.
ⁱPretreatment SVV-001 biomarker screening tumor biopsy samples should use archival sample (preferred), or if not available, biopsy is to be obtained prior to enrollment from Day −1 to Day −28 and screened for expression of the SVV-001 biomarker profile using the nanostring nCounter ® platform. Pretreatment biopsies are obtained from all patients on Day −1 to Day −28 and post-treatment biopsies are obtained from Cohort 6 patients on Day 15 or Day 29.
^{j}Hematology and serum chemistry . Blood draw is prior to IMP (SVV-001) administration.
^{k}Coagulation and urinalysis.
^{l}SVV-001 is injected intratumorally once on Day 1 in Part IA (Cohort 1-3) and every 2 weeks for 12 weeks in Part IB (Cohort 4-6)
^{m}The nivolumab dose will be 240 mg administered intravenously (IV) every 2 weeks during SVV treatment. Following that, patients will continue on 480 mgnivolumab IV every 4 weeks until progression of disease, end of treatment, or unacceptable toxicity. For Part IA and Part IB, nivolumab will start on Day 15.
ⁿPeripheral blood draw samples are obtained for leukocyte cellular analysis by flow cytometry and plasma samples are used for analysis of immunogenicity (anti-SVV-001 antibodies), cytokine sampling, and virus quantification assay (Enough sample will be collected to test both virus quantification assay and Endpoint Dilution Assay at each timepoint. Endpoint Dilution Assay will be assessed only if positive).
^{o}Virus shedding will be assessed by sampling stool (using a fecal swab) utilizing a PCR assay taken at indicated time points. Shedding will be evaluated in patients treated with SVV-001 (Phase 1 only). If positive, Endpoint Dilution Assay in the fecal sample will be done, and patients' stool will be collected every week (+/− 1 day) after the last dose until it's negative.
^{p}Radiology (CT or MRI scan) evaluations of tumor response: Performed during screening and during the trial every 8 weeks for the first 6 months, and then every 12 weeks until discontinuation of trial treatment, disease progression, death, or initiation of further systemic cancer therapy, including radiation therapy, whichever occurs earlier. CT or MRI scan will be performed within 1 week prior to the start of the next cycle. Unscheduled scan can be performed anytime, if needed to confirm disease progression. Radiology assessment for early termination/end of trial, should ONLY be performed if ≥ 12 weeks have passed from the previous scheduled CT/MRI scan. Same assessment method and same technique should be used on each patient while on trial.

TABLE 31
ECOG Performance Status Scale.
ECOG

| Score | Description |
|---|---|
| 0 | Normal activity. Fully active, able to carry on |
| | all pre-disease performance without restriction. |
| 1 | Symptoms, but ambulatory. Restricted in |
| | physically strenuous activity, but ambulatory and able |
| | tocarry out work of a light or sedentary nature |
| | (eg, light housework, office work). |
| 2 | In bed <50% of the time. Ambulatory and capable |
| | of all self-care, but unable to carry |
| | out anywork activities. |
| | Up and about more than 50% of waking hours. |
| 3 | In bed >50% of the time. Capable of only |
| | limited self-care, confined to bed or chair |
| | more than50% of waking hours. |
| 4 | 100% bedridden. Completely disabled. |
| | Cannot carry on any self-care. Totally |
| | confined to bed or chair. |
| 5 | Dead. |

### Evaluation of Tumor Response

This includes selected sections from the Immune-Related Response Evaluation Criteria in Solid Tumors (irRECIST).

### Measurability of Tumor at Baseline

### Definitions

At baseline, tumor lesions/lymph nodes will be categorized as measurable or non-measurable as described below.

### Injectable Lesions

Patients will be enrolled with at least one lesion that is at least 10 mm in longest diameter and deemed suitable and safe for intratumoral injection. Care will be taken to avoid lesions invading a major neurovascular structure (eg, carotid artery) or other key anatomical structure (eg, pulmonary airway) and to minimize the potential adverse effects of post-treatment tumor swelling or necrosis on these structures. Injections will be administered under imaging guidance, either computed tomography (CT) or ultrasound.

### Measurable Tumor Lesions

Tumor Lesions. Tumor lesions must be accurately measured in at least one dimension (longest diameter in the plane of measurement is to be recorded) with a minimum size as follows:
10 mm by CT or magnetic resonance imaging (MRI) scan (CT/MRI scan slice thickness/interval no greater than 5 mm)
10 mm calliper measurement by clinical examination (lesions that cannot be accurately measured with calipers should be recorded as non-measurable)
20 mm by chest X-ray
Malignant Lymph Nodes. To be considered pathologically enlarged and measurable, a lymph node must be ≥15 mm in the short axis when assessed by CT scan (CT scan slice thickness recommended to be no greater than 5 mm). At baseline and follow-up, only the short axis will be measured and followed.

### Non-Measurable Tumor Lesions

Non-measurable tumor lesions encompass small lesions (longest diameter <10 mm or pathological lymph nodes with short axis ≥10 but <15 mm), as well as truly non-measurable lesions. Lesions considered truly non-measurable include leptomeningeal disease, ascites, pleural or pericardial effusion, inflammatory breast disease, lymphangitic involvement of skin or lung, and abdominal mass/abdominal organomegaly identified by physical examination that is not measurable by reproducible imaging techniques.

### Special Considerations Regarding Lesion Measurability

Bone lesions, cystic lesions, and lesions previously treated with local therapy require particular comment, as outlined below.

### Bone Lesions:

Bone scan, positron emission tomography (PET) scan, or plain films are not considered adequate imaging techniques for measuring bone lesions. However, these techniques can be used to confirm the presence or disappearance of bone lesions.
Lytic bone lesions or mixed lytic-blastic lesions, with identifiable soft tissue component
   ≥10 mm can be selected as target lesions.
Blastic bone lesions are non-measurable.

### Brain Lesions:

Brain lesions detected on brain scans can be considered as both target or non-target lesions.

### Cystic and Necrotic Lesions:

Lesions that are partially cystic or necrotic can be selected as target lesions. The longest diameter of such a lesion will be added to the Total Measured Tumor Burden (TMTB) of all target lesions at baseline. If other lesions with a non-liquid/non-necrotic component are present, those should be preferred.

Lesions with Prior Local Treatment:

During target lesion selection the radiologist will consider information on the anatomical sites of previous intervention (eg, previous irradiation, radiofrequency-ablation, transarterial chemoembolization, surgery). Lesions undergoing prior intervention will not be selected as target lesions unless there has been a demonstration of progress in the lesion.

### No Disease at Baseline:

If a patient has no measurable and no non-measurable disease at baseline the radiologist will assign‘No Disease’ (irND) as the overall tumor assessment for any available follow-up time points unless new measurable lesions are identified and contribute to the TMTB.

### Guidelines for Evaluation of Measurable Lesions

### Measurement of Lesions

All measurements should be recorded in metric notation, using calipers if clinically assessed. All baseline evaluations should be performed as close as possible to the treatment start and never more than 4 weeks before the beginning of the treatment.

### Method of Assessment

The same method of assessment and the same technique should be used to characterize each identified and reported lesion at baseline and during the trial. Imaging-based evaluation should always be the preferred option.

Clinical Lesions. Clinical lesions will only be considered measurable when they are superficial and ≥10 mm in diameter as assessed using calipers (eg, skin nodules). For the case of skin lesions, documentation by color photography including a ruler to estimate the size of the lesion is suggested.

Chest X-Ray. Chest CT is preferred over chest X-ray, particularly when progression is an important endpoint, since CT is more sensitive than X-ray, particularly in identifying new lesions. However, lesions on a chest X-ray may be considered measurable if they are clearly defined and surrounded by aerated lung.

CT, MRI. CT is the best currently available and reproducible method to measure lesions selected for response assessment. This guideline has defined measurability of lesions on CT scan based on the assumption that CT slice thickness is 5 mm or less. When CT scans have slice thicknesses greater than 5 mm, the minimum size for a measurable lesion should be twice the slice thickness. MRI is also acceptable. If prior to enrollment it is known that a subject is unable to undergo CT scans with intravenous (IV) contrast because of allergy or renal insufficiency, the decision as to whether a non-contrast CT or MRI (without IV contrast) will be used to evaluate the subject at baseline and during the trial should be guided by the tumor type under investigation and the anatomic location of the disease. For subjects who develop contraindications to contrast after baseline contrast CT is done, the decision as to whether non-contrast CT or MRI (enhanced or non-enhanced) will be performed should also be based on the tumor type and the anatomic location of the disease and should be optimized to allow for comparison with the prior studies if possible. Each case should be discussed with the radiologist to determine if substitution of these other approaches is possible and, if not, the subject should be considered not evaluable from that point forward. Care must be taken in measurement of target lesions on a different modality and interpretation of non-target disease or new lesions since the same lesion may appear to have a different size using a new modality.

Ultrasound. If new lesions are identified by ultrasound in the course of the trial, confirmation by CT or MRI is advised. If there is concern about radiation exposure at CT, MRI may be used instead of CT in selected instances.

Endoscopy, Laparoscopy can be useful to confirm complete pathological response when biopsies are obtained or to determine relapse in trials where recurrence following complete response or surgical resection is an endpoint.

Tumor Markers. Tumor markers alone cannot be used to assess objective tumor response. If markers are initially above the upper normal limit, however, they must normalize for a patient to be considered in complete response.

Cytology, Histology. These techniques can be used to differentiate between immune related (ir)PR and (ir)CR in rare cases if required by protocol (for example, residual lesions in tumor types such as germ cell tumors, where known residual benign tumors can remain). When effusions are known to be a potential adverse effect of treatment (eg, with certain taxane compounds or angiogenesis inhibitors), the cytological confirmation of the neoplastic origin of any effusion that appears or worsens during treatment can be considered if the measurable tumor has met criteria for response or stable disease in order to differentiate between response (or stable disease) and progressive disease.

### Tumor Response Evaluation

### Assessment of Overall Tumor Burden and Measurable Disease

To assess objective response or future progression, it is necessary to estimate the overall tumor burden at baseline and use this as a comparator for subsequent measurements. Measurable disease is defined by the presence of at least one measurable lesion, as detailed above.

### Baseline Documentation of Target Lesions

All target lesions selected for RECIST1.1 or irRECIST response determination should be treated with SVV-001 by ultra-sound, CT or endoscopic guided intra-tumoral injection.

For the purpose of SVV-001 and nivolumab combination treatment effect evaluation, only the treated target lesion(s) will be considered to evaluate whether or not a patient obtained an objective response to treatment. Patients will be considered as having obtained such a response if they exhibit a CR or PR as an overall response restricted to the treated target tumor sites at any time during the trial (during treatment or during the 12-month follow-up period). It must also be determined if this response can be attributable to the SVV-001 treatment and not a result of any adjuvant treatment received during that period that is not allowed by the protocol (eg, surgery, chemotherapy, radiation therapy, etc.).

Target lesions as defined by RECIST 1.1 are no more than 5 but at least 2 target lesions per organ. RECIST 1.1 criteria for tumor response for measurable and non-measurable disease will be applied as listed below.
a) Measurable Disease using shortest diameter (for lymph nodes) or longest diameter (tumor lesions) of target lesions. Target lesions as defined by RECIST 1.1 are no more than 5 but at least 2 lesions.

CR: The disappearance of all known disease determined by two observations not less than four weeks apart.

PR: 30% or more decrease in the total sum of all measurements as defined above as determined by two observations not less than four weeks apart.

Stable Disease: A less than 30% decrease or a less than 20% increase in the total sum of all measurements as defined above.

Progressive disease: A 20% or more increase in the total measurements of all lesions as defined above.
b) Evaluable, Non-measurable Disease

CR: Complete disappearance of all known disease for at least four weeks. PR: Estimated decrease in tumor size of 30% or more for at least four weeks.

No change: No significant change for at least four weeks. This includes stable disease, estimated decrease of less than 50%, and lesions with estimated increase of less than 25%.

Progressive disease: An estimated increase of 20% or more.

### Baseline Documentation of “Target” and “Non-Target” Lesions

All measurable lesions up to a maximum of two lesions per organ and 10 lesions in total, representative of all involved organs should be identified as target lesions and recorded and measured at baseline.

Target lesions should be selected on the basis of their size (lesions with the longest diameter) and their suitability for accurate repeated measurements (either by imaging techniques or clinically).

A sum of the longest diameter (LD) for all target lesions will be calculated and reported as the baseline sum LD. The baseline sum LD will be used as reference by which to characterize the objective tumor.

All other lesions (or sites of disease) should be identified as non-target lesions and should also be recorded at baseline. Measurements of these lesions are not required, but the presence or absence of each should be noted throughout follow-up.

At the baseline tumor assessment, the sum of the products of the two largest perpendicular diameters (SPD) of all index lesions (5 lesions per organ, up to 10 visceral lesions and 5 cutaneous index lesions) is calculated. At each subsequent tumor assessment, the SPD of the index lesions and of new, measurable lesions (≥5×5 mm; up to 5 new lesions per organ: 5 new cutaneous lesions and 10 visceral lesions) are added together to provide the total tumor burden:

Tumor Burden=SPD_{index lesions}+SPD_{new, measurable lesions}

A comparison of the use of SPD in WHO criteria versus the use of tumor burden in immune-related response criteria (irRC) is presented in Table 30.

TABLE 32
Comparison Between WHO Criteria and Immune-related Response Criteria

| | WHO | irRC |
|---|---|---|
| New, measurable | Always represent PD | Incorporated into tumor burden |
| lesions (ie, ≥5 × 5 mm) | | |
| New, non- | Always represent PD | Do not define progression (but |
| measurable lesions | | preclude irCR) |
| (ie, <5 × 5 mm) | | |
| Non-index | Changes contribute to defining BOR | Contribute to defining irCR |
| lesions | of CR, PR, SD, and PD | (complete disappearance |
| | | required) |
| CR | Disappearance of all lesions in 2 | Disappearance of all lesions in 2 |
| | consecutive observations not less than | consecutive observations not less |
| | 4 weeks apart | than 4 weeks apart |
| PR | ≥50% decrease in SPD of all index | ≥50% decrease in tumor burden |
| | lesions compared with baseline in 2 | compared with baseline in 2 |
| | observations at least 4 weeks apart, | observations at least 4 weeks apart |
| | in absence of new lesions or | |
| | unequivocal progression of non- | |
| | index lesions | |
| SD | 50% decrease in SPD compared with | 50% decrease in tumor burden |
| | baseline cannot be established nor | compared with baseline cannot be |
| | 25% increase compared with nadir, in | established nor 25% increase |
| | absence of new lesions or unequivocal | compared with nadir |
| | progression of non-index lesions | |
| PD | At least 25% increase in SPD | At least 25% increase in tumor |
| | compared with nadir and/or | burden compared with nadir (at any |
| | unequivocal progression of non-index | single timepoint) in 2 consecutive |
| | lesions and/or appearance of new | observations at least 4 weeks apart |
| | lesions (at any single time point) | |

BOR = best overall response;
CR = complete response;
irCR = immune-related complete response;
irRC = immune-related response criteria;
PD = progressive disease;
SD = stable disease;
SPD = sum of the products of the two largest perpendicular diameters;
WHO = World Health Organization

### Baseline Documentation of Non-Target Lesions

All lesions or sites of disease not recorded as target lesions should be recorded as non-target lesion sat baseline. There is no limit to the number of non-target lesions that can be recorded at baseline.

### Follow-Up Recording of Target and New Measurable Lesion Measurements

The longest diameters of non-nodal target and new non-nodal measurable lesions, and short axes of nodal target and new nodal measurable lesions will be recorded. Together they determine the TMTB at follow-up.

### Definition of Measurable New Lesions

In order to be selected as new measurable lesions (≤2 lesions per organ, ≤5 lesions total, per timepoint), new lesions must meet criteria as defined for baseline target lesion selection and meet the same minimum size requirements of 10 mm in long diameter and minimum 15 mm in short axis for new measurable lymph nodes. New measurable lesions shall be prioritized according to size, and the largest lesions shall be selected as new measured lesions.

### Non-Target Lesion Assessment

The response of non-target lesions primarily contributes to the overall response assessments of immune-related complete response (irCR) and irNon-CR/Non-progressive disease (irNN). Non-target lesions do not affect immune-related partial response (irPR) and immune-related stable disease (irSD) assessments. Only a massive and unequivocal worsening of non-target lesions alone, even without progress in the TMTB is indicative of immune-related progressive disease (irPD).

### New Non-Measurable Lesions Definition and Assessment

All new lesions not selected as new measurable lesions are considered new non-measurable lesions and are followed qualitatively. Only a massive and unequivocal progression of new non-measurable lesions leads to an overall assessment of irPD for the timepoint. Persisting new non-measurable lesions prevent irCR.

### Immune-Related Response Criteria

### Immune-Related Response Criteria Tumor Assessments

This section provides the definitions of the criteria used to determine objective tumor response for target lesions.
Immune-related complete response (irCR)—complete disappearance of all lesions (whether measurable or not, and no new lesions) confirmation by a repeat, consecutive assessment no less than 4 weeks from the date first documented.
Immune-related partial response (irPR)—decrease in tumor burden ≥50% relative to baseline confirmed by a consecutive assessment at least 4 weeks after first documentation.
Immune-related stable disease (irSD)—failure to meet criteria for irCR or irPR in the absence of irPD.
irNon-CR/Non-PD (irNN)—no target disease was identified at baseline and at follow-up the patient fails to meet criteria for irCR or irPD.
Immune-related progressive disease (irPD)—increase in tumor burden ≥25% relative tonadir (minimum recorded tumor burden) confirmation by a repeat, consecutive assessment no less than 4 weeks from the date first documented.
Immune-related non-evaluable (irNE)—used in exceptional cases where insufficient data exists.
Immune-related no disease (irND)—in adjuvant setting when no disease is detected.

### Summary and Additional Guidance

### Total Measured Tumor Burden

Baseline-selected target lesions and new measurable lesions should NOT be assessed separately. Measurements of those lesions should be combined into the Total Measured Tumor Burden (TMTB), and one combined assessment provided.

### New Measurable Lesions

According to irRC a measurable new lesion has to be at least 5 mm×5 mm to be selected as an index lesion. For bidimensional measurements this threshold was acceptable. In irRECIST, criteria for unidimensional lesion measurement apply to both target and new measurable lesions: a minimum 10 mm in the longest diameter for non-nodal lesions, and a minimum 15 mm in short axis for lymph nodes. Smaller lesions contribute to the non-target or new nonmeasurable tumor burden, but do not get measured.

irPR if no Target Lesions;

If new measurable lesions appear in patients with no target lesions at baseline, irPD will be assessed. That irPD timepoint will be considered a new baseline, and all subsequent timepoints will be compared to it for response assessment. irPR is possible if the TMTB of new measurable lesions decreases by ≥30% compared to the first irPD documentation.

irPR in Adjuvant Studies

irRECIST can be used in the adjuvant setting, in patients with no visible disease on CT/MRI scans. The appearance of new measurable lesion(s) automatically leads to an increase in TMTB by 100% and leads to irPD. These patients can achieve a response if the TMTB decreases at follow-up, as a sign of delayed response.

### Non-Target Lesions

In alignment with RECIST 1.1, baseline selected non-target lesions can never convert to measurable lesions, not even if they increase in size at subsequent timepoints and become measurable. Only true new lesions can be measured and contribute to the TMTB.

### Exemplary Approach

A patient has multiple lung metastases, all smaller than 10 mm and selected as non-target lesions at baseline. If, at a subsequent timepoint some of these non-target lesions increase and become >10 mm, and one new lesion >10 mm appears, only the new measurable lesion will contribute to the TMTB, and not the baseline selected non target lesion that increased in size. Otherwise such an increase would make persisting non-target lesions switch into the new measurable lesion category which would be inaccurate, as the lesion existed at baseline.

irPD Based on Non-Target Lesions

Unlike irRC that neglect non-target lesions for the assessment of irPD, in irRECIST a substantial and unequivocal increase of non-target lesions is indicative of progression.

irPD Based on New Non-Measurable Lesions

According to irRC, a patient with multiple new lesions of 9 mm would be considered non-PD, whereas a patient with just one new lesion of 10 mm may be assessed as irPD if the TMTB of such a patient increases ≥25% compared to nadir. According to irRECIST, the reviewer may assign irPD for the patient with multiple new lesions of 9 mm if they are considered to be a sign of unequivocal, massive worsening (see section on Follow-up: New Non-Measurable Lesions Definition and Assessment).

irPD Confirmation

Progression confirmation no less than 4 weeks after the initial irPD assessment is recommended especially in case of marginal disease growth and if the first irPD assessment is within the compound-specific tumor flare window.

### Evaluation of Best Overall Response

### Time Point Response

Percentage changes in tumor burden per assessment time point describe the size and growth kinetics of both conventional and new, measurable lesions as they appear. At each tumor assessment, the response in index and new, measurable lesions is defined based on the change in tumor burden (after ruling out irPD). Decreases in tumor burden must be assessed relative to baseline measurements (ie, the SPD of all index lesions at screening). The irRC were derived from WHO criteria and, therefore, the thresholds of response remain the same (Table 31). However, their RC response categories have been modified from those of WHO criteria as detailed in Table 30 and Table 31.

TABLE 33
Derivation of Immune-Related Response
Criteria Overall Responses.

| Measurable | | | |
|---|---|---|---|
| Response | Non-measurable Response | | |
| Index and new, | | New, Non- | |
| measurable lesions | Non-index | measurable | Overall Response |
| (tumor burden),^{a} % | Lesions | Lesions | Using irRC |
| ↓100 | Absent | Absent | irCR^{b} |
| ↓100 | Stable | Any | irPR^{b} |
| ↓100 | Unequivocal | Any | irPR^{b} |
| | progression | | |
| ↓≥50 | Absent/stable | Any | irPR^{b} |
| ↓≥50 | Uncquivocal | Any | irPR^{b} |
| | progression | | |
| ↓<50 to <25↑ | Absent/stable | Any | irSD |
| ↓50 to <25↑ | Unequivocal | Any | irSD |
| | progression | | |
| ≥25? | Any | Any | irPD^{b} |

irCR = immune-related complete response;
irPR = immune-related partial response,
irSD = immune-related stable disease
^{a}Decreases assessed relative to baseline, including measurable lesions only (>5 × 5 mm).
^{b}Assuming responsc (irCR) and progression (irPD) arc confirmed by a second, consecutive assessment at least 4 weeks apart.

### Missing Assessments and Not-Evaluable Designation

When no imaging/measurement is done at all at a particular time point, the subject is not evaluable at that time point. If only a subset of lesion measurements are made at an assessment, usually the case is also considered not evaluable at that time point unless a convincing argument can be made that the contribution of the individual missing lesion(s) would not change the assigned time point response.

If one or more target lesions were not assessed either because the scan was not done or the scan could not be assessed because of poor image quality or obstructed view, the response for target lesions should be “unable to assess” since the subject is not evaluable. Similarly, if one or more non-target lesions are not assessed, the response for non-target lesions should be “unable to assess” except where there is clear progression. Overall response would be “unable to assess,” if either the target response or the non-target response is “unable to assess” except where this is clear evidence of progression, as this equates with the case being not evaluable at that time point.

### Best Overall Response: All Time Points

The best overall response is determined once all the data for the subject is known.

Best response determination in trials where confirmation of complete or partial response is not required: Best response in these trials is defined as the best response across all time points (for example, a subject who has irSD at first assessment, irPR at second assessment, and irPD on last assessment has a best overall response of irPR). When irSD is believed to be best response, it must also meet the protocol specified minimum time from baseline. If the minimum time is not met when irSD is otherwise the best time point response, the patient's best response depends on the subsequent assessments. For example, a subject who has irSD at first assessment, irPD at second and does not meet minimum duration for irSD, will have a best response of irPD. The same subject lost to follow-up after the first irSD assessment would be considered unevaluable at a subsequent time point as specified in the protocol (generally 4 weeks later). In this circumstance, the best overall response can be interpreted as in Table 34.

TABLE 34
Best Overall Response When Confirmation
of irCR and irPR required.

| Overall | Overall Response | |
|---|---|---|
| Response First | Subsequent Time | |
| Time Point | Point | Best Overall Response |
| irCR | irCR | irCR |
| irCR | irPR | irSD, irPD, or IRPR^{a} |
| irCR | irSD | irSD provided minimum criteria for |
| | | irSD duration met, otherwise, irPD |
| irCR | irPD | irSD provided minimum criteria for |
| | | irSD duration met, otherwise, irPD |
| irCR | irNE | irSD provided minimum criteria for |
| | | irSD duration met, otherwise, NE |
| irPR | irCR | irPR |
| irPR | irPR | irPR |
| irPR | irSD | irSD |
| irPR | irPD | irSD provided minimum criteria for |
| | | irSD duration met, otherwise, irPD |
| irPR | irNE | irSD provided minimum criteria for |
| | | irSD duration met, otherwise irNE |
| irNE | irNE | irNE |

irCR = immune-related complete response,
irNE = immune-related complete not evaluable;
irPD = immune-related complete progressive disease,
irPR = immune-related complete partial response;
irSD = immune-related complete stable disease
^{a}If a irCR is truly met at first time point, then any disease seen at a subsequent time point, even disease meeting irPR criteria relative to baseline, makes the disease irPD at that point (since disease must have reappeared after irCR). Best response would depend on whether minimum duration for irSD was met. However, sometimes ‘irCR’ may be claimed when subsequent scans suggest small lesions were likely still present and in fact the patient had irPR, not irCR at the first time point. Under these circumstances, the original irCR should be changed to irPR and the best response is irPR.

### Special Notes of Response Assessment

When nodal disease is included in the sum of target lesions and the nodes decrease to “normal” size (<10 mm), they may still have a measurement reported on scans. This measurement should be recorded even though the nodes are normal in order not to overstate progression should it be based on increase in size of the nodes. As noted earlier, this means that subjects with irCR may not have a total sum of “zero” on the CRF.

Subjects with a global deterioration of health status requiring discontinuation of treatment without objective evidence of disease progression at that time should be reported as “symptomatic deterioration.” Every effort should be made to document objective progression even after discontinuation of treatment. Symptomatic deterioration is not a descriptor of an objective response; it is a reason for stopping trial therapy. The objective response status of such subjects is to be determined by evaluation of target and non-target disease.

For equivocal findings of progression (eg, very small and uncertain new lesions; cystic changes or necrosis in existing lesions), treatment may continue until the next scheduled assessment. If at the next scheduled assessment, progression is confirmed, the date of progression should be the earlier date when progression was suspected.

In studies for which subjects with advanced disease are eligible (ie, primary disease still or partially present), the primary tumor should also be captured as a target or non-target lesion, as appropriate. This is to avoid an incorrect assessment of irCR if the primary tumor is still present but not evaluated as a target or non-target lesion.

### CONFIRMATORY MEASUREMENT/DURATION OF RESPONSE

### Confirmation

In non-randomized trials where response is the primary endpoint, confirmation of irPR and irCR is required to ensure responses identified are not the result of measurement error. This will also permit appropriate interpretation of results in the context of historical data where response has traditionally required confirmation in such trials. However, in all other circumstances, ie, in randomized trials (Phase 2 or 3) or studies where stable disease or progression are the primary endpoints, confirmation of response is not required since it will noted value to the interpretation of trial results. However, elimination of the requirement for response confirmation may increase the importance of central review to protect against bias, in particular in studies which are not blinded.

In the case of irSD, measurements must have met the irSD criteria at least once after trial entry at a minimum interval (in general not less than 6-8 weeks) that is defined in the trial protocol.

### Duration of Overall Response

The duration of overall response is measured from the time measurement criteria are first met for irCR/irPR (whichever is first recorded) until the first date that recurrent or progressive disease is objectively documented (taking as reference for progressive disease the smallest measurements recorded on trial).

The duration of overall complete response is measured from the time measurement criteria are first met for CR until the first date that recurrent disease is objectively documented.

### Duration of Stable Disease

Stable disease is measured from the start of the treatment (in randomized trials, from date of randomization) until the criteria for progression are met, taking as reference the smallest sum on trial (if the baseline sum is the smallest, this is the reference for calculation of irPD).

The clinical relevance of the duration of stable disease varies in different studies and diseases. If the proportion of patients achieving stable disease for a minimum period of time is an endpoint of importance in a particular trial, the protocol should specify the minimal time interval required between two measurements for determination of stable disease.

Note: The duration of response and stable disease as well as the progression-free survival are influenced by the frequency of follow-up after baseline evaluation. It is not in the scope of this guideline to define a standard follow-up frequency. The frequency should take into account many parameters including disease types and stages, treatment periodicity and standard practice. However, these limitations of the precision of the measured endpoint should be taken into account if comparisons between trials are to be made.

### ILLUSTRATIVE EMBODIMENTS

Provided here are illustrative embodiments of the disclosed technology. These embodiments are illustrative only and do not limit the scope of the present disclosure or of the claims attached.

Embodiment 1. a Method of Treating a Cancer in a Human Subject in Need Thereof Comprising:
administering to the subject an effective amount of a Seneca Valley Virus (SVV) and an immune checkpoint inhibitor during a treatment period lasting until cancer progression, end of treatment, or unacceptable toxicity,
   wherein the subject has cancer expressing one or more biomarkers or each of the biomarkers selected from one of the following biomarker profiles:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |
| 15 | ELN, CD248, COL25A1, ALPL, SEMA5B, ATP1B2, NEUROD1, |
| | VAT1L, MFAP4, |

and expressing ANTXR1.

Embodiment 2. The method of embodiment 1, wherein the cancer expresses EFS, MFAP4, NTN3, SOX11, NEFH, CNTFR, and JPH4 at at least about two-fold, at least about three-fold, at least about four-fold, at least about five-fold, at least about six-fold, at least about seven-fold, at least about eight-fold, at least about nine-fold, at least about ten-fold, at least about 11-fold, at least about 12-fold, at least about 13-fold, at least about 14-fold, or at least about 15-fold greater expression level relative to a housekeeping gene or relative to a control expression level.

Embodiment 3. The method of embodiment 1 or 2, wherein the expression of the one or more biomarkers is increased in cancer cells sensitive to treatment with SVV.

Embodiment 4 The method of embodiment 3, wherein the cancer expresses the one or more biomarkers at at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold greater expression level when compared to expression level of the of the one or more biomarkers in cancers that are resistant to treatment with SVV.

Embodiment 5. The method of any one of the preceding embodiments, wherein the cancer expresses EFS, MFAP4, NTN3, SOX11, NEFH, CNTFR, JPH4, and ANTXR1 as determined using a gene expression analysis selected from RNA sequencing (RNAseq), nCounter® platform, and quantitative polymerase chain reaction (qPCR).

Embodiment 6. The method of any one of embodiments 1-5 further comprising detecting the expression in a biological sample from the subject.

Embodiment 7. The method of any one of embodiments 1-6, wherein the biological sample is a biopsy of the cancer.

Embodiment 8. The method of embodiment 7, wherein the biological sample is a formalin-fixed paraffin-embedded (FFPE) tumor sample.

Embodiment 9 A method of treating a cancer in a human subject in need thereof comprising administering an effective amount of SVV and an immune checkpoint inhibitor when the cancer expresses ANTXR1 and one or more biomarker profiles selected from the following biomarker profiles:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |
| 15 | ELN, CD248, COL25A1, ALPL, SEMA5B, ATP1B2, NEUROD1, |
| | VAT1L, MFAP4, |

and wherein the administering is during a treatment period lasting until cancer progression, end of treatment, or unacceptable toxicity.

Embodiment 10. The method of any one of the preceding embodiments, wherein the immune checkpoint inhibitor is a programmed death receptor-1 (PD-1) inhibitor.

Embodiment 11. The method of any one of the preceding embodiments, wherein the immune checkpoint inhibitor is nivolumab.

Embodiment 12. The method of any one of embodiments 9 to 11, wherein the cancer expresses EFS, MFAP4, NTN3, SOX11, NEFH, CNTFR, and JPH4 at at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold greater expression level relative to a housekeeping gene, or relative to a control expression level.

Embodiment 13. The method of any one of embodiments 9-12, wherein the expression is increased relative to the expression of the one or more biomarker profiles in cancers that are resistant to treatment with SVV.

Embodiment 14. The method of any one of embodiments 9-13, wherein the method further comprises detecting the expression of the one or more biomarkers comprising EFS, MFAP4, NTN3, SOX11, NEFH, CNTFR, JPH4, and ANTXR1 using a gene expression analysis selected from RNA sequencing (RNAseq), nCounter® platform, and quantitative polymerase chain reaction (qPCR).

Embodiment 15. The method of any one of embodiments 1-14, wherein SVV is encoded by SEQ ID NO: 2.

Embodiment 16. The method of embodiment 15, wherein SVV is encoded by SEQ ID NO: 1.

Embodiment 17. The method of any one of embodiments 1-16, wherein the immune checkpoint inhibitor is administered intravenously.

Embodiment 18. The method of any one of embodiments 1-17, wherein the SVV is administered intratumorally and the immune checkpoint inhibitor is administered intravenously.

Embodiment 19. The method of any one of embodiments 1-18, wherein the immune checkpoint inhibitor is administered every 14 days starting 14 days after initial administration of the SVV.

Embodiment 20. The method of any one of embodiments 1-19, wherein SVV is administered once during a treatment period lasting between about four weeks and 24 weeks.

Embodiment 21. The method of embodiment 20, wherein SVV is administered once during a treatment period lasting about 12 weeks.

Embodiment 22. The method of any one of embodiments 1-19, wherein SVV is administered twice a week, once a week, once every two weeks, once every three weeks, or once every four weeks during a treatment lasting between about four weeks and 24 weeks.

Embodiment 23. The method of embodiment 22, wherein SVV is administered twice a week, once a week, once every two weeks, once every three weeks, or once every four weeks during a treatment lasting about 12 weeks.

Embodiment 24. The method of any one of embodiments 1-23, wherein SVV is administered at a dose between about 2×10³ viral genomes and 5×10¹⁰ viral genomes per subject per day.

Embodiment 25. The method of any one of embodiments 1-24, wherein SVV is administered at a dose between about 2.2×10⁸ viral genomes and 2.2×10¹⁰ viral genomes per subject per day.

Embodiment 26. The method of any one of embodiments 1-25, wherein the immune checkpoint inhibitor is administered intravenously (IV) every 14 days at a treatment dose during the treatment period.

Embodiment 27. The method of any one of embodiments 1-26, wherein the immune checkpoint inhibitor is administered once every four weeks at a maintenance dose following the treatment period.

Embodiment 28. The method of any one of embodiments 1-27, wherein the immune checkpoint inhibitor is nivolumab administered intravenously (IV) every 14 days at a treatment dose during the treatment period.

Embodiment 29. The method of any one of embodiments 1-28, wherein the immune checkpoint inhibitor is nivolumab administered once every four weeks at a maintenance dose following the treatment period.

Embodiment 30. The method of any one of embodiments 1-29, wherein the immune checkpoint inhibitor is nivolumab administered at a treatment dose between about 50 mg and 500 mg during the treatment period.

Embodiment 31. The method of any one of embodiments 1-30, wherein the immune checkpoint inhibitor is nivolumab administered at a treatment dose about 240 mg during the treatment period.

Embodiment 32. The method of any one of embodiments 1-31, wherein the immune checkpoint inhibitor is nivolumab administered at a maintenance dose between about 300 mg and 500 mg following the treatment period.

Embodiment 33. The method of any one of embodiments 1-32, wherein the immune checkpoint inhibitor is nivolumab administered at a maintenance dose about 480 mg following the treatment period.

Embodiment 34. The method of any one of embodiments 1-33, wherein the subject has a neuroendocrine tumor or a neuroendocrine carcinoma selected from large cell neuroendocrine carcinoma, extra thoracic small cell carcinoma, and poorly differentiated neuroendocrine carcinoma.

Embodiment 35. The method of any one of embodiments 1-34, wherein the subject has a neuroendocrine carcinoma with a lesion size having the longest diameter between about 8 mm and 55 mm.

Embodiment 36. The method of any one of embodiments 1-35, wherein the subject has a neuroendocrine carcinoma with a lesion size having the longest diameter between about 10 mm and 50 mm.

Embodiment 37. The method of any one of embodiments 1-36, wherein the subject demonstrates complete response, partial response, or stable disease following the treatment.

Embodiment 38. The method of embodiment 37, wherein complete response comprises disappearance of all known disease determined by two observations not less than four weeks apart.

Embodiment 39. The method of embodiment 37, wherein partial response comprises 30% or more decrease in the total sum of all measurements as determined by two observations not less than four weeks apart.

Embodiment 40. The method of cla embodiment im 37, wherein stable disease comprises a decrease or a less than 20% increase in the total sum of all measurements.

Embodiment 41. The method of any one of embodiments 1-40, wherein total tumor burden in the subject is reduced following treatment.

Embodiment 42. An in vitro method of identifying a cancer that is susceptible to treatment with SVV comprising:
determining the expression of ANTXR1 and one or more biomarkers, or each of the biomarkers, selected from one of the following biomarker profiles in a sample from the cancer:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |
| 15 | ELN, CD248, COL25A1, ALPL, SEMA5B, ATP1B2, NEUROD1, |
| | VAT1L, MFAP4, |

wherein the cancer that is susceptible to the treatment is identified with a sensitivity of at least about 60% and a specificity of at least about 60% and wherein the presence of the one or more biomarkers is indicative of a cancer that may be treated with SVV.

Embodiment 43. The method of embodiment 42, wherein determining the expression uses RNA sequencing (RNAseq), nCounter® platform, or quantitative reverse transcription polymerase chain reaction (qRT-PCR)

Embodiment 44. The in vitro method of embodiment 42 or 43, comprising detecting each of the biomarkers of a biomarker profile.

Embodiment 45. The method of any one of embodiments 42-44, wherein the level of expression of the one or more biomarkers is increased in the sample from the cancer when compared to the level of expression of the one or more biomarkers in a control sample.

Embodiment 46. The method of embodiment 45, wherein the level of expression is increased compared to the level of expression by a cancer that is resistant to treatment with SVV.

Embodiment 47. An in vitro method of identifying a cancer that is susceptible to treatment with SVV comprising detecting the expression of EFS, MFAP4, NTN3, SOX11, NEFH, CNTFR, and JPH4 at at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold greater expression level relative to a housekeeping gene, or relative to a control expression level, and detecting the expression of ANTXR1 in the cancer sample, wherein the cancer that is susceptible to the treatment is identified with a sensitivity of at least about 60% and a specificity of at least about 60%.

Embodiment 48. The method of embodiment 46, wherein the expression of EFS, MFAP4, NTN3, SOX11, NEFH, CNTFR, and JPH4 is increased at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold when compared to the expression level of EFS, MFAP4, NTN3, SOX11, NEFH, CNTFR, and JPH4 in a cancer sample that is resistant to treatment with SVV.

Embodiment 49. The method of any one of embodiments 42 to 48, wherein the cancer sample is a biopsy.

Embodiment 50. The method of embodiment 49, wherein the cancer sample is a formalin-fixed paraffin-embedded (FFPE) tumor block.

Embodiment 51. The method of any one of embodiments 1-50, wherein the SVV is SVV-001, NTX-010, the SVV strain having ATCC Patent Deposit Number PTA-5343, SVV-001/Enhanced IL-2 (SVV-024), SVV-001/Anti-PD-L1 (SVV-012), SVV-001/CXCL9 (SVV-037), SVV-001/TGF beta decoy (SVV-044), SVV-001/Nitroreductase (SVV-058), SVV-001/IL2-IL 15 Fusion Protein (SVV-069), SVV-001/Ovalbumin epitope (SVV-077), or ONCR-788.

Embodiment 52. The method of any one of embodiments 1-51, wherein the cancer is a neuroendocrine cancer, poorly differentiated neuroendocrine carcinoma, well-differentiated neuroendocrine tumor, or a small cell lung cancer (SCLC) tumor.

Embodiment 53. The method of any one of embodiments 1-51, wherein the cancer comprises a neuroendocrine tumor or a neuroendocrine carcinoma selected from large cell neuroendocrine carcinoma, extra thoracic small cell carcinoma, or poorly differentiated neuroendocrine carcinoma.

Embodiment 54. A pharmaceutical composition comprising SVV for use in treating cancer, wherein the cancer expresses ANTXR1 and one or more of the following biomarkers or biomarker profiles:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |
| 15 | ELN, CD248, COL25A1, ALPL, SEMA5B, ATP1B2, NEUROD1, |
| | VAT1L, MFAP4 |

Embodiment 55. The pharmaceutical composition of embodiment 54, wherein the cancer expresses EFS, MFAP4, NTN3, SOX11, NEFH, CNTFR, JPH4, and ANTXR1.

Embodiment 56. Use of pharmaceutical composition for treating cancer, wherein the composition comprises SVV, and wherein the cancer expresses ANTXR1 and one or more of the following biomarkers or biomarker profiles:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |
| 15 | ELN, CD248, COL25A1, ALPL, SEMA5B, ATP1B2, NEUROD1, |
| | VAT1L, MFAP4 |

Embodiment 57. The use of embodiment 56, wherein the cancer expresses EFS, MFAP4, NTN3, SOX11, NEFH, CNTFR, JPH4, and ANTXR1.

Embodiment 58. The use of embodiment 56 or 57, wherein the pharmaceutical composition is used in combination with an immune checkpoint inhibitor.

Embodiment 59. The use of any one of embodiments 56-58, wherein the pharmaceutical composition is used in combination with an immune checkpoint inhibitor nivolumab.

Embodiment 60. The pharmaceutical composition of any one of embodiments 54-55 or the use of any one of embodiments 56-59, wherein the SVV is SVV-001, NTX-010, the SVV strain having ATCC Patent Deposit Number PTA-5343, SVV-001/Enhanced IL-2 (SVV-024), SVV-001/Anti-PD-L1 (SVV-012), SVV-001/CXCL9 (SVV-037), SVV-001/TGF beta decoy (SVV-044), SVV-001/Nitroreductase (SVV-058), SVV-001/IL2-IL15 Fusion Protein (SVV-069), SVV-001/Ovalbumin epitope (SVV-077), or ONCR-788.

Embodiment 61. A kit for use in an in vitro screening method for a cancer that is susceptible for treatment with SVV, wherein the kit comprises one or more reagents for detecting ANTXR1 and one or more biomarkers selected from the following biomarker profiles:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |

Embodiment 62. The kit of embodiment 61, wherein the kit comprises one or more reagents for detecting EFS, MFAP4, NTN3, SOX11, NEFH, CNTFR, JPH4, and ANTXR1.

Embodiment 63. The method of any one of embodiments 1-53, the pharmaceutical composition of any one of embodiments 54, 55, or 60, the use of any one of embodiments 56-60, or the kit of embodiment 61 or 62, wherein the cancer is a neuroendocrine tumor or a neuroendocrine carcinoma selected from large cell neuroendocrine carcinoma, extra thoracic small cell carcinoma, or poorly differentiated neuroendocrine carcinoma.

1.-63. (canceled)

64. A kit for use in an in vitro screening method for a cancer that is susceptible for treatment with SVV, wherein the kit comprises one or more detection reagents for detecting ANTXR1 and one or more biomarkers selected from the following biomarker profiles:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |

65. The kit of claim 64, wherein the one or more detection reagents are suitable for detecting the presence of a polypeptide or a nucleic acid of ANTXR1 and the one or more biomarkers.

66. The kit of claim 64, wherein the one or more detection reagents detect cDNA or mRNA of the ANTXR1 and the one or more biomarkers.

67. The kit of claim 64, wherein the one or more detection reagents comprise a panel of probe sets for detecting the ANTXR1 and the one or more biomarkers.

68. The kit of claim 64, wherein the one or more detection reagents comprise a panel of antibodies for detecting the ANTXR1 and the one or more biomarkers.

69. The kit of claim 64, wherein the detection reagents comprise a panel of probes or a panel of antibodies for detecting EFS, MFAP4, NTN3, SOX11, NEFH, CNTFR, JPH4, and ANTXR1.

70. An in vitro method of identifying a cancer that is susceptible to treatment with SVV comprising:
determining the expression of ANTXR1 and one or more biomarkers, or each of the biomarkers, selected from one of the following biomarker profiles in a sample from the cancer:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |
| 15 | ELN, CD248, COL25A1, ALPL, SEMA5B, ATP1B2, NEUROD1, |
| | VAT1L, MFAP4, |

wherein the cancer that is susceptible to the treatment is identified with a sensitivity of at least about 60% and a specificity of at least about 60% and wherein the presence of the one or more biomarkers is indicative of a cancer that may be treated with SVV.

71. The in vitro method of claim 70, wherein determining the expression uses RNA sequencing (RNAseq), nCounter® platform, or quantitative reverse transcription polymerase chain reaction (qRT-PCR).

72. The in vitro method of claim 70, comprising detecting each of the biomarkers of a biomarker profile.

73. The in vitro method of claim 70, wherein the level of expression of the one or more biomarkers is increased in the sample from the cancer when compared to the level of expression of the one or more biomarkers in a control sample.

74. The in vitro method of claim 73, wherein the level of expression is increased compared to the level of expression by a cancer that is resistant to treatment with SVV.

75. The in vitro method of claim 70 comprising detecting the expression of EFS, MFAP4, NTN3, SOX11, NEFH, CNTFR, and JPH4 at at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold greater expression level relative to a housekeeping gene, or relative to a control expression level, and detecting the expression of ANTXR1 in the cancer sample, wherein the cancer that is susceptible to the treatment is identified with a sensitivity of at least about 60% and a specificity of at least about 60%.

76. The in vitro method of claim 70, wherein the expression of EFS, MFAP4, NTN3, SOX11, NEFH, CNTFR, and JPH4 is increased at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold when compared to the expression level of EFS, MFAP4, NTN3, SOX11, NEFH, CNTFR, and JPH4 in a cancer sample that is resistant to treatment with SVV.

77. The in vitro method of claim 70, wherein the cancer sample is a biopsy or a formalin-fixed paraffin-embedded (FFPE) tumor block.

78. A method of treating a cancer in a human subject in need thereof comprising:
administering to the subject an effective amount of a Seneca Valley Virus (SVV) and an immune checkpoint inhibitor during a treatment period lasting until cancer progression, end of treatment, or unacceptable toxicity,
wherein the subject has cancer expressing one or more biomarkers or each of the biomarkers selected from one of the following biomarker profiles:

| | |
|---|---|
| 1 | ELN, CD248, COL25A1, ALPL, SEMA5B, ATP1B2, NEUROD1, |
| | VAT1L, MFAP4, LPPR4, HPCA, ISLR, NHLH1, CUX2, CAMKV, |
| | ACCN4, JPH4, HIF3A, CNTFR, and NTN3 |
| 2 | ELN, CD248, EFS, ALPL, SEMA5B, ATP1B2, NEUROD1, VAT1L, |
| | MFAP4, NEFH, HPCA, ISLR, NHLH1, SOX11, CAMKV, ACCN4, |
| | JPH4, HIF3A, CNTFR, and NTN3 |
| 3 | ELN, CD248, EFS, ALPL, and SEMA5B |
| 4 | ELN, CD248, EFS, ALPL, SEMA5B, ATP1B2, NEUROD1, VAT1L, |
| | MFAP4, and NEFH |
| 5 | COL25A1, CSF2RA, NCAM1, NDN, ST8SIA2, and TSPYL5 |
| 6 | ATP2B3, COL25A1, HAND2, RUNX1T1, PCDH8, CNTN4, EFS, |
| | SOX11, TSPYL5, GRIK3, NDN, ST8SIA2, and CSAG3 |
| 7 | EFS, MFAP4, NTN3, SOX11, NEFH, CNTFR, and JPH4 |
| 8 | EFS, MFAP4 NTN3, SOX11, NEFH, and CNTFR |
| 9 | ELN, CD248, ALPL, SEMA5B, ATP1B2, NEUROD1, VAT1L, |
| | MFAP4, HPCA, ISLR, NHLH1, CAMKV, and ACCN4 |
| 10 | ELN, CD248, EFS, ALPL, SEMA5B, ATP1B2, NEUROD1, VAT1L, |
| | MFAP4, NEFH, HPCA, ISLR, NHLH1, SOX11, and CAMKV |
| 11 | SOX11, EFS, NEFH, MFAP4, and CNTFR |
| 12 | ELN, CD248, EFS, ALPL, SEMA5B, ATP1B2, NEUROD1, VAT1L, |
| | MFAP4, NEFH, HPCA, ISLR, NHLH1, SOX11 |
| 13 | ELN, CD248, ALPL, SEMA5B, ATP1B2, NEUROD1, VAT1L, |
| | MFAP4, HPCA, ISLR, NHLH1 |
| 14 | SOX11, EFS, NEFH, MFAP4, CNTFR |
| 15 | ELN, CD248, COL25A1, ALPL, SEMA5B, ATP1B2, NEUROD1, |
| | VAT1L, MFAP4, |

and expressing anthrax toxin receptor 1 (ANTXR1).

79. The method of claim 78, wherein the cancer expresses EFS, MFAP4, NTN3, SOX11, NEFH, CNTFR, and JPH4 at at least about two-fold, at least about three-fold, at least about four-fold, at least about five-fold, at least about six-fold, at least about seven-fold, at least about eight-fold, at least about nine-fold, at least about ten-fold, at least about 11-fold, at least about 12-fold, at least about 13-fold, at least about 14-fold, or at least about 15-fold greater expression level relative to a housekeeping gene or relative to a control expression level.

80. The method of claim 78, wherein the cancer expresses the one or more biomarkers at at least about two fold, at least about three fold, at least about four fold, at least about five fold, at least about six fold, at least about seven fold, at least about eight fold, at least about nine fold, at least about ten fold, at least about 11 fold, at least about 12 fold, at least about 13 fold, at least about 14 fold, or at least about 15 fold greater expression level when compared to expression level of the of the one or more biomarkers in cancers that are resistant to treatment with SVV.

81. The method of claim 78, wherein the cancer expresses EFS, MFAP4, NTN3, SOX11, NEFH, CNTFR, JPH4, and ANTXR1 as determined using a gene expression analysis selected from RNA sequencing (RNAseq), nCounter® platform, and quantitative polymerase chain reaction (qPCR).

82. The method of claim 78, wherein the SVV is SVV-001, NTX-010, the SVV strain having ATCC Patent Deposit Number PTA-5343, SVV-001/Enhanced IL-2 (SVV-024), SVV-001/Anti-PD-L1 (SVV-012), SVV-001/CXCL9 (SVV-037), SVV-001/TGF beta decoy (SVV-044), SVV-001/Nitroreductase (SVV-058), SVV-001/IL2-IL15 Fusion Protein (SVV-069), SVV-001/Ovalbumin epitope (SVV-077), or ONCR-788.

83. The method of claim 70, wherein the cancer is a neuroendocrine cancer, poorly differentiated neuroendocrine carcinoma, well-differentiated neuroendocrine tumor, or a small cell lung cancer (SCLC) tumor.
